(19) United States
(12) Patent Application Publication
(10) Pub. No.: US 2023/0073250 A1
(43) Pub. Date: Mar. 9, 2023

(54) Ribozyme-mediated RNA Assembly and Expression

(71) Applicant: University of Rochester, Rochester, NY (US)

(72) Inventor: Douglas Matthew Anderson, Pittsford, NY (US)

(21) Appl. No.: 17/760,213

(22) PCT Filed: Feb. 5, 2021
(86) PCT No.: PCT/US21/16885

§ 371 Date: Aug. 5, 2022

Related U.S. Application Data
(60) Provisional application No. 62/971,356, filed on Feb. 7, 2020.

(51) Int. Cl. C12N 15/113; C12N 9/00; C12N 15/11; C12N 15/66

(52) U.S. Cl. CPC C12N 15/113; C12N 9/93; C12Y 605/01; C12N 15/11; C12N 15/66; C12N 2310/121; C12N 2310/20; A61K 48/00

(57) ABSTRACT

The present invention provides compositions, systems and methods for using ribozyme-mediated cis-cleavage and trans-splicing of RNA molecules to express proteins or fusion proteins of interest.

### CROSS-REFERENCES TO RELATED APPLICATIONS

This application claims priority to U.S. Provisional Patent Application No. 62/971,356 filed on Feb. 7, 2020, the contents of which are incorporated by reference herein in its entirety.

### BACKGROUND OF THE INVENTION

In certain situations, expression of full-length proteins is limited due to the size limitations of plasmids and vectors. For example, in therapeutic settings, some nucleic acids encoding full-length proteins exceed the packaging size for AAV, thereby limiting their applicability in gene therapy settings. Additionally, certain biologically and industrially relevant proteins contain numerous repeats that can make expression difficult.

Thus, there is a need in the art for improved compositions and methods for efficient protein expression. This invention satisfies this unmet need.

### SUMMARY OF THE INVENTION

In one embodiment, the present invention comprises a system for generating an RNA molecule encoding a protein of interest comprising: a nucleic acid molecule encoding a first RNA molecule comprising a coding region encoding a first portion of the protein of interest and a 3′ribozyme; and a nucleic acid molecule encoding a second RNA molecule comprising a coding region encoding a second portion of the protein of interest and a 5′ribozyme.

In one embodiment, the 3′ribozyme catalyzes itself out of the first RNA molecule, thereby generating a 3′P or 2′3′ cP end. In one embodiment, the 5′ribozyme catalyzes itself out of the second RNA molecule, thereby generating a 5′OH end. In one embodiment, the 3′P or 2′3′ cP end is ligated to the 5′OH end to form an RNA molecule comprising the coding region of the first RNA molecule and the coding region of the second RNA molecule. In one embodiment, the 3′ ribozyme is a member of the HDV family of ribozymes. In one embodiment, the 5′ ribozyme is a member of the HH family of ribozymes.

In one embodiment, the system further comprises one or more additional nucleic acid molecules encoding one or more additional RNA molecules, each additional RNA molecule comprising a coding region encoding a domain of the protein of interest; a 5′ ribozyme; and a 3′ ribozyme.

In one embodiment, the system further comprises one or more additional nucleic acid molecules encoding one or more additional RNA molecules, each additional RNA molecule comprising a coding region encoding a domain of the protein of interest; a 5′ ribozyme; and a 3′ ribozyme recognition sequence. In one embodiment, the system further comprises a ribozyme that interacts with the 3′ ribozyme recognition sequence which induces the removal of the 3′ recognition sequence. In one embodiment, the 3′ ribozyme recognition sequence comprises VS-S and wherein the ribozyme is VS-Rz.

In one embodiment, the present invention relates to a method for generating an RNA molecule encoding a protein of interest comprising: administering to a cell or tissue a nucleic acid molecule encoding a first RNA molecule comprising a coding region encoding a first portion of the protein of interest and a 3′ribozyme; and administering to a cell or tissue a nucleic acid molecule encoding a second RNA molecule comprising a coding region encoding a second portion of the protein of interest and a 5′ribozyme.

In one embodiment, the 3′ribozyme catalyzes itself out of the first RNA molecule, thereby generating a 3′P or 2′3′ cP end. In one embodiment, the 5′ribozyme catalyzes itself out of the second RNA molecule, thereby generating a 5′OH end. In one embodiment, the 3′P or 2′3′ cP end is ligated to the 5′OH end to form an RNA molecule comprising the coding region of the first RNA molecule and the coding region of the second RNA molecule. In one embodiment, the 3′ ribozyme is a member of the HDV family of ribozymes. In one embodiment, the 5′ ribozyme is a member of the HH family of ribozymes.

In one embodiment, the method further comprises administering to the cell or tissue one or more additional nucleic acid molecules encoding one or more additional RNA molecules, each additional RNA molecule comprising a coding region encoding a domain of the protein of interest; a 5′ ribozyme; and a 3′ ribozyme.

In one embodiment, the method further comprises administering to the cell or tissue one or more additional nucleic acid molecules encoding one or more additional RNA molecules, each additional RNA molecule comprising a coding region encoding a domain of the protein of interest; a 5′ ribozyme; and a 3′ ribozyme recognition sequence. In one embodiment, the method further comprises administering to the cell or tissue a ribozyme that interacts with the 3′ ribozyme recognition sequence which induces the removal of the 3′ recognition sequence. In one embodiment, the 3′ ribozyme recognition sequence comprises VS-S and wherein the ribozyme is VS-Rz. In one embodiment, the method further comprises administering to the cell or tissue a ligase to induce the assembly of the RNA molecule. In one embodiment, the ligase is RNA 2′,3′-Cyclic Phosphate and 5′-OH (RtcB) ligase.

In one embodiment, the present invention comprises an in vitro method of generating an RNA molecule encoding a protein of interest comprising: providing a first RNA molecule comprising a coding region encoding a first portion of the protein of interest and a 3′ribozyme; providing a second RNA molecule comprising a coding region encoding a second portion of the protein of interest and a 5′ribozyme; and providing a ligase to induce the assembly of the RNA molecule from the coding region of the first RNA molecule and the coding region of the second RNA molecule.

In one embodiment, the present invention comprises an in vitro method of generating an RNA molecule encoding a repeat domain protein of interest comprising the steps of: a) providing a first RNA molecule comprising a coding region encoding a first portion of the protein of interest and a 3′ribozyme; b) providing one or more additional RNA molecule comprising a coding region encoding a domain of the protein of interest, a 5′ ribozyme, and a 3′ ribozyme recognition sequence; c) providing a ligase to ligate the coding region of the first RNA molecule and the coding region of the one or more additional RNA molecule; d) providing a ribozyme that recognizes the 3′ ribozyme recognition sequence and catalyzes its removal; e) repeating steps b)-d) one or more times to generate an RNA molecule encoding a plurality of repeat domains; f) providing a last RNA molecule comprising a coding region encoding a last portion of the protein of interest and a 5′ribozyme; and g) providing a ligase to ligate the coding region of the one or more additional RNA molecule and the coding region of the last RNA molecule, thereby generating a complete RNA molecule encoding a repeat domain protein.

In one embodiment, the present invention comprises a method of treating a disease or disorder in a subject caused by a mutation in a large protein of interest comprising: administering to said subject a first nucleic acid molecule comprising a coding region encoding a first portion of the protein of interest and a 3′ribozyme; and administering to said subject a second nucleic acid comprising a coding region encoding a second portion of the protein of interest and a 5′ribozyme.

In one embodiment, the disease or disorder is one or more selected from the group consisting of: Duchenne Muscular Dystrophy, autosomal recessive polycystic kidney disease, Hemophilia A, Stargardt macular degeneration, limb-girdle muscular dystrophies , DFNB9, neurosensory nonsyndromic recessive deafness, Cystic Fibrosis, Wilson Disease, Miyoshi Muscular Dystrophy and Deafness, Autosomal Recessive 9, Usher Syndrome, Type I and Deafness, Autosomal Recessive 2, Deafness, Autosomal Recessive 3 and Nonsyndromic Hearing Loss, Usher syndrome type I, autosomal recessive deafness-16 (DFNB16), Meniere's disease (MD), Deafness, Autosomal Dominant 12 and Deafness, Autosomal Recessive 21, Usher syndrome Type 1F (USH1F) and DFNB23, Deafness, Autosomal Recessive 28 and Nonsyndromic Hearing Loss, Deafness, Autosomal Recessive 30 and Nonsyndromic Hearing Loss, Otospondylomegaepiphyseal Dysplasia, Autosomal Recessive and Otospondylomegaepiphyseal Dysplasia, Autosomal Dominant, Deafness, Autosomal Recessive 77 and Autosomal Recessive Non-Syndromic Sensorineural Deafness Type Dfnb, autosomal-recessive nonsyndromic hearing impairment DFNB84, Deafness, Autosomal Recessive 84B and Rare Genetic Deafness, Peripheral Neuropathy, Myopathy, Hoarseness, And Hearing Loss and Deafness, Autosomal Dominant 4A, congenital thrombocytopenia, sensory hearing loss, DFNA56, HXB, deafness, autosomal dominant 56, hexabrachion , epileptic encephalopathy, Timothy Syndrome and Long Qt Syndrome8, X-linked retinal disorder, Hyperaldosteronism, Spinocerebellar Ataxia 42, Primary Aldosteronism, Seizures, And Neurologic Abnormalities and Sinoatrial Node Dysfunction And Deafness, Neurodevelopmental Disorder, hypokalemic periodic paralysis, Epilepsy, developmental and epileptic encephalopathies, Brody myopathy, Darier's disease/ Heart disease, von Willebrand disease, and Zellweger syndrome.

In one embodiment, the present invention comprises a system for generating an RNA molecule encoding a protein of interest and a circular RNA molecule comprising a nucleic acid encoding: a first portion of a protein of interest; a synthetic intron comprising a 5′ ribozyme, a cargo sequence, and a 3′ ribozyme; and a second portion of a protein of interest.

In one embodiment, the protein of interest is one or more selected from the group consisting of: a therapeutic protein, a reporter protein, and a Cas9 protein.

In one embodiment, the cargo sequence is one or more selected from the group consisting of: a sequence encoding a therapeutic protein of interest, a CRISPR guide RNA sequence, a small RNA sequence, and a trans-cleaving ribozyme sequence. In one embodiment, said small RNA sequence comprises one or more selected from the group consisting of: microRNA (miRNA), Piwi-interacting RNA (piRNA), small interfering RNA (siRNA), small nucleolar RNA (snoRNAs), small tRNA-derived RNA (tsRNA), small rDNA-derived RNA (srRNA) and small nuclear RNA (snRNA).

In one embodiment, the 3′ ribozyme of the synthetic intron is a member of the HH family of ribozymes. In one embodiment, the 5′ ribozyme of the synthetic intron is one or more selected from the group consisting of: a member of the HDV family of ribozymes, a member of the HDV family of ribozymes, and VS-S ribozyme recognition sequence. In one embodiment, the sytem further comprises one or more selected from the group consisting of: RtcB ligase and a nucleic acid encoding RtcB ligase.

In one embodiment, the present invention comprises a method of delivering an RNA molecule encoding a protein of interest and a circular RNA molecule, the method comprising: administering to a cell or tissue a nucleic acid encoding a first portion of a protein of interest, a synthetic intron comprising a cis-cleaving 5′ ribozyme, a cargo sequence and a cis-cleaving 3′ ribozyme, and a second portion of a protein of interest.

In one embodiment, the protein of interest is one or more selected from the group consisting of: a therapeutic protein, a reporter protein, and a Cas9 protein.

In one embodiment, the cargo sequence is one or more selected from the group consisting of: a sequence encoding a therapeutic protein of interest, a CRISPR guide RNA sequence, a small RNA sequence, and a trans-cleaving ribozyme sequence. In one embodiment, said small RNA sequence comprises one or more selected from the group consisting of: microRNA (miRNA), Piwi-interacting RNA (piRNA), small interfering RNA (siRNA), small nucleolar RNA (snoRNAs), small tRNA-derived RNA (tsRNA), small rDNA-derived RNA (srRNA) and small nuclear RNA (snRNA).

In one embodiment, the method further comprises administering to the cell or tissue one or more selected from the group consisting of: RtcB ligase and a nucleic acid encoding RtcB ligase.

### BRIEF DESCRIPTION OF THE DRAWINGS

The following detailed description of embodiments of the invention will be better understood when read in conjunction with the appended drawings. It should be understood that the invention is not limited to the precise arrangements and instrumentalities of the embodiments shown in the drawings.

FIG. 1, comprising FIG. 1A through FIG. 1E, depicts ribozyme-mediated trans-splicing and expression in mammalian cells. FIG. 1A shows a diagram depicting the vectors encoding the N-terminal (Nt) half of GFP with 3′ HDV ribozyme and C-terminal (Ct) half of GFP with 5′ Hammerhead (HH) ribozyme. FIG. 1B depicts exemplary results demonstrating that co-expression of both Nt-GFP-HDV and HH-Ct-GFP in COS7 and HEK293T cells resulted in detectable GFP fluorescence, but not when transfected separately. FIGS. 1C-1D depict exemplary results of RT-PCR amplification (FIG. 1C) and sanger sequence analysis (FIG. 1D) using primers specific to each independent RNA (G1 and G2), showing removal of the ribozymes and scar-less trans-splicing and restoration of the GFP coding sequence. FIG. 1E depicts exemplary Western blot results using an antibody specific to GFP showing the full-length protein size predicted for GFP.

FIG. 2, comprising FIGS. 2A through FIG. 2E, depicts the development of a luciferase-based reporter to quantify the impact of ribozyme sequences on trans-splicing in mammalian cells. FIG. 2A shows a diagram depicting the vectors encoding the N-terminal (Nt) half of Luciferase with 3′ HDV ribozyme and C-terminal (Ct) half of Luciferase with 5′ Hammerhead (HH) ribozyme. FIGS. 2B-2C depict exemplary results of RT-PCR amplification (FIG. 2B) and sanger sequence analysis (FIG. 2C) using primers specific to each independent Luc RNA (L1 and L2), showing removal of the ribozymes and scar-less trans-splicing of the luciferase open reading frame. FIGS. 2D-2E demonstrate the impact of different HDV (FIG. 2D) and HH (FIG. 2E) ribozyme sequences on trans-splicing in mammalian cells. In addition, mutation of ribozyme catalytic nucleotides resulted in loss of luciferase activity (FIG. 2D, last column, and FIG. 2E, last column).

FIG. 3, comprising FIG. 3A through FIG. 3D, demonstrates the regulation of protein expression from Nt and Ct vectors. FIG. 3A shows a diagram depicting placement of C-terminal protein degradation sequences which prevent expression of Nt vector encoded proteins. FIG. 3B depicts exemplary results demonstrating the efficiency of different protein degradation sequences at preventing GFP-HDV expression from Nt vector encoding full length GFP. FIG. 3C shows a diagram depicting placement of N-terminal translational control sequences to prevent translation of protein sequences in Ct vectors. FIG. 3D depicts exemplary results demonstrating the efficiency of different GFP sequence modifications or translational control sequences at preventing GFP fluorescence in mammalian cells.

FIG. 4, comprising FIG. 4A through FIG. 4D, demonstrates single and multiplex ribozyme-mediated trans-splicing in mammalian cells. FIG. 4A shows a diagram depicting vectors encoding a 4×MTS and full length GFP (no start ATG codon) with ribozymes to mediate trans-splicing and expression of a mitochondrial targeted GFP protein. FIG. 4B depicts exemplary results demonstrating that co-expression of these vectors results in mitochondrial localized green fluorescence which overlapped with the red fluorescence of mitotracker CMXRos. FIG. 4C shows a diagram depicting vectors for multiplex tran-splicing and expression of a mitochondrial targeted GFP protein (4×MTS-GFP) in reading frame 1 and a myristoylation membrane targeted red fluorescent protein (F2-Myr-RFP) in reading frame 2. FIG. 4D depicts exemplary results demonstrating that co-expression of all four vectors in mammalian Cos? cells results in specific green fluorescence in mitochondrial and red fluorescence in membranes.

FIG. 5, comprising FIG. 5A and FIG. 5B, demonstrates enhanced ribozyme-mediated trans splicing using optimized ribozyme sequences and cis-splicing splice acceptor and splice donor sequences. FIG. 5A shows a diagram depicting the placement of chimeric splice donor (SD) and splice acceptor (SA) sequences in a generic Nt-GFP-3′Rz and 5′ Rz-Ct-GFP trans-splicing GFP reporter, wherein Rz denotes an cis-cleaving ribozyme. FIG. 5B depicts exemplary results of GFP fluorescence in Cos7 cells after single vector transfection (first two columns) or co-transfection (last two columns) 18 hours post-transfection (first three columns) or 36 hours (last column) post-transfection. The first row depicts the use of unoptimized HH and HDV ribozymes, second row depicts the use of optimized Twister and RzB ribozymes, and the last row depicts to the combination of Twister and RzB ribozymes and SD and SA sequences.

FIG. 6 comprising FIG. 6A through FIG. 6D, demonstrates ribozyme-mediated trans splicing of large protein coding genes. FIG. 6A shows a diagram depicting vectors encoding a split μDystrophin-GFP fusion protein for delivery using AAV vector. FIGS. 6B-6C depicts exemplary results of RT-PCR (FIG. 6B) and sanger sequencing (FIG. 6C) analyses on cells transfected with Nt-Dys and Ct-Dys vectors showing specific trans-splicing. FIG. 6D depicts exemplary results of GFP fluorescence from cells transfected with both Nt and Ct Dystrophin vectors imaged using confocal microscopy showing the predicted membrane localization of Dystrophin.

FIG. 7, comprising FIG. 7A through FIG. 7C, demonstrates lentiviral delivery of ribozyme-containing RNAs for trans-splicing in target cells. FIG. 7A shows a diagram depicting the negative sense orientation of Nt and Ct split GFP expression cassette in the lentiviral gene transfer vector. FIG. 7B depicts exemplary results demonstrating that only cells co-transduced with lentivirus encoding both Nt-GFP and Ct-GFP genes show GFP fluorescence. FIG. 7C shows a diagram depicting the negative sense orientation of Nt and Ct split Dys expression cassette in the lentiviral gene transfer vector.

FIG. 8, comprising FIG. 8A and FIG. 8B, demonstrates ribozyme-mediated trans-splicing and expression of the toxic DTA gene. FIG. 8A shows a diagram depicting vectors encoding a split Nt and Ct DTA gene. FIG. 8B depicts exemplary results demonstrating that cells co-transfected with both Nt-DTA and Ct-DTA result in decreased expression of a co-transfected GFP reporter, consistent with the translational repressor function of DTA in mammalian cells.

FIG. 9 depicts exemplary results demonstrating that co-expression of exogenous RNA modulating enzymes can enhance or inhibit ribozyme-mediated trans-splicing in mammalian cells.

FIG. 10, comprising FIG. 10A through FIG. 10D, demonstrates that RtcB is sufficient to catalyze ribozyme-mediated trans-splicing in vitro. FIG. 10A shows a diagram depicting a split luciferase trans-splicing reporter which contains an upstream T7 RNA promoter to allow for in vitro RNA transcription. FIG. 10B shows exemplary RT-PCR results demonstrating that in vitro trans-spliced luciferase RNA is dependent upon addition of RtcB protein (NEB) using the manufacturer's recommended reaction conditions. FIG. 10C shows a diagram depicting a trans-splicing vector for conserved N-terminal (N1L) and C-terminal (N3R) domains of Spidroin. FIG. 10D depicts exemplary sanger sequencing results demonstrating that RtcB ligase from E. coli was sufficient to catalyze the trans-ligation of the ribozyme cleaved N1L and N3R encoding RNAs.

FIG. 11 depicts the in vitro directional ligation of ribozyme-catalyzed RNAs using RtcB, VS-S and VS-Rz.

FIG. 12, comprising FIG. 12A through 12D, depicts the use of trans-cleaving ribozymes for trans-splicing of RNA. FIG. 12A depicts secondary structures of ribozymes which cleave in cis. FIG. 12B depicts engineered ribozymes capable of cleaving in trans. FIG. 12C and FIG. 12D depict diagrams demonstrating potential applications of trans-cleaving ribozymes to delete disease causing mutations, such as frame-shifting or premature stop codons, to restore protein expression and function.

FIG. 13, comprising FIG. 13A and FIG. 13B, depicts the secondary structures of representative ribozymes which can be utilized for scar-less trans-splicing of RNA. FIG. 13A depicts representative ribozymes which can be used for scar-less 5′ cleavage. FIG. 13B depicts representative ribozymes which can be used for scar-less 3′ cleavage. N=any nucleotide. Red scissors demarcate a cleavage site. Red nucleotides indicate catalytic mutations. Orange nucleotides represent RNA sequence to be trans-spliced. Dark blue nucleotides indicate ribozyme sequence required to form stem. Light blue indicates tertiary stabilizing motif (TSM) in stem 1 which interacts with stem 2 loop. HH—Hammerhead, HDV—Hepatitis Delta Virus, Rz—ribozyme.

FIG. 14, comprising FIG. 14A through FIG. 14C, depicts scar-less cleavage and inducible RNA trans-splicing and expression with trans-activating ribozymes. FIG. 14A depicts a diagram showing that the VS ribozyme can be split into two components, a small VS-S stem loop, which lacks autocatalytic activity, and larger VS-Rz, which induces VS-S cleavage when delivered in trans. The VS-S/VS-Rz ribozyme pair can be utilized to generate inducible scar-less trans-splicing. FIG. 14B shows a diagram depicting a method to utilize the VS-S/VS-Rz trans-activated ribozyme pair to generate an inducible RNA trans-splicing system. Only upon delivery or expression of VS-Rz, does the Nt-GFP-VS-S RNA generate a suitable RNA terminus that can participate in trans-splicing with the co-expressed Ct-GFP RNA. FIG. 14C shows a diagram depicting a method to generate an RNA with an N-terminal sequence, a variable or non-variable repeat region, and C-terminal sequence. The ‘repeat’ RNA contains a 5′ autocatalytic ribozyme and a 3′ trans-activated ribozyme, such as VS-S, which allows for controlled repeat addition dependent upon the selective addition of trans-activating VS-Rz and ligase, such as RtcB.

FIG. 15, comprising FIG. 15A through FIG. 15E, depicts ribozyme-mediated trans-splicing with generation of stable intronic RNA sequences. FIG. 15A shows a diagram depicting the use of cis-cleaving ribozymes to mediate the trans-splicing of two independent RNAs. FIG. 15B shows a diagram depicting the use of internal cis-cleaving ribozymes to create a synthetic intron. FIG. 15C depicts exemplary results demonstrating efficient cis-cleavage of a synthetic intron and trans-splicing of independent RNAs to yield functional protein (GFP). FIG. 15D and FIG. 15E show diagrams depicting the use of internal cis-cleaving ribozymes to generate a trans-spliced and translated reporter and intronic sequence, ‘cargo’, which could be any useful RNA sequence or gene expression cassette.

FIG. 16, comprising FIG. 16A through FIG. 16C, depicts exemplary results of optimized ribozyme sequences for ribozyme-mediated trans-splicing in vivo. FIG. 16A depicts a comparison of the relative ribozyme activity using a Luciferase trans-splicing reporter. The RzB Hammerhead ribozyme variant, containing a tertiary stabilizing motif and active in low magnesium concentrations, showed the greatest luciferase activity in mammalian cells. FIG. 16B depicts a comparison of HDV ribozymes (HDV68 and Genomic HDV with a Twister ribozyme (Twst). A Twister ribozyme on the 3′ end of Nt-Luc provided the greatest luciferase activity, which was abolished with catalytic inactivating mutations (Twst mut). FIG. 16C depicts a comparison of Twister ribozyme sequence modifications. Shortening of the P1 stem decreased reporter activity. Modification of the first residue revealed that Twister can tolerate an A nucleotide at position 1 (U1A).

### DETAILED DESCRIPTION

### Definitions

Unless defined otherwise, all technical and scientific terms used herein have the same meaning as commonly understood by one of ordinary skill in the art to which this invention belongs.

Generally, the nomenclature used herein and the laboratory procedures in cell culture, molecular genetics, organic chemistry, and nucleic acid chemistry and hybridization are those well-known and commonly employed in the art.

Standard techniques are used for nucleic acid and peptide synthesis. The techniques and procedures are generally performed according to conventional methods in the art and various general references (e.g., Sambrook and Russell, 2012, Molecular Cloning, A Laboratory Approach, Cold Spring Harbor Press, Cold Spring Harbor, N.Y., and Ausubel et al., 2012, Current Protocols in Molecular Biology, John Wiley & Sons, NY), which are provided throughout this document.

The nomenclature used herein and the laboratory procedures used in analytical chemistry and organic syntheses described below are those well-known and commonly employed in the art. Standard techniques or modifications thereof are used for chemical syntheses and chemical analyses.

The term “a,” “an,” “the” and similar terms used in the context of the present invention (especially in the context of the claims) are to be construed to cover both the singular and plural unless otherwise indicated herein or clearly contradicted by the context.

“About” as used herein when referring to a measurable value such as an amount, a temporal duration, and the like, is meant to encompass variations of ±20%, or ±10%, or ±5%, or ±1%, or ±0.1% from the specified value, as such variations are appropriate to perform the disclosed methods.

“Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences.

When referring to immobilization of molecules (e.g. nucleic acid molecules) to a solid support, the term “attached” as used herein is intended to encompass direct or indirect, covalent or non-covalent attachment, unless indicated otherwise, either explicitly or by context.

As used herein interchangeably, “microspheres”, “beads” or grammatical equivalents thereof describe small discrete particles capable of acting a solid support for attachment of a biomolecule (e.g., a nucleic acid molecule).

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a sign or symptom of the disease or disorder, the frequency with which such a sign or symptom is experienced by a patient, or both, is reduced.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The terms “patient,” “subject,” “individual,” and the like are used interchangeably herein, and refer to any animal or cell whether in vitro or in vivo, amenable to the methods described herein. In one embodiment, the subjects include vertebrates and invertebrates. Invertebrates include, but are not limited to, Drosophila melanogaster and Caenorhabditis elegans. Vertebrates include, but are not limited to, primates, rodents, domestic animals or game animals. Primates include, but are not limited to, chimpanzees, cynomologous monkeys, spider monkeys, and macaques (e.g., Rhesus). Rodents include, but are not limited to, mice, rats, woodchucks, ferrets, rabbits and hamsters. Domestic and game animals include, but are not limited to, cows, horses, pigs, deer, bison, buffalo, feline species (e.g., domestic cat), canine species (e.g., dog, fox, wolf), avian species (e.g., chicken, emu, ostrich), and fish (e.g., zebrafish, trout, catfish and salmon). In some embodiments, the subject is a mammal, e.g., a primate, e.g., a human. In certain non-limiting embodiments, the patient, subject or individual is a human.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific.

In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

A “coding region” of a mRNA molecule also consists of the nucleotide residues of the mRNA molecule which are matched with an anti-codon region of a transfer RNA molecule during translation of the mRNA molecule or which encode a stop codon. The coding region may thus include nucleotide residues comprising codons for amino acid residues which are not present in the mature protein encoded by the mRNA molecule (e.g., amino acid residues in a protein export signal sequence).

“Complementary” as used herein to refer to a nucleic acid, refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. In one embodiment, the first region comprises a first portion and the second region comprises a second portion, whereby, when the first and second portions are arranged in an antiparallel fashion, at least about 50%, at least about 75%, at least about 90%, or at least about 95% of the nucleotide residues of the first portion are capable of base pairing with nucleotide residues in the second portion. In one embodiment, all nucleotide residues of the first portion are capable of base pairing with nucleotide residues in the second portion.

The term “DNA” as used herein is defined as deoxyribonucleic acid.

The term “expression” as used herein is defined as the transcription and/or translation of a particular nucleotide sequence driven by its promoter.

The term “expression vector” as used herein refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules, siRNA, ribozymes, and the like. Expression vectors can contain a variety of control sequences, which refer to nucleic acid sequences necessary for the transcription and possibly translation of an operatively linked coding sequence in a particular host organism. In addition to control sequences that govern transcription and translation, vectors and expression vectors may contain nucleic acid sequences that serve other functions as well.

As used herein the term “wild type” is a term of the art understood by skilled persons and means the typical form of an organism, strain, gene or characteristic as it occurs in nature as distinguished from mutant or variant forms.

The term “homology” refers to a degree of complementarity. There may be partial homology or complete homology (i.e., identity). Homology is often measured using sequence analysis software (e.g., Sequence Analysis Software Package of the Genetics Computer Group. University of Wisconsin Biotechnology Center. 1710 University Avenue. Madison, Wis. 53705). Such software matches similar sequences by assigning degrees of homology to various substitutions, deletions, insertions, and other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

“Isolated” means altered or removed from the natural state. For example, a nucleic acid or a peptide naturally present in its normal context in a living animal is not “isolated,” but the same nucleic acid or peptide partially or completely separated from the coexisting materials of its natural context is “isolated.” An isolated nucleic acid or protein can exist in substantially purified form, or can exist in a non-native environment such as, for example, a host cell.

The term “isolated” when used in relation to a nucleic acid, as in “isolated oligonucleotide” or “isolated polynucleotide” refers to a nucleic acid sequence that is identified and separated from at least one contaminant with which it is ordinarily associated in its source. Thus, an isolated nucleic acid is present in a form or setting that is different from that in which it is found in nature. In contrast, non-isolated nucleic acids (e.g., DNA and RNA) are found in the state they exist in nature. For example, a given DNA sequence (e.g., a gene) is found on the host cell chromosome in proximity to neighboring genes; RNA sequences (e.g., a specific mRNA sequence encoding a specific protein), are found in the cell as a mixture with numerous other mRNAs that encode a multitude of proteins. However, isolated nucleic acid includes, by way of example, such nucleic acid in cells ordinarily expressing that nucleic acid where the nucleic acid is in a chromosomal location different from that of natural cells, or is otherwise flanked by a different nucleic acid sequence than that found in nature. The isolated nucleic acid or oligonucleotide may be present in single-stranded or double-stranded form. When an isolated nucleic acid or oligonucleotide is to be utilized to express a protein, the oligonucleotide contains at a minimum, the sense or coding strand (i.e., the oligonucleotide may be single-stranded), but may contain both the sense and anti-sense strands (i.e., the oligonucleotide may be double-stranded).

The term “isolated” when used in relation to a polypeptide, as in “isolated protein” or “isolated polypeptide” refers to a polypeptide that is identified and separated from at least one contaminant with which it is ordinarily associated in its source. Thus, an isolated polypeptide is present in a form or setting that is different from that in which it is found in nature. In contrast, non-isolated polypeptides (e.g., proteins and enzymes) are found in the state they exist in nature.

By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil). The term “nucleic acid” typically refers to large polynucleotides.

Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

By “expression cassette” is meant a nucleic acid molecule comprising a coding sequence operably linked to promoter/regulatory sequences necessary for transcription and, optionally, translation of the coding sequence.

The term “operably linked” as used herein refer to the linkage of nucleic acid sequences in such a manner that a nucleic acid molecule capable of directing the transcription of a given gene and/or the synthesis of a desired protein molecule is produced. The term also refers to the linkage of sequences encoding amino acids in such a manner that a functional (e.g., enzymatically active, capable of binding to a binding partner, capable of inhibiting, etc.) protein or polypeptide is produced.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a n inducible manner.

As used herein, “stringent conditions” for hybridization refer to conditions under which a nucleic acid having complementarity to a target sequence predominantly hybridizes with the target sequence, and substantially does not hybridize to non-target sequences. Stringent conditions are generally sequence-dependent, and vary depending on a number of factors. In general, the longer the sequence, the higher the temperature at which the sequence specifically hybridizes to its target sequence. Non-limiting examples of stringent conditions are described in detail in Tijssen (1993), Laboratory Techniques In Biochemistry And Molecular Biology-Hybridization With Nucleic Acid Probes Part 1, Second Chapter “Overview of principles of hybridization and the strategy of nucleic acid probe assay”, Elsevier, N.Y.

“Hybridization” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. The complex may comprise two strands forming a duplex structure, three or more strands forming a multi stranded complex, a single self-hybridizing strand, or any combination of these. A hybridization reaction may constitute a step in a more extensive process, such as the initiation of PCR, or the cleavage of a polynucleotide by an enzyme. A sequence capable of hybridizing with a given sequence is referred to as the “complement” of the given sequence.

An “inducible” promoter is a nucleotide sequence which, when operably linked with a polynucleotide which encodes or specifies a gene product, causes the gene product to be produced substantially only when an inducer which corresponds to the promoter is present.

A “constitutive” promoter is a nucleotide sequence which, when operably linked with a polynucleotide which encodes or specifies a gene product, causes the gene product to be produced in a cell under most or all physiological conditions of the cell.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “RNA” as used herein is defined as ribonucleic acid.

The term “ribozyme”, as used herein, refers to an RNA molecule capable of acting as an enzyme. For example, some ribozymes are capable of cleaving RNA molecules. RNA cleaving ribozymes typically consist at least of a catalytic domain and a recognition sequence that is recognized by the catalytic domain. The catalytic domain can be a part of the same RNA molecule as the recognition sequence, and thus mediate cis-cleavage. Alternatively, the catalytic domain can be a separate RNA molecule from the RNA molecule comprising the recognition sequence, and thus mediate trans-cleavage.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide may serve a non-coding function (e.g., promoter, origin of replication, ribosome-binding site, etc.) as well.

The term “recombinant polypeptide” as used herein is defined as a polypeptide produced by using recombinant DNA methods.

As used herein, the terms “solid surface,” “solid support” and other grammatical equivalents thereof refer to any material that is appropriate for or can be modified to be appropriate for the attachment of a biomolecule (e.g., a nucleic acid molecule).

As used herein, the term “tag” refers to any chemical modification of a biomolecule (e.g., a nucleic acid molecule) that provides additional functionality (e.g., attachment to a solid support, fluorescence visualization, etc.).

“Variant” as the term is used herein, is a nucleic acid sequence or a peptide sequence that differs in sequence from a reference nucleic acid sequence or peptide sequence respectively, but retains essential biological properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide can differ in amino acid sequence by one or more substitutions, additions, deletions in any combination. A variant of a nucleic acid or peptide can be a naturally occurring such as an allelic variant, or can be a variant that is not known to occur naturally. Non-naturally occurring variants of nucleic acids and peptides may be made by mutagenesis techniques or by direct synthesis.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The present invention provides compositions and methods for efficiently and reliably ligating two or more individual RNA molecules to produce a larger single RNA molecule that encodes proteins and fusion proteins. The invention utilizes ribozyme-mediated trans-splicing of multiple RNA molecules to assemble a single RNA molecule encoding a protein or fusion protein of interest. The present invention can be used to efficiently produce fusion proteins, chimeric proteins, and the like. Further, the present invention is useful in producing large full-length proteins whose coding sequence may be too large to package into a single vector. Further, the technology of the present invention also allows for the rapid and easy combination of two different sequences, which could have a multiplier effect for generating novel protein combinations or library sequences. This may be particularly useful, for example, for generating synthetic antibodies (like nanobodies) or for functional selection of enzymes.

The present invention also provides compositions and methods for efficiently delivering one or more RNA molecule with a ribozyme-flanked synthetic intron. The ribozyme-flanked synthetic intron can be placed between a first RNA portion encoding an N-terminal portion of a protein of interest and a second RNA portion encoding a C-terminal portion of a protein of interest. The ribozyme-flanked synthetic intron can comprise a cargo sequence, for example, a sequence encoding a therapeutic protein or comprising a functional RNA. The use of two ribozymes allows cis-splicing to generate three RNA fragments: 1) the first RNA portion encoding an N-terminal portion of a protein of interest, 2) the ribozyme-flanked synthetic intron, and 3) second RNA portion encoding a C-terminal portion of a protein of interest. Said cis-splicing generates compatible ends for ligation. Ligation of the compatible ends of the cis-spliced synthetic intron generates a circular RNA molecule, more resistant to degradation than a linear RNA molecule. Ligation of the compatible ends of the first RNA portion encoding an N-terminal portion of a protein of interest and the second RNA portion encoding a C-terminal portion of a protein of interest, generates an RNA molecule encoding a full-length protein of interest. The full-length protein of interest can be, for example, a therapeutic protein, CRISPR-Cas protein, or reporter protein to provide a proxy indicator for delivery and expression of the cargo sequence in the circular RNA molecule comprising the ribozyme-flanked synthetic intron.

In one aspect, the present invention provides one or more nucleic acid molecules encoding two or more RNA molecules. In certain embodiments, one or more of the RNA molecules comprise a ribozyme. In one embodiment, one or more of the RNA molecules comprise a coding region and a ribozyme. In certain embodiments, the ribozyme self-cleaves out of the RNA molecule leaving the coding region. Exemplary ribozymes that may be used in the context of the present invention include, but is not limited to, members of the Hammerhead (HH), Hepatitis Delta Virus (HDV), Varkud Satellite (VS), Sister, Twister-sister, Hairpin, Hatchet and Pistol families of ribozymes.

For example, in one embodiment, the composition comprises a nucleic acid molecule encoding a first RNA molecule, where the first RNA molecule comprises a coding region and a 3′ ribozyme, where the 3′ ribozyme is able to catalyze itself out of the RNA molecule leaving the coding region with a 3′P or 2′3′ cyclic phosphate (cP) end. In one embodiment, the 3′ ribozyme comprises an HDV ribozyme. Further, in one embodiment, the composition comprises a nucleic acid molecule encoding a second RNA molecule, where the second RNA molecule comprises a coding region and a 5′ ribozyme, where the 5′ ribozyme is able to catalyze itself out of the RNA molecule leaving the coding region with a 5′OH end. In one embodiment, the 5′ ribozyme comprises an HH ribozyme. In certain instances, a ligase joins the coding region of the first RNA molecule to the coding region of the second RNA molecule together to form a longer RNA molecule encoding a protein of interest.

For example, in one embodiment, the composition comprises a first RNA molecule, where the first RNA molecule comprises a coding region and a 3′ ribozyme, where the 3′ ribozyme is able to catalyze itself out of the RNA molecule leaving the coding region with a 3′P or 2′3′ cyclic phosphate (cP) end. In one embodiment, the 3′ ribozyme comprises an HDV ribozyme. Further, in one embodiment, the composition comprises a second RNA molecule, where the second RNA molecule comprises a coding region and a 5′ ribozyme, where the 5′ ribozyme is able to catalyze itself out of the RNA molecule leaving the coding region with a 5′OH end. In one embodiment, the 5′ ribozyme comprises an HE ribozyme. In certain instances, a ligase joins the coding region of the first RNA molecule to the coding region of the second RNA molecule together to form a longer RNA molecule encoding a protein of interest.

In certain embodiments the first RNA comprises a coding region encoding a first portion of the protein of interest and the second RNA comprises a coding region encoding a second portion of the protein of interest, and thus the ribozyme-mediated cleavage and ligase-mediated assembly of the RNA molecules results in the production of an RNA molecule encoding a protein having both the first and second portions. The present invention can be used to produce full-length proteins from multiple RNAs, each comprising a coding region encoding a portion of the full-length protein. Further, the present invention can be used to produce fusion proteins comprising multiple domains, where each RNA molecule comprises a coding region encoding a domain of the fusion protein. For example, the present invention can be used to generate an RNA molecule encoding a protein having a leader sequence, N-terminal tag, C-terminal tag, or the like by assembling an RNA from a first RNA comprising a coding sequence encoding the leader sequence, N-terminal tag, or C-terminal tag, and a second RNA molecule comprising a coding sequence encoding the protein.

In certain embodiments, the invention relates to formation of a single RNA molecule from three or more individual RNA molecules. For example, in certain aspects, the composition comprise a nucleic acid molecule encoding a first RNA molecule, where the first RNA molecule comprises a coding region encoding the N-terminal region of a protein; a nucleic acid molecule encoding a second RNA molecule, where the second RNA molecule comprises a coding region encoding the C-terminal region of a protein; and one or more nucleic acid molecules encoding one or more additional RNA molecules, each comprising a coding region encoding a protein domain (e.g., repeat domain). In one embodiment, the first RNA molecule comprises a coding region encoding the N-terminal region and a 3′ ribozyme, where the 3′ ribozyme is able to catalyze itself out of the RNA molecule leaving the coding region with a 3′P or 2′3′ cyclic phosphate (cP) end. In one embodiment, the 3′ ribozyme comprises an HDV ribozyme. In one embodiment, the second RNA molecule comprises a coding region encoding the C-terminal region and a 5′ ribozyme, where the 5′ ribozyme is able to catalyze itself out of the RNA molecule leaving the coding region with a 5′0H end. In one embodiment, the 5′ ribozyme comprises an HH ribozyme. In one embodiment, the additional RNA molecules each comprise a coding region encoding a protein domain, a 3′ ribozyme and a 5′ ribozyme. In one embodiment, the 3′ribozyme is an HDV ribozyme. In one embodiment, the 5′ribozyme is an HH ribozyme. In certain aspects, the 3′ribozyme is able to catalyze itself out of the RNA molecule and the 5′ribozyme is able to catalyze itself out of the RNA molecule leaving the coding region with a 5′0H and a 3′P or 2′3′ cP end. In one embodiment, the additional RNA molecules each comprise a coding region encoding a protein domain, a 5′ ribozyme and a 3′ ribozyme recognition sequence. In certain aspects, the 5′ribozyme is able to catalyze itself out of the RNA molecule leaving the coding region with a 5′OH end; and the 3′ribozyme recognition sequence interacts with a ribozyme to induce the splicing of the 3′ribozyme recognition sequence out of RNA molecule leaving coding region with and a 3′P or 2′3′ cP end. In one embodiment, the 3′ribozyme recognition sequence comprises a Vsvl sequence that interacts with a VS ribozyme. This technique can be used to generate RNA molecules encoding a protein with multiple repeat domains by sequentially adding coding regions encoding a repeat domain by sequentially providing a ribozyme (e.g. VS ribozyme) to interact with a 3′ ribozyme recognition sequence to generate a 3′P or 2′3′ cP end and ligating the coding region to the 5′OH end of another coding region encoding a repeat domain. In certain aspects, the sequential addition of repeat domains can be performed on a solid substrate or support, where the first RNA molecule encoding the N-terminal region is bound to the substrate or support.

In certain aspects, the multiple RNA molecules are ligated together after ribozyme-mediated generation of the 5′OH and 3′P or 2′3′ cP ends. In some instances, the RNA molecules are ligated together by an endogenous ligase that exists in the native cell or tissue in which the RNA assembly is taking place. In some instances, the method of the present invention comprises the step of adding an exogenous ligase to induce the ligation of the processed RNA molecules together. In one embodiment, the ligase is RNA 2′,3′-Cyclic Phosphate and 5′-OH (RtcB) ligase.

Compositions

In one embodiment, the present invention relates to a composition comprising one or more nucleic acid molecule encoding one or more ribozyme. In one embodiment, the present invention comprises one or more RNA molecule comprising one or more ribozyme. In some embodiments, the one or more RNA molecule comprises at least a first RNA molecule and a second RNA molecule.

In some embodiments, said one or more ribozyme of the composition is capable of spontaneously cis-cleaving from said one or more RNA molecule. In some embodiments, said one or more ribozyme is a 3′ ribozyme. In some embodiments, said 3′ ribozyme generates a 3′P or 2′3′ cP end on the remaining one or more RNA molecule after spontaneous cis-cleavage. In some embodiments, said one or more ribozyme is a 5′ ribozyme. In some embodiments, said 5′ ribozyme generates a 5′OH end on the remaining one or more RNA molecules after spontaneous cis-cleavage. In some embodiments, said 3′P or 2′3′ cP end and said 5′OH end can be ligated together.

In some embodiments, said first RNA molecule comprises a 3′ ribozyme. In some embodiments, said 3′ ribozyme is from one or more family selected from the group consisting of: Hammerhead (HH), Hepatitis Delta Virus (HDV), Varkud Satellite (VS), Twister (Twst), Sister, Twister-sister (TS), Hairpin, Hatchet and Pistol, or a variant or fragment thereof that maintains cis-cleaving functionality. In some embodiments, the 3′ ribozyme comprises an overhang of one or more nucleotides. In one embodiment, the overhang comprises a nucleotide sequence that hybridizes to a sequence upstream of said 3′ ribozyme within the first RNA molecule. In some embodiments, the overhang improves efficiency of spontaneous cis-cleavage.

In some embodiments, said second RNA molecule comprises a 5′ ribozyme. In some embodiments, said 5′ ribozyme is from one or more family selected from the group consisting of: Hammerhead (HH), Hepatitis Delta Virus (HDV), Varkud Satellite (VS), Twister (Twst), Sister, Twister-sister (TS), Hairpin, Hatchet and Pistol, or a variant or fragment thereof that maintains cis-cleaving functionality. In some embodiments, the 5′ ribozyme comprises an overhang of one or more nucleotides. In one embodiment, the overhang comprises a nucleotide sequence that hybridizes to a sequence downstream of said 5′ ribozyme within the second RNA molecule. In some embodiments, the overhang improves efficiency of spontaneous cis-cleavage.

In one embodiment, the HDV ribozyme of the composition comprises one or more selected from the group consisting of: HDV, HDV68, HDV67, HDV56, genHDV, and antiHDV, or a variant or fragment thereof. In one embodiment, HDV68 comprises the nucleic acid sequence of SEQ ID NO: 9. In one embodiment, HDV67 comprises the nucleic acid sequence of SEQ ID NO: 10. In one embodiment, HDV56 comprises the nucleic acid sequence of SEQ ID NO: 11. In one embodiment, genHDV comprises the nucleic acid sequence of SEQ ID NO: 12. In one embodiment, antiHDV comprises the nucleic acid sequence of SEQ ID NO: 13.

In one embodiment, the HH ribozyme comprises one or more nucleotides in a stem 1 overhang that hybridize with nucleotides of the sequence upstream or downstream of said HH ribozyme. In one embodiment, the number of nucleotides in the Stem 1 overhang can be 1 or more nucleotides, 2 or more nucleotides, 4 or more nucleotides, 6 or more nucleotides, 8 or more nucleotides, 10 or more nucleotide, 12 or more nucleotides, 14 or more nucleotides, 16 or more nucleotides, 18 or more nucleotides, or 20 or more nucleotides. In one embodiment, the HH ribozyme comprising one or more nucleotide stem 1 overhang comprises a nucleic acid sequence selected from the group consisting of: SEQ ID NO: 111, SEQ ID NO: 112, SEQ ID NO: 113, SEQ ID NO: 114, SEQ ID NO: 115, SEQ ID NO: 116, SEQ ID NO: 117, and SEQ ID NO: 118, wherein nucleotides designated as N correspond to nucleotides that hybridize with nucleotides of the sequence downstream of said HH ribozyme. In one embodiment, the HH ribozyme has one or more nucleotide in a stem 3 overhang. In one embodiment, the HH ribozyme has a 5 nucleotide stem 3 overhang. In one embodiment, the HH ribozyme comprises the nucleic acid sequence of SEQ ID NO: 105, wherein nucleotides designated as N correspond to nucleotides that hybridize with nucleotides of the sequence upstream of said HH ribozyme. In one embodiment, the HH ribozyme is modified in the stem 2 loop. In one embodiment, the HH ribozyme with a modified stem 2 loop comprises a nucleic acid sequence selected from the group consisting of: SEQ ID NO: 119, SEQ ID NO: 120, SEQ ID NO: 121, SEQ ID NO: 122, SEQ ID NO: 123, and SEQ ID NO: 124, wherein nucleotides designated as N correspond to nucleotides that hybridize with nucleotides of the sequence downstream of said HH ribozyme. In one embodiment, the HH ribozyme is modified in stem 1 to include a tertiary stabilizing motif (TSM). In one embodiment, the HH ribozyme is modified in the stem 2 loop and is modified in stem 1 to include a tertiary stabilizing motif (TSM). In one embodiment, the modified HH ribozyme cis-cleaves more efficiently than HH ribozyme. In one embodiment, the modified HH ribozyme is RzB. In one embodiment, RzB comprises the nucleic acid sequence of SEQ ID NO: 125, wherein nucleotides designated as N correspond to nucleotides that hybridize with nucleotides of the sequence downstream of said HH ribozyme.

In one embodiment, the Twister ribozyme comprises the nucleic acid sequence of SEQ ID NO: 32. In one embodiment, the Twister ribozyme comprises one or more nucleotide in a P1 stem overhang. In one embodiment, number of nucleotides in the P1 stem overhang can be 1 or more, 2 or more, 3 or more , 4 or more, or 5 or more. In one embodiment, the Twister ribozyme comprising one or more nucleotide P1 stem overhang comprises a nucleic acid sequence selected from the group consisting of: SEQ ID NO: 106, SEQ ID NO: 107, SEQ ID NO: 108, SEQ ID NO: 109, and SEQ ID NO: 110, wherein nucleotides designated as N correspond to nucleotides that hybridize with nucleotides of the sequence downstream of said Twister ribozyme.

In some embodiments, said one or more ribozyme of the composition is composed of first part and a second part. In some embodiments, the first part is incorporated into said one or more RNA molecule. In some embodiments, the first part is a ribozyme recognition sequence. In some embodiments, said second part is introduced separately. In some embodiments, cis-cleavage of the first part from said one or more RNA molecule only occurs if the first part and the second part are brought into contact with one another. In some embodiments, said one or more ribozyme is VS ribozyme. In one embodiment, said VS ribozyme comprises the nucleic acid sequence of SEQ ID NO: 14. In one embodiment, said first part is VS ribozyme stem loop (VS-S). In one embodiment, VS-S comprises the nucleic acid sequence of SEQ ID NO: 15. In one embodiment, said second part is the remaining portion of VS without the stem loop (VS-Rz). In one embodiment, VS-Rz comprises the nucleic acid sequence of SEQ ID NO: 16.

Ribozymes are autocatalytic RNAs which cleave in cis, to produce unique RNA 3′ and 5′ termini, as described herein. However, cis-cleaving ribozymes can be engineered to cleave in trans, such that target RNAs can be cleaved in a nucleotide specific manner, resulting in similar RNA termini. In some embodiments, the present invention comprises a composition comprising a single nucleic acid molecule encoding a single RNA molecule comprising a trans-cleaving engineered ribozyme. In one embodiment, said trans-cleaving engineered ribozyme is capable of trans-cleaving a separate RNA molecule. In one embodiment, said trans-cleaving engineered ribozyme recognizes a specific nucleic acid sequence in the separate RNA molecule. In some embodiments, the trans-cleaving engineered ribozyme targets a disease causing mutation for deletion. In some embodiment, the disease causing mutation is in an exon. In some embodiment, the disease causing mutation is in an intron. In some embodiments, the composition comprises two trans-cleaving engineered ribozymes, targeted upstream and downstream of the disease causing mutation. In some embodiments, trans-cleavage upstream and downstream of the disease causing mutation results in removal of the disease causing mutation. In some embodiments, the remaining portions of the gene are trans-spliced together after trans-cleavage of the disease causing mutation. In some embodiments, the trans-spliced gene is expressed as a functional protein.

As described herein, the 3′P or 2′3′ cP end and the 5′OH end of RNA molecules that have undergone ribozyme-mediated cleavage can be ligated together. As such, separated RNA sequences encoding separate portions of a larger full-length protein can be trans-spliced together in a scar-less manner to enable expression of the full-length protein. In one embodiment, the present invention relates to a composition comprising one or more nucleic acid molecule encoding two or more portions of a protein of interest and encoding one or more ribozyme. In one embodiment, the present invention relates to a composition comprising one or more RNA molecule encoding two or more portions protein of interest and comprising one or more ribozyme.

In one embodiment, said one or more nucleic acid molecules encoding two or more portions of a protein of interest comprise a first nucleic acid molecule encoding a first portion of a protein of interest and a second nucleic acid molecule encoding a second portion of a protein of interest. In one embodiment, said first nucleic acid comprises a first RNA molecule. In one embodiment, said second nucleic acid comprises a second RNA molecule. In one embodiment, the first RNA molecule is linked at the 3′ end to a 3′ ribozyme. In one embodiment, the second RNA molecule is linked at the 5′ end to a 5′ ribozyme. In one embodiment, upon cis-cleavage of the 3′ and 5′ ribozyme sequences, the 3′P or 2′3′ cP end of first RNA molecule is ligated to the 5′OH end of the second RNA molecule, thereby generating a single RNA molecule encoding a full-length protein of interest. In one embodiment, the full-length protein of interest functions identically to an endogenously expressed full-length protein of the same sequence.

In one embodiment, the full-length protein of interest comprises a therapeutic protein. In one embodiment, the therapeutic protein comprises one or more selected from the group consisting of, but not limited to: Utrophin, Dystrophin, Dysferlin, Myoferlin, Cystic fibrosis transmembrane conductance regulator (CFTR), Coagulation Factor VIII, Fibrocystin, Retinal-specific phospholipid-transporting ATPase (ABCA4), Otoferlin, Copper-transporting ATPase 2, MYO7A, MYO15A, CDH23, STRC, OTOG, TECTA, PCDH15, TRIOBP, MYO3A, COL11A2, LOXHD1, PTPRQ, OTOGL, MYH14, MYH9, TNC, CACNA1A, CACNA1C, CACNA1F, CACNA1H, CACNA1G, CACNA1D, CACNA1B, CACNA1S, CACNA1I, CACNA1E, ATP2A1, ATP2A2, Adcy6, FKBP12-rapamycin-binding domain and Cas9. In one embodiment, the full-length protein of interest is a recombinase. In one embodiment, the recombinase is one or more selected from the group consisting of, but not limited to: CRE recombinase, FLP recombinase. In one embodiment, the full-length protein of interest is a eukaryotic/prokaryotic antibiotic resistance gene product. In one embodiment, the eukaryotic/prokaryotic antibiotic resistance gene product is one or more selected from the group consisting of, but not limited to: ampicillin, kanamycin, blasticidin, puromycin, neomycin, and hygromycin. In certain embodiments, the full-length protein of interest is an antibody. In one embodiment, the antibody is capable of binding to a target protein of interest. In some embodiments, the antibody is an antibody fragment, synthetic antibody, nanobody, or a fragment or variant thereof that maintains the ability to bind to the target protein. In one embodiment, the full-length protein of interest comprises a synthetic repeat protein, including, but not limited to, those composing hydrogels, synthetic spider silks, and collagens. In one embodiment, the synthetic repeat protein comprises one or more selected from the group consisting of, but not limited to: Spidroin, Silk, Keratin, Collagen, Elastin, Resilin, and Squid Ring Teeth, beta-solenoid proteins, Zinc Finger Nucleases (ZFNs, and Tal effector nucleases (TALENs). In one embodiment, the full-length protein of interest comprises a toxic protein or an antiviral protein, which may inhibit generation of lentiviral particles in mammalian packing cells. In one embodiment, the toxic protein is a cell suicide gene. In one embodiment, the cell suicide gene comprises one or more selected from the group consisting of, but not limited to: diphtheria toxin A (DTA), HSV-tk, Ricin, Cholera toxin, Major Prion Protein, Pertussis toxin, Ectatomin, Conopeptides, Abrin, Verotoxin, Tetanospasmin, Botulinum toxin, pseudomonas exotoxin A, anthrax, saporin, and pokeweed antiviral protein (PAP). In one embodiment, the antiviral protein comprises one or more selected from the group consisting of, but not limited to: Interferon-induced GTP-binding protein (MxA), Myeloperoxidase (MPO), and Interferon.

N-terminal or C-terminal RNA molecules encoding a portion of a protein of interest could be subject to translation prior to ribozyme-mediated cleavage, or when expressed separately, potentially resulting in unwanted or truncated protein expression. However, translational control of protein degradation sequences can be utilized to limit this unwanted expression. In one embodiment, said one or more RNA molecule of the composition comprises a nucleic acid sequence encoding a translational control of protein degradation sequence. In one embodiment, said first RNA molecule comprises a nucleic acid sequence encoding a translational control of protein degradation sequence. In one embodiment, said second RNA molecule comprises a nucleic acid sequence encoding a translational control of protein degradation sequence. In some embodiments, said translational control of protein degradation sequences prevent partial expression of protein prior to cleavage of ribozyme sequences and splicing. In some embodiments, the translational control of protein degradation sequences comprise one or more selected from the group consisting of: a hCL1-PEST sequence, an E1A-PEST sequence, removal of the nucleic acid's poly(A) sequence, simulated translation through a poly A tail to generate a poly K tail, deletion of the ATG stop codon, silent mutations within N-terminal NTG codons, a 5′ UTR of yeast GCN4 sequence encoding four small upstream ORFs that function as translation inhibitors, a small internal fragment of a 5′ UTR of yeast GCN4 sequence. In some embodiments, the translational control of protein degradation sequences comprise one or more nucleic acid sequence selected from the group consisting of: SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 77, SEQ ID NO: 79, and SEQ ID NO: 104. In some embodiments, the translational control of protein degradation sequences comprise one or more amino acid sequence selected from the group consisting of: SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO:61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 76, SEQ ID NO: 78, and SEQ ID NO: 80.

In certain aspects, to further prevent unwanted or truncated protein expression, RNA nuclear localization signals may be useful to prevent cytosolic export and translation of un-spliced RNA molecules. In one embodiment, said one or more RNA molecule of the composition comprises a nucleic acid sequence encoding an RNA nuclear localization sequence. In one embodiment, said first RNA molecule comprises a nucleic acid sequence encoding an RNA nuclear localization sequence. In one embodiment, said second RNA molecule comprises a nucleic acid sequence encoding an RNA nuclear localization sequence. In one embodiment, said RNA nuclear localization sequences prevent cytosolic RNA export and translation of partial protein prior to cleavage of ribozyme sequences and splicing. In one embodiment, the RNA nuclear localization sequences comprise one or more nucleic acid sequence selected from the group consisting of: SEQ ID NO: 50, and SEQ ID NO: 51.

In some embodiments, the composition further comprises one or more additional RNA molecule, each additional RNA molecule comprising a coding region encoding a domain of the protein of interest; a 5′ ribozyme; and a 3′ ribozyme. In some embodiments, the system further comprises one or more additional nucleic acid molecule encoding one or more additional RNA molecule, each additional RNA molecule comprising a coding region encoding a domain of the protein of interest; a 5′ ribozyme; and a 3′ ribozyme.

In some embodiments, the composition further comprises one or more additional RNA molecule, each additional RNA molecule comprising a coding region encoding a domain of the protein of interest; a 5′ ribozyme; and a 3′ ribozyme recognition sequence. In some embodiments, the system further comprises one or more additional nucleic acid molecule encoding one or more additional RNA molecule, each additional RNA molecule comprising a coding region encoding a domain of the protein of interest; a 5′ ribozyme; and a 3′ ribozyme recognition sequence.

Pre-mRNA splicing by the spliceosome has been shown to enhance mRNA translation, either through deposition of factors which promote a pioneer round of translation or through promoting RNA processing and export to the cytoplasm. The addition of a chimeric cis-splicing intron within a transgene has also been shown to promote transgene protein expression. Thus, in certain embodiments, the addition of splice donor and splice acceptor sites recognized and cis-spliced by the spliceosome may enhance protein expression from split precursor RNA molecules. In one embodiment, the composition comprises one or more RNA molecule comprising a splice donor or a splice acceptor sequence. In one embodiment, said first RNA molecule of the composition comprises splice donor sequence. In one embodiment, said splice donor sequence is linked to the 3′ end of the first RNA molecule following the ribozyme sequence. In one embodiment, said second RNA molecule of the composition comprises a splice acceptor sequence. In one embodiment, said splice acceptor sequence is linked to the 5′ end of the second RNA molecule before the ribozyme sequence. In one embodiment, inclusion of the splice donor and splice acceptor sequences enhances protein expression following ribozyme-mediated trans-splicing.

Ribozyme mediated trans-splicing and expression of multiple different functional proteins at the same time may also be possible due to the three open reading frames in which proteins are translated. By harnessing this feature, functional proteins can be generated using trans-splicing of RNAs which are in three different incompatible open reading frames. In one embodiment, the composition of the present invention comprises at least four nucleic acid molecules comprising at least two pairs of nucleic acid molecules. In one embodiment, each pair of nucleic acid molecules encodes at least two portions of a protein of interest and encodes at least two ribozymes. In one embodiment, the composition comprises at least four RNA molecules comprising at least two pairs of RNA molecules. In one embodiment, each pair of RNA molecules encodes at least two portions of a protein of interest and comprises at least two ribozymes

In one embodiment, said at least two pairs of RNA molecules comprises a first pair of RNA molecules and second pair of RNA molecules. In one embodiment, the first pair of RNA molecules comprises a first RNA molecule and a second RNA molecule. In one embodiment, the second pair of RNA molecules comprises a third RNA molecule and fourth RNA molecule. In some embodiments, said third RNA molecule and said fourth RNA molecule have different open reading frame the first RNA molecule and the second RNA molecule, such that, upon spontaneous cis-cleavage, ligation of either the first RNA molecule or the second RNA molecule with either the third RNA molecule or fourth RNA molecule cannot translate a full-length functional protein product.

In one embodiment, said at least two pairs of RNA molecules further comprises a third pair of RNA molecules. In one embodiment, the third pair of RNA molecules comprises a fifth RNA molecule and a sixth RNA molecule. In some embodiments, said fifth RNA molecule and said sixth RNA molecule have different open reading frame the first pair of RNA molecules and the second pair of RNA molecules, such that, upon spontaneous cis-cleavage, only ligation of the first pair, second pair or third pair of RNA molecules can translate a full-length functional protein product.

Ribozyme-mediated trans-splicing between two independent RNAs can occur when one RNA contains a 3′ ribozyme and another contains 5′ ribozyme, as described herein. However, when transcribed in cis within the same RNA molecule, two ribozymes can mediate their own scar-less removal. This approach similarly generates two independent RNAs with 3′-P and 5′ OH termini, which can be subject to trans-splicing and translation in cells. Inclusion of a cargo sequence between said 3′ and 5′ ribozymes also produces the possibility of generating a circularized RNA molecule upon ligation.

In one embodiment, the present invention relates to a composition comprising a single nucleic acid molecule encoding two or more portions of a protein of interest and encoding one or more ribozyme. In one embodiment, the present invention relates to a composition comprising a single RNA molecule encoding two or more portions protein of interest and comprising one or more ribozyme.

In one embodiment, said single nucleic acid molecule encodes a first portion of RNA, a synthetic intron, and a second portion of RNA. In one embodiment, the synthetic intron comprises a 5′ ribozyme and a 3′ ribozyme. In one embodiment, said first portion of RNA encodes a first portion of a protein of interest. In one embodiment, said second portion of RNA encodes a second portion of a protein of interest. In one embodiment, said single nucleic acid comprises a sequence linked in the order: (first portion of RNA encoding first portion of protein of interest)-(5′ ribozyme of synthetic intron)-(3′ ribozyme of synthetic intron)-(second portion of RNA encoding second portion of protein of interest). In one embodiment, said first portion of the protein of interest is the N-terminal portion of GFP. In one embodiment, the 5′ ribozyme of the synthetic intron comprises HDV. In one embodiment, the first portion of RNA and the 5′ ribozyme of the synthetic intron comprise the nucleic acid sequence of SEQ ID NO: 127, wherein lowercase letters designate the 5′ ribozyme sequence and uppercase letters designate the sequence encoding the N-terminal portion of GFP (See Example 4, “GFP with internal synthetic ribozyme intron with and without cargo”). In one embodiment, said second portion of the protein of interest is the C-terminal portion of GFP. In one embodiment, said 3′ ribozyme of the synthetic intron comprises HH. In one embodiment, the second portion of RNA and the 3′ ribozyme of the synthetic intron comprise the nucleic acid sequence of SEQ ID NO: 128, wherein lowercase letters designate the 3′ ribozyme sequence and uppercase letters designate the sequence encoding the C-terminal portion of GFP. (See Example 4, “GFP with internal synthetic ribozyme intron with and without cargo”).

In one embodiment, said synthetic intron comprises a cargo sequence placed between said 5′ ribozyme and said 3′ ribozyme. In one embodiment, said single nucleic acid comprises a sequence linked in the order: (first portion of RNA encoding first portion of protein of interest)-(5′ ribozyme of synthetic intron)-(cargo sequence)-(3′ ribozyme of synthetic intron)-(second portion of RNA encoding second portion of protein of interest).

In one embodiment, the 5′ ribozyme sequence of the synthetic intron does not require bilateral flanking sequences for activity. In one embodiment, circular RNA generated from the ligation of the ends of the synthetic intron comprising a 5′ ribozyme sequence that does not require bilateral flanking sequences for activity can exist in both circular and re-cleaved linear forms. In one embodiment, said ribozyme sequence is a HDV ribozyme.

In one embodiment, the 5′ ribozyme sequence of the synthetic intron does require bilateral flanking sequences for activity. In one embodiment, circular RNA generated from ligation of the ends of the synthetic intron comprising a 5′ ribozyme sequence that does require bilateral flanking sequences for activity can exist only in circular form. In one embodiment, said ribozyme sequence is a HH ribozyme.

In one embodiment, the 5′ ribozyme sequence of the synthetic intron is a ribozyme recognition sequence. In one embodiment, the ribozyme recognition sequence requires the addition of a trans-cleaving ribozyme for inducible cleavage. In one embodiment, said ribozyme recognition sequence comprises VS-S. In some embodiments, VS-S is encoded by a nucleic acid sequence comprising SEQ ID NO: 15. In one embodiment, said trans-cleaving ribozyme comprises VS-Rz. In some embodiments, VS-Rz is encoded by a nucleic acid sequence comprising SEQ ID NO: 16.

In one embodiment, self-cleavage of the 5′ ribozyme sequence and the 3′ ribozyme sequence generates three separate RNA molecules: 1) a first fragment comprising the first portion of RNA encoding a first portion of a protein of interest, 2) a second fragment comprising the synthetic intron, 3) a third fragment comprising the second portion of RNA encoding a second portion of a protein of interest. In one embodiment, the compatible ends of the second fragment are ligated to generate a circular RNA molecule comprising the synthetic intron comprising the cargo sequence. In embodiment, the first fragment and third fragment are ligated together to generate a single full-length linear RNA molecule.

In one embodiment, the cargo sequence of the synthetic intron is one or more selected from the group consisting of: a sequence encoding a therapeutic protein of interest, a CRISPR guide RNA sequence, a small RNA sequence, and a trans-cleaving ribozyme sequence. In one embodiment, said small RNA sequence comprises one or more selected from the group consisting of: microRNA (miRNA), Piwi-interacting RNA (piRNA), small interfering RNA (siRNA), small nucleolar RNA (snoRNAs), small tRNA-derived RNA (tsRNA), small rDNA-derived RNA (srRNA) and small nuclear RNA (snRNA).

In one embodiment, the single full-length linear RNA molecule encodes a full-length protein of interest. In one embodiment, the full-length protein of interest is a therapeutic protein. In one embodiment, the therapeutic protein can be, but is not limited to, one or more selected from the group consisting of: Utrophin, Dystrophin, Dysferlin, Myoferlin, Cystic fibrosis transmembrane conductance regulator (CFTR), Coagulation Factor VIII, Fibrocystin, Retinal-specific phospholipid-transporting ATPase (ABCA4), Otoferlin, Copper-transporting ATPase 2, MYO7A, MYO15A, CDH23, STRC, OTOG, TECTA, PCDH15, TRIOBP, MYO3A, COL11A2, LOXHD1, PTPRQ, OTOGL, MYH14, MYH9, TNC, CACNA1A, CACNA1C, CACNA1F, CACNA1H, CACNA1G, CACNA1D, CACNA1B, CACNA1S, CACNA1I, CACNA1E, ATP2A1, ATP2A2, Adcy6, FKBP12-rapamycin-binding domain and Cas9. In one embodiment, the full-length protein of interest is a recombinase. In one embodiment, the recombinase is one or more selected from the group consisting of, but not limited to: CRE recombinase, FLP recombinase. In one embodiment, the full-length protein of interest is a eukaryotic/prokaryotic antibiotic resistance gene product. In one embodiment, the eukaryotic/prokaryotic antibiotic resistance gene product is one or more selected from the group consisting of, but not limited to: ampicillin, kanamycin, blasticidin, puromycin, neomycin, and hygromycin. In one embodiment, the full-length protein of interest is a reporter protein. In one embodiment, the reporter protein is one or more selected from the group consisting of: green fluorescent protein (GFP), red fluorescent protein (RFP), and luciferase (Luc). In one embodiment, the reporter protein is used as a proxy indicator to assess delivery and expression of the cargo sequence. In certain embodiments, the full-length protein of interest is an antibody. In one embodiment, the antibody is capable of binding to a target protein of interest. In some embodiments, the antibody is an antibody fragment, synthetic antibody, nanobody, or a fragment or variant thereof that maintains the ability to bind to the target protein.

In certain aspects, the technology of the present invention can be used to assemble a full-length RNA virus genome. In one embodiment, said one or more nucleic acid molecule encoding one or more ribozyme of the present invention encodes one or more portion of an RNA virus genome. In one embodiment, said one or more RNA molecule comprising one or more ribozyme of the present invention comprises one or more portion of an RNA virus genome.

In one embodiment, said one or more nucleic acid molecule comprises a first nucleic acid molecule encoding a first portion of the RNA virus genome and encoding a 3′ ribozyme. In one embodiment, said one or more nucleic acid molecule comprises a second nucleic acid encoding a second portion of the RNA virus genome and encoding a 5′ ribozyme. In one embodiment, said one or more RNA molecule comprises a first RNA molecule comprising a first portion of the RNA virus genome and a 3′ ribozyme. In one embodiment, the said one or more RNA molecule comprises a second RNA molecule comprising a second portion of the RNA virus genome and a 5′ ribozyme. In one embodiment, the composition comprises a nucleic acid encoding a ligase or a ligase. In one embodiment, upon cis-cleavage of the 3′ and 5′ ribozymes, the first portion of the RNA virus genome and the second portion of the RNA virus genome are ligated together, thereby generating a full-length RNA virus genome. Exemplary RNA viruses include, but are not limited to: coronaviruses, paramyxoviruses, orthomyxoviruses, retroviruses, lentiviruses, alphaviruses, flaviviruses, rhabdoviruses, measles viruses, Newcastle disease viruses, and picornaviruses.

In some embodiments, the present invention comprises a composition comprising a nucleic acid encoding a ligase. In some embodiments, the ligase mediates ligation of the 3′P or 2′3′ cP end and the 5′0H end. In some embodiments, the ligase is RNA 2′,3′-Cyclic Phosphate and 5′-OH (RtcB) ligase. In some embodiments, the RtcB ligase is from one or more domain of organism selected from the group consisting of: Eukarya, Bacteria, and Archaea. In some embodiments, the organism is selected from the group consisting of: human, E. coli, Deinococcus radiodurans, Pyrococcus horikoshii, Pyrococcus sp. ST04, and Thermococcus sp. EP. In some embodiments, the nucleic acid sequence encoding a ligase is one or more selected from the group consisting of: SEQ ID NO: 82, SEQ ID NO: 84, SEQ ID NO: 86, SEQ ID NO: 88, SEQ ID NO: 90, SEQ ID NO: 92. In some embodiments, the nucleic acid sequence encoding a ligase encodes one or more amino acid sequence selected from the group consisting of: SEQ ID NO: 81, SEQ ID NO: 83, SEQ ID NO: 85, SEQ ID NO: 87, SEQ ID NO: 89, SEQ ID NO: 91.

Nucleic Acids

In some embodiments, one or more nucleic acid of the present invention comprises a nucleic acid sequence that is substantially homologous to a nucleic acid sequence described herein. For example, in some embodiments, the nucleic acid has a degree of identity with respect to the original nucleic acid sequence of at least 60%, of at least 65%, of at least 70%, of at least 75%, of at least 80%, of at least 81%, of at least 82%, of at least 83%, of at least 84%, of at least 85%, of at least 86%, of at least 87%, of at least 88%, of at least 89%, of at least 90%, of at least 91%, of at least 92%, of at least 93%, of at least 94%, of at least 95%, of at least 96%, of at least 97%, of at least 98%, of at least 99%, or of at least 99.5%.

In some embodiments, one or more nucleic acid of the present invention comprises a nucleic acid sequence that is a portion of a nucleic acid sequence described herein. For example, in some embodiments, the nucleic acid has a length with respect to the original nucleic acid sequence of at least 60%, of at least 65%, of at least 70%, of at least 75%, of at least 80%, of at least 81%, of at least 82%, of at least 83%, of at least 84%, of at least 85%, of at least 86%, of at least 87%, of at least 88%, of at least 89%, of at least 90%, of at least 91%, of at least 92%, of at least 93%, of at least 94%, of at least 95%, of at least 96%, of at least 97%, of at least 98%, of at least 99%, or of at least 99.5%.

In some embodiments, one or more nucleic acid of the present invention comprises a nucleic acid sequence that is a portion of a nucleic acid sequence described herein, and is substantially homologous to a nucleic acid sequence described herein. For example, in some embodiments, the nucleic acid has a degree of identity with respect to the original nucleic acid sequence of at least 60%, of at least 65%, of at least 70%, of at least 75%, of at least 80%, of at least 81%, of at least 82%, of at least 83%, of at least 84%, of at least 85%, of at least 86%, of at least 87%, of at least 88%, of at least 89%, of at least 90%, of at least 91%, of at least 92%, of at least 93%, of at least 94%, of at least 95%, of at least 96%, of at least 97%, of at least 98%, of at least 99%, or of at least 99.5%. and/or has a length with respect to the original nucleic acid sequence of at least 60%, of at least 65%, of at least 70%, of at least 75%, of at least 80%, of at least 81%, of at least 82%, of at least 83%, of at least 84%, of at least 85%, of at least 86%, of at least 87%, of at least 88%, of at least 89%, of at least 90%, of at least 91%, of at least 92%, of at least 93%, of at least 94%, of at least 95%, of at least 96%, of at least 97%, of at least 98%, of at least 99%, or of at least 99.5%.

The nucleic acid of the present invention may comprise any type of nucleic acid, including, but not limited to DNA and RNA. For example, in one embodiment, the composition comprises an isolated DNA molecule, including for example, an isolated cDNA molecule, encoding a fusion protein of the invention. In one embodiment, the composition comprises an isolated RNA molecule encoding a fusion protein of the invention, or a functional fragment thereof.

The nucleic acid molecules of the present invention can be modified to improve stability in serum or in growth medium for cell cultures. Modifications can be added to enhance stability, functionality, and/or specificity and to minimize immunostimulatory properties of the nucleic acid molecule of the invention. For example, in order to enhance the stability, the 3′-residues may be stabilized against degradation, e.g., they may be selected such that they consist of purine nucleotides, particularly adenosine or guanosine nucleotides. Alternatively, substitution of pyrimidine nucleotides by modified analogues, e.g., substitution of uridine by 2′-deoxythymidine is tolerated and does not affect function of the molecule.

In one embodiment of the present invention the nucleic acid molecule may contain at least one modified nucleotide analogue. For example, the ends may be stabilized by incorporating modified nucleotide analogues.

Non-limiting examples of nucleotide analogues include sugar- and/or backbone-modified ribonucleotides (i.e., include modifications to the phosphate-sugar backbone). For example, the phosphodiester linkages of natural RNA may be modified to include at least one of a nitrogen or sulfur heteroatom. In exemplary backbone-modified ribonucleotides the phosphoester group connecting to adjacent ribonucleotides is replaced by a modified group, e.g., of phosphothioate group. In exemplary sugar-modified ribonucleotides, the 2′ OH-group is replaced by a group selected from H, OR, R, halo, SH, SR, NH₂, NHR, NR₂ or ON, wherein R is C₁-C₆ alkyl, alkenyl or alkynyl and halo is F, Cl, Br or I.

Other examples of modifications are nucleobase-modified ribonucleotides, i.e., ribonucleotides, containing at least one non-naturally occurring nucleobase instead of a naturally occurring nucleobase. Bases may be modified to block the activity of adenosine deaminase. Exemplary modified nucleobases include, but are not limited to, uridine and/or cytidine modified at the 5-position, e.g., 5-(2-amino)propyl uridine, 5-bromo uridine; adenosine and/or guanosines modified at the 8 position, e.g., 8-bromo guanosine; deaza nucleotides, e.g., 7-deaza-adenosine; 0- and N-alkylated nucleotides, e.g., N6-methyl adenosine are suitable. It should be noted that the above modifications may be combined.

In some instances, the nucleic acid molecule comprises at least one of the following chemical modifications: 2′-H, 2′-O-methyl, or 2′-OH modification of one or more nucleotides. In certain embodiments, a nucleic acid molecule of the invention can have enhanced resistance to nucleases. For increased nuclease resistance, a nucleic acid molecule, can include, for example, 2′-modified ribose units and/or phosphorothioate linkages. For example, the 2′ hydroxyl group (OH) can be modified or replaced with a number of different “oxy” or “deoxy” substituents. For increased nuclease resistance the nucleic acid molecules of the invention can include 2′-O-methyl, 2′-fluorine, 2′-O-methoxyethyl, 2′-O-aminopropyl, 2′-amino, and/or phosphorothioate linkages. Inclusion of locked nucleic acids (LNA), ethylene nucleic acids (ENA), e.g., 2′-4′-ethylene-bridged nucleic acids, and certain nucleobase modifications such as 2-amino-A, 2-thio (e.g., 2-thio-U), G-clamp modifications, can also increase binding affinity to a target.

In one embodiment, the nucleic acid molecule includes a 2′-modified nucleotide, e.g., a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O-N-methylacetamido (2′-O-NMA). In one embodiment, the nucleic acid molecule includes at least one 2′-O-methyl-modified nucleotide, and in some embodiments, all of the nucleotides of the nucleic acid molecule include a 2′-O-methyl modification.

In certain embodiments, the nucleic acid molecule of the invention has one or more of the following properties:

Nucleic acid agents discussed herein include otherwise unmodified RNA and DNA as well as RNA and DNA that have been modified, e.g., to improve efficacy, and polymers of nucleoside surrogates. Unmodified RNA refers to a molecule in which the components of the nucleic acid, namely sugars, bases, and phosphate moieties, are the same or essentially the same as that which occur in nature, or as occur naturally in the human body. The art has referred to rare or unusual, but naturally occurring, RNAs as modified RNAs, see, e.g., Limbach et al. (Nucleic Acids Res., 1994, 22:2183-2196). Such rare or unusual RNAs, often termed modified RNAs, are typically the result of a post-transcriptional modification and are within the term unmodified RNA as used herein. Modified RNA, as used herein, refers to a molecule in which one or more of the components of the nucleic acid, namely sugars, bases, and phosphate moieties, are different from that which occur in nature, or different from that which occurs in the human body. While they are referred to as “modified RNAs” they will of course, because of the modification, include molecules that are not, strictly speaking, RNAs. Nucleoside surrogates are molecules in which the ribophosphate backbone is replaced with a non-ribophosphate construct that allows the bases to be presented in the correct spatial relationship such that hybridization is substantially similar to what is seen with a ribophosphate backbone, e.g., non-charged mimics of the ribophosphate backbone.

Modifications of the nucleic acid of the invention may be present at one or more of, a phosphate group, a sugar group, backbone, N-terminus, C-terminus, or nucleobase.

Vectors

The present invention also includes a composition comprising one or more vector in which one or more nucleic acid molecule of the present invention is inserted. In one embodiment, the vector encodes at least two RNA molecules. In one embodiment, the vector comprises at least two RNA molecules. In some embodiments, the at least two RNA molecules are encoded by the same vector. In some embodiments, the at least two RNA molecules are contained within the same vector. In one embodiment, said at least two RNA molecules comprise a first RNA molecule and a second RNA molecule.

In some embodiments, the present invention comprises at least two vectors encoding at least two RNA molecules. In some embodiments, the at least two vectors comprise at least two RNA molecules. In some embodiments, the at least two vectors encode separate RNA molecules. In some embodiments, the at least two vectors comprise separate RNA molecules. In some embodiments, the at least two separate RNA molecules comprise a first RNA molecule and a second RNA molecule. In some embodiments, the first RNA molecule is encoded by a first vector and the second RNA molecule is encoded by a second vector. In some embodiments, the first RNA molecule comprises a first vector and the second RNA molecule comprises a second vector.

In some embodiments, the present invention further comprises a vector encoding one or more additional RNA molecule. In some embodiments, the present invention further comprises one or more vector comprising one or more additional RNA molecule. In some embodiments, each additional RNA molecule comprises a coding region encoding a domain of the protein of interest; a 5′ ribozyme; and a 3′ ribozyme. In some embodiments, each additional RNA molecule comprises a coding region encoding a domain of the protein of interest; a 5′ ribozyme; and a 3′ ribozyme recognition sequence.

The art is replete with suitable vectors that are useful in the present invention. In brief summary, the expression of natural or synthetic nucleic acids encoding a fusion protein of the invention is typically achieved by operably linking a nucleic acid encoding the fusion protein of the invention or portions thereof to a promoter, and incorporating the construct into an expression vector. The vectors to be used are suitable for replication and, optionally, integration in eukaryotic cells. Typical vectors contain transcription and translation terminators, initiation sequences, and promoters useful for regulation of the expression of the desired nucleic acid sequence.

The vectors of the present invention may also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties. In another embodiment, the invention provides a gene therapy vector.

The isolated nucleic acid of the invention can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the vector may be provided to a cell in the form of a viral vector. Viral vector technology is well known in the art and is described, for example, in Sambrook et al. (2012, Molecular Cloning: A Laboratory Manual, Cold Spring Harbor Laboratory, N.Y.), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

Further, a number of additional viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art.

In one embodiment, the composition includes a vector derived from an adeno-associated virus (AAV). The term “AAV vector” means a vector derived from an adeno-associated virus serotype, including without limitation, AAV-1, AAV-2, AAV-3, AAV-4, AAV-5, AAV-6, AAV-7, AAV-8, and AAV-9. AAV vectors have become powerful gene delivery tools for the treatment of various disorders. AAV vectors possess a number of features that render them ideally suited for gene therapy, including a lack of pathogenicity, minimal immunogenicity, and the ability to transduce postmitotic cells in a stable and efficient manner. Expression of a particular gene contained within an AAV vector can be specifically targeted to one or more types of cells by choosing the appropriate combination of AAV serotype, promoter, and delivery method.

AAV vectors can have one or more of the AAV wild-type genes deleted in whole or part, preferably the rep and/or cap genes, but retain functional flanking ITR sequences. Despite the high degree of homology, the different serotypes have tropisms for different tissues. The receptor for AAV1 is unknown; however, AAV1 is known to transduce skeletal and cardiac muscle more efficiently than AAV2. Since most of the studies have been done with pseudotyped vectors in which the vector DNA flanked with AAV2 ITR is packaged into capsids of alternate serotypes, it is clear that the biological differences are related to the capsid rather than to the genomes. Recent evidence indicates that DNA expression cassettes packaged in AAV 1 capsids are at least 1 log 10 more efficient at transducing cardiomyocytes than those packaged in AAV2 capsids. In one embodiment, the viral delivery system is an adeno-associated viral delivery system. The adeno-associated virus can be of serotype 1 (AAV 1), serotype 2 (AAV2), serotype 3 (AAV3), serotype 4 (AAV4), serotype 5 (AAV5), serotype 6 (AAV6), serotype 7 (AAV7), serotype 8 (AAV8), or serotype 9 (AAV9).

Desirable AAV fragments for assembly into vectors include the cap proteins, including the vp1 , vp2, vp3 and hypervariable regions, the rep proteins, including rep 78, rep 68, rep 52, and rep 40, and the sequences encoding these proteins. These fragments may be readily utilized in a variety of vector systems and host cells. Such fragments may be used alone, in combination with other AAV serotype sequences or fragments, or in combination with elements from other AAV or non-AAV viral sequences. As used herein, artificial AAV serotypes include, without limitation, AAV with a non-naturally occurring capsid protein. Such an artificial capsid may be generated by any suitable technique, using a selected AAV sequence (e.g., a fragment of a vp1 capsid protein) in combination with heterologous sequences which may be obtained from a different selected AAV serotype, non-contiguous portions of the same AAV serotype, from a non-AAV viral source, or from a non-viral source. An artificial AAV serotype may be, without limitation, a chimeric AAV capsid, a recombinant AAV capsid, or a “humanized” AAV capsid. Thus exemplary AAVs, or artificial AAVs, suitable for expression of one or more proteins, include AAV2/8 (see U.S. Pat. No. 7,282,199), AAV2/5 (available from the National Institutes of Health), AAV2/9 (International Patent Publication No. W02005/033321), AAV2/6 (U.S. Pat. No. 6,156,303), and AAVrh8 (International Patent Publication No. W02003/042397), among others.

In one embodiment, the composition comprises a lentiviral vector to deliver one or more nucleic acid of the present invention. In one embodiment, the present invention comprises a lentiviral vector comprising one or more RNA molecule encoding one or more protein of interest. For example, vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

In certain embodiments, the vector also includes conventional control elements which are operably linked to the transgene in a manner which permits its transcription, translation and/or expression in a cell transfected with the plasmid vector or infected with the virus produced by the invention. As used herein, “operably linked” sequences include both expression control sequences that are contiguous with the gene of interest and expression control sequences that act in trans or at a distance to control the gene of interest. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters which are native, constitutive, inducible and/or tissue-specific, are known in the art and may be utilized.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

Enhancer sequences found on a vector also regulates expression of the gene contained therein. Typically, enhancers are bound with protein factors to enhance the transcription of a gene. Enhancers may be located upstream or downstream of the gene it regulates. Enhancers may also be tissue-specific to enhance transcription in a specific cell or tissue type. In one embodiment, the vector of the present invention comprises one or more enhancers to boost transcription of the gene present within the vector.

In order to assess the expression of a fusion protein of the invention, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter- driven transcription.

Proteins

In some embodiments, the present invention comprises a composition comprising a ligase. In some embodiments, the ligase mediates ligation of the 3′P or 2′3′ cP end of an RNA molecule and the 5′0H end of an RNA molecule. In some embodiments, the ligase is RNA 2′,3′-Cyclic Phosphate and 5′-OH (RtcB) ligase. In some embodiments, the RtcB ligase is from one or more domain of organism selected from the group consisting of: Eukarya, Bacteria, and Archaea. In some embodiments, the organism is selected from the group consisting of: human, E. coli, Deinococcus radiodurans, Pyrococcus horikoshii, Pyrococcus sp. ST04, and Thermococcus sp. EP. In some embodiments, the ligase comprises one or more amino acid sequence selected from the group consisting of: SEQ ID NO: 81, SEQ ID NO: 83, SEQ ID NO: 85, SEQ ID NO: 87, SEQ ID NO: 89, SEQ ID NO: 91.

In some embodiments, one or more protein of the present invention comprises an amino acid sequence that is substantially homologous to an amino acid sequence described herein. For example, in some embodiments, the protein has a degree of identity with respect to the original amino acid sequence of at least 60%, of at least 65%, of at least 70%, of at least 75%, of at least 80%, of at least 81%, of at least 82%, of at least 83%, of at least 84%, of at least 85%, of at least 86%, of at least 87%, of at least 88%, of at least 89%, of at least 90%, of at least 91%, of at least 92%, of at least 93%, of at least 94%, of at least 95%, of at least 96%, of at least 97%, of at least 98%, of at least 99%, or of at least 99.5%.

In some embodiments, one or more protein of the present invention comprises an amino acid sequence that is a portion of an amino acid sequence described herein. For example, in some embodiments, the protein has a length with respect to the original amino acid sequence of at least 60%, of at least 65%, of at least 70%, of at least 75%, of at least 80%, of at least 81%, of at least 82%, of at least 83%, of at least 84%, of at least 85%, of at least 86%, of at least 87%, of at least 88%, of at least 89%, of at least 90%, of at least 91%, of at least 92%, of at least 93%, of at least 94%, of at least 95%, of at least 96%, of at least 97%, of at least 98%, of at least 99%, or of at least 99.5%.

In some embodiments, one or more protein of the present invention comprises an amino acid sequence that is a portion of an amino acid sequence described herein, and is substantially homologous to an amino acid sequence described herein. For example, in some embodiments, the protein has a degree of identity with respect to the original amino acid sequence of at least 60%, of at least 65%, of at least 70%, of at least 75%, of at least 80%, of at least 81%, of at least 82%, of at least 83%, of at least 84%, of at least 85%, of at least 86%, of at least 87%, of at least 88%, of at least 89%, of at least 90%, of at least 91%, of at least 92%, of at least 93%, of at least 94%, of at least 95%, of at least 96%, of at least 97%, of at least 98%, of at least 99%, or of at least 99.5% and/or has a length with respect to the original amino acid sequence of at least 60%, of at least 65%, of at least 70%, of at least 75%, of at least 80%, of at least 81%, of at least 82%, of at least 83%, of at least 84%, of at least 85%, of at least 86%, of at least 87%, of at least 88%, of at least 89%, of at least 90%, of at least 91%, of at least 92%, of at least 93%, of at least 94%, of at least 95%, of at least 96%, of at least 97%, of at least 98%, of at least 99%, or of at least 99.5%.

Pharmaceutical Compositions

The invention also encompasses the use of pharmaceutical compositions of the invention or salts thereof to practice the methods of the invention. Such a pharmaceutical composition may consist of at least one nucleic acid of the invention or a salt thereof in a form suitable for administration to a subject, or the pharmaceutical composition may comprise at least one nucleic acid of the invention or a salt thereof, and one or more pharmaceutically acceptable carriers, one or more additional ingredients, or some combination of these. The nucleic acid of the invention may be present in the pharmaceutical composition in the form of a physiologically acceptable salt, such as in combination with a physiologically acceptable cation or anion, as is well known in the art.

In an embodiment, the pharmaceutical compositions useful for practicing the methods of the invention may be administered to deliver a dose of between 1 ng/kg/day and 100 mg/kg/day. In another embodiment, the pharmaceutical compositions useful for practicing the invention may be administered to deliver a dose of between 1 ng/kg/day and 500 mg/kg/day.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

Pharmaceutical compositions that are useful in the methods of the invention may be suitably developed for oral, rectal, vaginal, parenteral, topical, pulmonary, intranasal, buccal, ophthalmic, or another route of administration. A composition useful within the methods of the invention may be directly administered to the skin, or any other tissue of a mammal. Other contemplated formulations include liposomal preparations, resealed erythrocytes containing the active ingredient, and immunologically-based formulations. The route(s) of administration will be readily apparent to the skilled artisan and will depend upon any number of factors including the type and severity of the disease being treated, the type and age of the veterinary or human subject being treated, and the like.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

As used herein, a “unit dose” is a discrete amount of the pharmaceutical composition comprising a predetermined amount of the active ingredient. The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

In one embodiment, the compositions of the invention are formulated using one or more pharmaceutically acceptable excipients or carriers. In one embodiment, the pharmaceutical compositions of the invention comprise a therapeutically effective amount of a nucleic acid of the invention and a pharmaceutically acceptable carrier. Pharmaceutically acceptable carriers that are useful, include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, isotonic agents, for example, sugars, sodium chloride, or polyalcohols such as mannitol and sorbitol are included in the composition. Prolonged absorption of the injectable compositions may be brought about by including in the composition an agent that delays absorption, for example, aluminum monostearate or gelatin. In one embodiment, the pharmaceutically acceptable carrier is not DMSO alone.

Formulations may be employed in admixtures with conventional excipients, i.e., pharmaceutically acceptable organic or inorganic carrier substances suitable for oral, vaginal, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” that may be included in the pharmaceutical compositions of the invention are known in the art and described, for example in Genaro, ed. (1985, Remington's Pharmaceutical Sciences, Mack Publishing Co., Easton, Pa.), which is incorporated herein by reference.

The composition of the invention may comprise a preservative from about 0.005% to 2.0% by total weight of the composition. The preservative is used to prevent spoilage in the case of exposure to contaminants in the environment. Examples of preservatives useful in accordance with the invention included but are not limited to those selected from the group consisting of benzyl alcohol, sorbic acid, parabens, imidurea and combinations thereof. An exemplary preservative is a combination of about 0.5% to 2.0% benzyl alcohol and 0.05% to 0.5% sorbic acid.

In one embodiment, the composition includes an anti-oxidant and a chelating agent that inhibits the degradation of the nucleic acid. Exemplary antioxidants for some compounds are BHT, BHA, alpha-tocopherol and ascorbic acid in the range of about 0.01% to 0.3% and BHT in the range of 0.03% to 0.1% by weight by total weight of the composition. In one embodiment, the chelating agent is present in an amount of from 0.01% to 0.5% by weight by total weight of the composition. Exemplary chelating agents include edetate salts (e.g. disodium edetate) and citric acid in the weight range of about 0.01% to 0.20%. In some embodiments, the chelating agent is in the range of 0.02% to 0.10% by weight by total weight of the composition. The chelating agent is useful for chelating metal ions in the composition that may be detrimental to the shelf life of the formulation. While BHT and disodium edetate are exemplary antioxidants and chelating agent respectively for some compounds, other suitable and equivalent antioxidants and chelating agents may be substituted therefore as would be known to those skilled in the art.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. As used herein, an “oily” liquid is one which comprises a carbon-containing liquid molecule and which exhibits a less polar character than water. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying.

The regimen of administration may affect what constitutes an effective amount. The therapeutic formulations may be administered to the subject either prior to or after a diagnosis of disease. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a subject, include a mammal, for example a human, may be carried out using known procedures, at dosages and for periods of time effective to prevent or treat disease. An effective amount of the nucleic acid necessary to achieve a therapeutic effect may vary according to factors such as the activity of the particular nucleic acid employed; the time of administration; the rate of excretion of the nucleic acid; the duration of the treatment; other drugs, compounds or materials used in combination with the nucleic acid; the state of the disease or disorder, age, sex, weight, condition, general health and prior medical history of the subject being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a nucleic acid of the invention is from about 1 and 5,000 mg/kg of body weight/per day. One of ordinary skill in the art would be able to study the relevant factors and make the determination regarding the effective amount of the therapeutic nucleic acid without undue experimentation.

The nucleic acid may be administered to a subject as frequently as several times daily, or it may be administered less frequently, such as once a day, once a week, once every two weeks, once a month, or even less frequently, such as once every several months or even once a year or less. It is understood that the amount of nucleic acid dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose will be readily apparent to the skilled artisan and will depend upon any number of factors, such as, but not limited to, the type and severity of the disease being treated, the type and age of the animal, etc.

Actual dosage levels of the active ingredients in the pharmaceutical compositions of this invention may be varied so as to obtain an amount of the active ingredient that is effective to achieve the desired therapeutic response for a particular subject, composition, and mode of administration, without being toxic to the subject.

A medical doctor, e.g., physician or veterinarian, having ordinary skill in the art may readily determine and prescribe the effective amount of the pharmaceutical composition required. For example, the physician or veterinarian could start doses of the nucleic acid of the invention employed in the pharmaceutical composition at levels lower than that required in order to achieve the desired therapeutic effect and gradually increase the dosage until the desired effect is achieved.

In particular embodiments, it is especially advantageous to formulate the nucleic acid in dosage unit form for ease of administration and uniformity of dosage. Dosage unit form as used herein refers to physically discrete units suited as unitary dosages for the subjects to be treated; each unit containing a predetermined quantity of therapeutic nucleic acid calculated to produce the desired therapeutic effect in association with the required pharmaceutical vehicle. The dosage unit forms of the invention are dictated by and directly dependent on (a) the unique characteristics of the nucleic acid and the particular therapeutic effect to be achieved, and (b) the limitations inherent in the art of compounding/formulating such a nucleic acid for the treatment of a disease in a subject.

In one embodiment, the compositions of the invention are administered to the subject in dosages that range from one to five times per day or more. In another embodiment, the compositions of the invention are administered to the subject in range of dosages that include, but are not limited to, once every day, every two, days, every three days to once a week, and once every two weeks. It will be readily apparent to one skilled in the art that the frequency of administration of the various combination compositions of the invention will vary from subject to subject depending on many factors including, but not limited to, age, disease or disorder to be treated, gender, overall health, and other factors. Thus, the invention should not be construed to be limited to any particular dosage regime and the precise dosage and composition to be administered to any subject will be determined by the attending physical taking all other factors about the subject into account.

Compositions of the invention for administration may be in the range of from about 1 mg to about 10,000 mg, about 20 mg to about 9,500 mg, about 40 mg to about 9,000 mg, about 75 mg to about 8,500 mg, about 150 mg to about 7,500 mg, about 200 mg to about 7,000 mg, about 3050 mg to about 6,000 mg, about 500 mg to about 5,000 mg, about 750 mg to about 4,000 mg, about 1 mg to about 3,000 mg, about 10 mg to about 2,500 mg, about 20 mg to about 2,000 mg, about 25 mg to about 1,500 mg, about 50 mg to about 1,000 mg, about 75 mg to about 900 mg, about 100 mg to about 800 mg, about 250 mg to about 750 mg, about 300 mg to about 600 mg, about 400 mg to about 500 mg, and any and all whole or partial increments there between.

In some embodiments, the dose of a composition of the invention is from about 1 mg and about 2,500 mg. In some embodiments, a dose of a composition of the invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second composition (i.e., a drug used for treating the same or another disease as that treated by the compositions of the invention) as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In one embodiment, the present invention is directed to a packaged pharmaceutical composition comprising a container holding a therapeutically effective amount of a nucleic acid of the invention, alone or in combination with a second pharmaceutical agent; and instructions for using the nucleic acid to treat, prevent, or reduce one or more symptoms of a disease in a subject.

The term “container” includes any receptacle for holding the pharmaceutical composition. For example, in one embodiment, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the nucleic acid's ability to perform its intended function, e.g., treating or preventing a disease in a subject, or delivering an imaging or diagnostic agent to a subject.

Routes of administration of any of the compositions of the invention include oral, nasal, parenteral, sublingual, transdermal, transmucosal (e.g., sublingual, lingual, (trans)buccal, and (intra)nasal,), intravesical, intraduodenal, intragastrical, rectal, intra-peritoneal, subcutaneous, intramuscular, intradermal, intra-arterial, intravenous, or administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### Systems

In some embodiments, the present invention relates to systems for cis-cleavage and trans-splicing of independent RNA molecules. In some embodiments, the present invention relates to systems cis-cleavage and trans-splicing of a single RNA molecule. In some embodiments, cis-cleavage and trans-splicing of independent RNA molecules or fragments of a single RNA molecule results in a single RNA molecule encoding a full-length protein of interest, as described herein. In some embodiments, the system comprises a ligase or a nucleic acid encoding a ligase, such as RtcB, as described herein.

In one embodiment, the present invention relates to an inducible system for generating a single RNA encoding a full-length protein from two separate RNA molecules encoding a first part and a second part of the full-length protein via cis-cleavage of ribozymes and trans-splicing of the two independent RNA molecules. In some embodiments, the system comprises a ribozyme recognition sequence and a ribozyme, as described herein. In some embodiments, the system comprises a ligase or a nucleic acid encoding a ligase, as described herein.

In one embodiment, the present invention relates to a system of assembling a full-length RNA virus genome. Exemplary RNA viruses include, but are not limited to: coronaviruses, paramyxoviruses, orthomyxoviruses, retroviruses, lentiviruses, alphaviruses, flaviviruses, rhabdoviruses, measles viruses, Newcastle disease viruses, and picornaviruses. In one embodiment, the system comprises a first nucleic acid encoding a first portion of the RNA virus genome and encoding a 3′ ribozyme. In one embodiment, the system comprises a second nucleic acid encoding a second portion of the RNA virus genome and encoding a 5′ ribozyme. In one embodiment, the system comprises a first portion of the RNA virus genome and a 3′ ribozyme. In one embodiment, the system comprises a second portion of the RNA virus genome and a 5′ ribozyme. In one embodiment, the system comprises a nucleic acid encoding a ligase or a ligase. In one embodiment, upon cis-cleavage of the 3′ and 5′ ribozymes, the first portion of the RNA virus genome and the second portion of the RNA virus genome are ligated together, thereby generating a full-length RNA virus genome.

In vivo

In one embodiment, the present invention relates to a system for delivery and expression of one or more full-length protein via cis-cleavage and trans-splicing of independent RNA molecules encoding parts of the full-length protein. In some embodiments, the system allows for the delivery and expression of large proteins that exceed the package size of traditional vectors (for example, dystrophin that exceeds the packaging size of AAV vectors), synthetic repeat domain proteins whose nucleic acid constructs are difficult to synthesize in vitro (for example, synthetic spider silk), or toxic/antiviral proteins (for example, DTA). In one embodiment, the present invention comprises an AAV system for delivery and expression of one or more full-length protein of interest. In some embodiments, the system comprises a ligase or a nucleic acid encoding a ligase, as described herein.

In one embodiment, the invention comprises a lentiviral delivery system to deliver one or more nucleic acid molecule encoding one or more protein of interest. In one aspect, the lentiviral delivery system comprises (1) a packaging plasmid, (2) an envelope plasmid, and (3) a transfer plasmid. In one embodiment, the transfer plasmid encodes a first RNA molecule and a second RNA molecule.

In one embodiment, the invention comprises a dual lentiviral delivery system, comprising a first lentiviral vector and a second lentiviral vector. In one embodiment, the first lentiviral vector system comprises (1) a packaging plasmid, (2) an envelope plasmid, and (3) a first transfer plasmid. In one embodiment, the second lentiviral vector system comprises (1) a packaging plasmid, (2) an envelope plasmid, and (3) a second transfer plasmid. In one embodiment, the first transfer plasmid encodes a first RNA molecule. In one embodiment, the second transfer plasmid encodes a second RNA molecule.

In one embodiment, the packaging plasmid comprises a nucleic acid sequence encoding a gag-pol polyprotein. In one embodiment, the gag-pol polyprotein comprises catalytically dead integrase. In one embodiment, the gag-pol polyprotein comprises the D116N integrase mutation.

In one embodiment, the envelope plasmid comprises a nucleic acid sequence encoding an envelope protein. In one embodiment, the envelope plasmid comprises a nucleic acid sequence encoding an HIV envelope protein. In one embodiment, the envelope plasmid comprises a nucleic acid sequence encoding a vesicular stomatitis virus g-protein (VSV-g) envelope protein. In one embodiment, the envelope protein can be selected based on the desired cell type.

In one embodiment, the first RNA molecule of the single transfer plasmid comprises a protein coding region encoding a first portion of the protein of interest and a 3′ ribozyme. In one embodiment, the second RNA molecule of the single transfer plasmid comprises a protein coding region encoding a second portion of the protein of interest and a 5′ ribozyme. In one embodiment, the transfer plasmid comprises a 5′ long terminal repeat (LTR) sequence and a 3′ LTR sequence. In one embodiment, the 3′ LTR is a Self-inactivating (SIN) LTR. Thus, in one embodiment, the 5′ LTR comprises a U3 sequence, an R sequence and a U5 sequence and the 3′ LTR comprises an R sequence and a U5 sequence, but does not comprise a U3 sequence. In one embodiment, the 5′LTR and 3′LTR flank the sequence encoding the first portion of the protein of interest and the second portion of the protein of interest.

In one embodiment, the first RNA molecule of the first transfer plasmid comprises a protein coding region encoding a first portion of the protein of interest and a 3′ ribozyme. In one embodiment, the second RNA molecule of the second transfer plasmid comprises a protein coding region encoding a second portion of the protein of interest and a 5′ ribozyme. In one embodiment, the first and second transfer plasmids comprise a 5′ long terminal repeat (LTR) sequence and a 3′ LTR sequence. In one embodiment, the 3′ LTR is a Self-inactivating (SIN) LTR. Thus, in one embodiment, the 5′ LTR comprises a U3 sequence, an R sequence and a U5 sequence and the 3′ LTR comprises an R sequence and a U5 sequence, but does not comprise a U3 sequence. In one embodiment, the 5′LTR and 3′LTR of the first transfer plasmid flank the sequence encoding the first portion of the protein of interest and the 3′ ribozyme. In one embodiment, the 5′LTR and 3′LTR of the second transfer plasmid flank the sequence encoding the second portion of the protein of interest and the 5′ ribozyme.

In one embodiment, the packaging plasmid, the envelope plasmid, and the transfer plasmid are introduced into a cell. In one embodiment, the cell transcribes and translates the nucleic acid sequence encoding the gag-pol protein to produce the gag-pol polyprotein. In one embodiment, the cell transcribes and translates the nucleic acid sequence encoding the envelope protein to produce the envelope protein. In one embodiment, the cell transcribes the single transfer plasmid to provide the first RNA molecule and the second RNA molecule. In one embodiment, the cell transcribes the first transfer plasmid to provide the first RNA molecule and the second transfer plasmid to provide the second RNA molecule. In one embodiment, the gag-pol protein, envelope polyprotein, first RNA molecule and second RNA molecule are packaged into a viral particle. In one embodiment, the viral particles are collected from the cell media. In one embodiment, the viral particles transduce a target cell, wherein the 3′ribozyme catalyzes itself out of the first RNA molecule, thereby generating a 3′P or 2′3′ cP end, the 5′ribozyme catalyzes itself out of the second RNA molecule, thereby generating a 5′OH end, endogenous RNA 2′,3′-Cyclic Phosphate and 5′-OH (RtcB) ligase ligates the 3′P or 2′3′ cP end to the 5′OH end, thereby generating a complete RNA molecule encoding the protein of interest, and the cell translates the protein of interest.

In one embodiment, the packaging plasmid, the envelope plasmid, and the first transfer plasmid are introduced into a cell. In one embodiment, the cell transcribes and translates the nucleic acid sequence encoding the gag-pol protein to produce the gag-pol polyprotein. In one embodiment, the cell transcribes and translates the nucleic acid sequence encoding the envelope protein to produce the envelope protein. In one embodiment, the cell transcribes the first transfer plasmid to provide the first RNA molecule. In one embodiment, the gag-pol protein, envelope polyprotein, first RNA molecule are packaged into a first viral particle. In one embodiment, the first viral particles are collected from the cell media.

In one embodiment, the packaging plasmid, the envelope plasmid, and the second transfer plasmid are introduced into a cell. In one embodiment, the cell transcribes and translates the nucleic acid sequence encoding the gag-pol protein to produce the gag-pol polyprotein. In one embodiment, the cell transcribes and translates the nucleic acid sequence encoding the envelope protein to produce the envelope protein. In one embodiment, the cell transcribes the second transfer plasmid to provide the second RNA molecule. In one embodiment, the gag-pol protein, envelope polyprotein, second RNA molecule are packaged into a second viral particle. In one embodiment, the second viral particles are collected from the cell media.

In one embodiment, the first viral particle and the second viral particle transduce a target cell, wherein the 3′ribozyme catalyzes itself out of the first RNA molecule, thereby generating a 3′P or 2′3′ cP end, the 5′ribozyme catalyzes itself out of the second RNA molecule, thereby generating a 5′0H end, endogenous RNA 2′,3′-Cyclic Phosphate and 5′-OH (RtcB) ligase ligates the 3′P or 2′3′ cP end to the 5′0H end, thereby generating a complete RNA molecule encoding the protein of interest, and the cell translates the protein of interest.In one embodiment, the present invention relates to a system of preventing unwanted partial protein expression from a split precursor RNA molecule. In one embodiment, the system comprises incorporating translational control of protein degradation sequences in the split precursor RNA molecule, as described herein.

In one embodiment, the present invention relates to a system for expression of two or more proteins of interest from two or more pairs of independent RNA molecules encoding parts of the proteins of interest via cis-cleavage of ribozymes and trans-splicing of the pairs of independent RNA molecules. In one embodiment, each individual pair of independent RNA molecules has a separate reading frame, such that trans-splicing of undesired pairs does not result in translation of a full-length functional protein, as described herein. In some embodiments, the system comprises a ligase or a nucleic acid encoding a ligase, as described herein.

In one embodiment, the present invention comprises a system for delivery and expression of a full-length protein of interest and a cargo sequence. In one embodiment, said system comprises a first portion of RNA encoding a first portion of the protein of interest linked at its 3′ end to a synthetic intron and a second portion of RNA encoding a second portion of the protein of interest linked at its 5′ end to a synthetic intron. In one embodiment, said synthetic intron is flanked on either side by a 5′ ribozyme sequence and a 3′ ribozyme sequence. In one embodiment, said synthetic intron comprises a cargo sequence placed between said 5′ ribozyme sequence and 3′ ribozyme sequence. In one embodiment, self-cleavage of the 5′ ribozyme sequence and the 3′ ribozyme sequence generates three separate RNA molecules: 1) a first fragment comprising the first portion of RNA encoding a first portion of a protein of interest, 2) a second fragment comprising the synthetic intron, 3) a third fragment comprising the second portion of RNA encoding a second portion of a protein of interest. In one embodiment, the compatible ends of the second fragment are ligated to generate a circular RNA molecule comprising the synthetic intron comprising the cargo sequence. In embodiment, the first fragment and third fragment are ligated together to generate a single full-length linear RNA molecule. In one embodiment, the full-length protein of interest comprises a therapeutic protein, a reporter protein, a recombinase, an antibiotic resistance gene product, antibody, or Cas9 protein. In one embodiment, the cargo sequence comprises a therapeutic nucleic acid sequence (for example, a miRNA sequence or a CRISPR guide RNA sequence) or encodes a therapeutic protein. In some embodiments, the full-length protein of interest comprises Cas9 and the cargo sequence comprises a guide RNA sequence, thereby targeting Cas9 to a particular genomic sequence for editing. In some embodiments, the system comprises a ligase or a nucleic acid encoding a ligase, as described herein.

In one embodiment, the present invention comprises a system for gene editing, comprising one or more trans-cleaving engineered ribozymes. In some embodiments, the system comprises two trans-cleaving engineered ribozymes, targeted upstream and downstream of the disease causing mutation. In some embodiments, trans-cleavage upstream and downstream of the disease causing mutation results in removal of the disease causing mutation. In some embodiments, the remaining portions of the gene are trans-spliced together after trans-cleavage of the disease causing mutation. In some embodiments, the trans-spliced gene is expressed as a functional protein. In some embodiments, the system comprises a ligase or a nucleic acid encoding a ligase, as described herein.

In vitro

In one embodiment, the present invention comprises an in vitro system for generating an RNA molecule encoding a protein of interest. In one embodiment, the system comprises at least two RNA molecules. In one embodiment, said at least two RNA molecules comprises a first RNA molecule and a second RNA molecule.

In one embodiment, said first RNA molecule comprises a coding region encoding a first portion of the protein of interest. In one embodiment, said first RNA molecule comprises a 3′ribozyme. In one embodiment, said first RNA molecule comprises a coding region encoding a first portion of the protein of interest and a 3′ribozyme, as described herein.

In one embodiment, said second RNA molecule comprises a coding region encoding a second portion of the protein of interest. In one embodiment, said second RNA molecule comprises a 5′ribozyme. In one embodiment, said second RNA molecule comprises a coding region encoding a second portion of the protein of interest and a 5′ribozyme, as described herein.

In one embodiment, the in vitro system for generating an RNA molecule encoding a protein of interest further comprises a ligase. In one embodiment, the ligase induces the assembly of the RNA molecule from the coding region of the first RNA molecule and the coding region of the second RNA molecule. In one embodiment, the ligase is RNA 2′,3′-Cyclic Phosphate and 5′-OH (RtcB) ligase, as described herein.

In one embodiment, the present invention comprises an in vitro system for generating an RNA molecule encoding repeat domain protein of interest. In one embodiment, said system comprises a first RNA molecule, one or more additional RNA molecule, and a last RNA molecule.

In one embodiment, said first RNA molecule comprises a coding region encoding a first portion of the protein of interest. In one embodiment, said first RNA molecule comprises a 3′ribozyme. In one embodiment, said first RNA molecule comprises a coding region encoding a first portion of the protein of interest and a 3′ribozyme. In one embodiment, said 3′ ribozyme catalyzes itself out of the first RNA molecule, thereby generating a 3′P or 2′3′ cP end. In one embodiment, said first RNA molecule further comprises a 5′ tag. In one embodiment, said 5′ tag mediates attachment of said first RNA molecule to a solid support.

In one embodiment, said one or more additional RNA molecule comprises a coding region encoding a domain of the protein of interest; a 5′ ribozyme; and a 3′ ribozyme recognition sequence. In one embodiment, said 5′ ribozyme cleaves itself to generate a 5′OH end. In one embodiment, said 3′ ribozyme recognition sequence comprises a VS-S sequence, as described herein.

In one embodiment, said last RNA molecule comprises a coding region encoding a last portion of the protein of interest. In one embodiment, said last RNA molecule comprises a 5′ribozyme. In one embodiment, said last RNA molecule comprises a coding region encoding a last portion of the protein of interest and a 5′ribozyme. In one embodiment, said 5′ ribozyme cleaves itself to generate a 5′OH end.

In one embodiment, the system further comprises a ribozyme. In one embodiment, said ribozyme comprises VS-Rz, as described herein. In one embodiment, said VS-Rz recognizes VS-S, as described herein, and mediates its cleavage from the one or more additional RNA molecule. In one embodiment, said cleavage generates a 3′P or 2′3′ cP end.

In one embodiment, the system comprises a ligase. In some embodiments, the ligase ligates the 3′P or 2′3′ cP end of the first RNA molecule to the 5′OH end of the one or more additional RNA molecule. In some embodiments, the ligase ligates the 3′P or 2′3′ cP end of the one or more additional RNA molecule to the 5′OH end of the last RNA molecule. In some embodiments, the ligase ligates the 3′P or 2′3′ cP end of the first RNA molecule to the 5′OH end of the one or more additional RNA molecule, and ligates the 3′P or 2′3′ cP end of the one or more additional RNA molecule to the 5′OH end of the last RNA molecule, thereby generating a complete RNA molecule encoding an N-terminal domain, one or more additional domain, and a C-terminal domain. In some embodiments, the ligase is RNA 2′,3′-Cyclic Phosphate and 5′-OH (RtcB) ligase, as described herein.

### Methods

In some embodiments, the present invention relates to methods of cis-cleavage and trans-splicing of independent RNA molecules. In some embodiments, the present invention relates to methods of cis-cleavage and trans-splicing of a single RNA molecule. In some embodiments, cis-cleavage and trans-splicing of independent RNA molecules or fragments of a single RNA molecule results in a single RNA molecule encoding a full-length protein of interest, as described herein. In some embodiments, the method comprises administering ligase or a nucleic acid encoding a ligase, as described herein.

In one embodiment, the present invention relates to an inducible method for generating a single RNA encoding a full-length protein from two separate RNA molecules encoding a first part and a second part of the full-length protein via cis-cleavage of ribozymes and trans-splicing of the two independent RNA molecules. In some embodiments, the method comprises a ribozyme recognition sequence and a ribozyme, as described herein. In some embodiments, the method comprises administering ligase or a nucleic acid encoding a ligase, as described herein.

In vivo

In one embodiment, the present invention comprises a method of generating an RNA molecule encoding a protein of interest. In some embodiments, the method comprises administering at least two nucleic acid molecules to a cell or tissue. In one embodiment, the at least two nucleic acid molecules comprise a first RNA molecule and a second RNA molecule. In some embodiments, the at least two nucleic acid molecules encode a first RNA molecule and a second RNA molecule.

In one embodiment, said first RNA molecule comprises a coding region encoding a first portion of the protein of interest. In one embodiment, said first RNA molecule comprises a 3′ribozyme. In one embodiment, said first RNA molecule comprises a coding region encoding a first portion of the protein of interest and a 3′ribozyme. In one embodiment, said 3′ribozyme catalyzes itself out of the first RNA molecule, thereby generating a 3′P or 2′3′ cP end. In one embodiment, the 3′ ribozyme is a member of the HDV family of ribozymes

In one embodiment, said second RNA molecule comprises a coding region encoding a second portion of the protein of interest. In one embodiment, said second RNA molecule comprises a 5′ribozyme. In one embodiment, said second RNA molecule comprises a coding region encoding a second portion of the protein of interest and a 5′ribozyme. In one embodiment, said 5′ribozyme catalyzes itself out of the second RNA molecule, thereby generating a 5′OH end. In one embodiment, the 5′ ribozyme is a member of the HH family of ribozymes.

In one embodiment, said 3′P or 2′3′ cP end is ligated to the 5′OH end to form an RNA molecule comprising the coding region of the first RNA molecule and the coding region of the second RNA molecule.

In one embodiment, the method comprises administering to the cell or tissue one or more additional nucleic acid molecules encoding one or more additional RNA molecules, each additional RNA molecule comprising a coding region encoding a domain of the protein of interest; a 5′ ribozyme; and a 3′ ribozyme.

In one embodiment, the method comprises administering to the cell or tissue one or more additional nucleic acid molecules encoding one or more additional RNA molecules, each additional RNA molecule comprising a coding region encoding a domain of the protein of interest; a 5′ ribozyme; and a 3′ ribozyme recognition sequence. In one embodiment, the 3′ ribozyme recognition sequence comprises VS-S. In one embodiment, the ribozyme is VS.

In one embodiment, the method comprises administering to the cell or tissue one or more selected from the group consisting of: a nucleic acid molecule encoding a ligase and a ligase. In one embodiment, the ligase induces the assembly of the RNA molecule from the coding region of the first RNA molecule and the coding region of the second RNA molecule. In one embodiment, the ligase is RNA 2′,3′-Cyclic Phosphate and 5′-OH (RtcB) ligase.

In some embodiments, the method comprises administering at least one AAV vector encoding a first RNA molecule comprising a protein coding region encoding a first portion of the protein of interest and a 3′ ribozyme, and a second RNA molecule comprising a protein coding region encoding a second portion of the protein of interest and a 5′ ribozyme to a cell or tissue. In some embodiments, the method comprises administering ligase or a nucleic acid encoding a ligase, as described herein.

In some embodiments, the method comprises administering at least two AAV vectors, comprising a first AAV vector and a second AAV vector. In one embodiment, the first AAV vector encodes a first RNA molecule comprising a protein coding region encoding a first portion of the protein of interest and a 3′ ribozyme. In one embodiment, the second AAV vector encodes a second RNA molecule comprising a protein coding region encoding a second portion of the protein of interest and a 5′ ribozyme to a cell or tissue. In some embodiments, the method comprises administering ligase or a nucleic acid encoding a ligase, as described herein.

In some embodiments, the method comprises administering at least one lentiviral vector, encoding a first RNA molecule comprising a protein coding region encoding a first portion of the protein of interest and a 3′ ribozyme, and a second RNA molecule comprising a protein coding region encoding a second portion of the protein of interest and a 5′ ribozyme to a cell or tissue. In some embodiments, the method comprises administering ligase or a nucleic acid encoding a ligase, as described herein.

In some embodiments, the method comprises administering at least two lentiviral vectors, comprising a first lentiviral vector and a second lentiviral vector. In one embodiment, the first lentiviral vector encodes a first RNA molecule comprising a protein coding region encoding a first portion of the protein of interest and a 3′ ribozyme. In one embodiment, the second lentiviral vector encodes a second RNA molecule comprising a protein coding region encoding a second portion of the protein of interest and a 5′ ribozyme to a cell or tissue. In some embodiments, the method comprises administering ligase or a nucleic acid encoding a ligase, as described herein.

In some embodiments, the method comprises administering at least one lentiviral vector delivery system to provide a first RNA molecule comprising a protein coding region encoding a first portion of the protein of interest and a 3′ ribozyme, and a second RNA molecule comprising a protein coding region encoding a second portion of the protein of interest and a 5′ ribozyme to a cell or tissue. In some embodiments, the method comprises administering ligase or a nucleic acid encoding a ligase, as described herein.

In some embodiments, the method comprises administering at least two lentiviral vector delivery systems, comprising a first lentiviral vector delivery system and a second lentiviral vector delivery system. In one embodiment, the first lentiviral vector delivery system provides a first RNA molecule comprising a protein coding region encoding a first portion of the protein of interest and a 3′ ribozyme. In one embodiment, the second lentiviral vector delivery system provides a second RNA molecule comprising a protein coding region encoding a second portion of the protein of interest and a 5′ ribozyme to a cell or tissue. In some embodiments, the method comprises administering ligase or a nucleic acid encoding a ligase, as described herein.

In some embodiments, the method comprises administering two or more delivery vehicles selected from the group consisting of: an AAV vector, a lentiviral vector, a lentiviral vector delivery system, or a combination thereof. In one embodiment, said two or more delivery vehicles comprises a first delivery vehicle and a second delivery vehicle. In one embodiment, the first delivery vehicle provides a first RNA molecule comprising a protein coding region encoding a first portion of the protein of interest and a 3′ ribozyme. In one embodiment, the second delivery vehicle provides a second RNA molecule comprising a protein coding region encoding a second portion of the protein of interest and a 5′ ribozyme to a cell or tissue. In some embodiments, the method comprises administering ligase or a nucleic acid encoding a ligase, as described herein.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2012, Molecular Cloning: A Laboratory Manual, Cold Spring Harbor Laboratory, N.Y.). An exemplary method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

In one embodiment, the present invention relates to a method of expressing two or more proteins of interest from two or more pairs of independent RNA molecules encoding parts of the proteins of interest via cis-cleavage of ribozymes and trans-splicing of the pairs of independent RNA molecules. In one embodiment, the method comprises administering one, two, or three pairs of nucleic acid molecules encoding or comprising RNA molecules, wherein each individual pair of independent RNA molecules has a separate reading frame, such that trans-splicing of undesired pairs does not result in translation of a full-length functional protein. In one embodiment, the method further comprises administering to the cell or tissue one or more selected from the group consisting of: a nucleic acid molecule encoding a ligase and a ligase. In one embodiment, the ligase is RNA 2′,3′-Cyclic Phosphate and 5′-OH (RtcB) ligase, as described herein.

In one embodiment, the present invention comprises a method of delivery and expression of a full-length protein of interest and a cargo sequence. In one embodiment, said method comprises administering to a cell or tissue a first portion of RNA encoding a first portion of the protein of interest linked at its 3′ end to a synthetic intron and a second portion of RNA encoding a second portion of the protein of interest linked at its 5′ end to a synthetic intron. In one embodiment, said synthetic intron is flanked on either side by a 5′ ribozyme sequence and a 3′ ribozyme sequence. In one embodiment, said synthetic intron comprises a cargo sequence placed between said 5′ ribozyme sequence and 3′ ribozyme sequence. In one embodiment, self-cleavage of the 5′ ribozyme sequence and the 3′ ribozyme sequence generates three separate RNA molecules: 1) a first fragment comprising the first portion of RNA encoding a first portion of a protein of interest, 2) a second fragment comprising the synthetic intron, 3) a third fragment comprising the second portion of RNA encoding a second portion of a protein of interest. In one embodiment, the compatible ends of the second fragment are ligated to generate a circular RNA molecule comprising the synthetic intron comprising the cargo sequence. In embodiment, the first fragment and third fragment are ligated together to generate a single full-length linear RNA molecule. In one embodiment, the full-length protein of interest comprises a therapeutic protein, a reporter protein, a recombinase, an antibiotic resistance gene product, antibody, or Cas9 protein. In one embodiment, the cargo sequence comprises a therapeutic nucleic acid sequence (for example, an miRNA sequence or a CRISPR guide RNA sequence) or encodes a therapeutic protein. In some embodiments, the full-length protein of interest comprises Cas9 and the cargo sequence comprises a guide RNA sequence, thereby targeting Cas9 to a particular genomic sequence for editing. In some embodiments, the method comprises administering to the cell or tissue a ligase or a nucleic acid encoding a ligase, as described herein.

In one embodiment, the present invention comprises a method of gene editing, comprising one or more trans-cleaving engineered ribozymes. In some embodiments, the method comprises administering a first trans-cleaving engineered ribozyme and a second trans-cleaving engineered ribozyme, wherein the first trans-cleaving engineered ribozyme targets upstream and the second trans-cleaving engineered ribozyme downstream of a disease causing mutation. In some embodiments, trans-cleavage upstream and downstream of the disease causing mutation results in removal of the disease causing mutation. In some embodiments, the remaining portions of the gene are trans-spliced together after trans-cleavage of the disease causing mutation. In some embodiments, the trans-spliced gene is expressed as a functional protein.

In one embodiment, the present invention relates to in vivo methods of assembling a full-length RNA virus genome. Exemplary RNA viruses include, but are not limited to: coronaviruses, paramyxoviruses, orthomyxoviruses, retroviruses, lentiviruses, alphaviruses, flaviviruses, rhabdoviruses, measles viruses, Newcastle disease viruses, and picornaviruses. In one embodiment, the method comprises administering to a cell or tissue a first nucleic acid encoding a first portion of the RNA virus genome and encoding a 3′ ribozyme. In one embodiment, the method comprises administering to the cell or tissue a second nucleic acid encoding a second portion of the RNA virus genome and encoding a 5′ ribozyme. In one embodiment, the method comprises administering to the cell or tissue a first RNA molecule comprising a first portion of the RNA virus genome and a 3′ ribozyme. In one embodiment, the method comprises administering to the cell or tissue a second RNA molecule comprising a second portion of the RNA virus genome and a 5′ ribozyme. In one embodiment, the method comprises administering to the cell or tissue a nucleic acid encoding a ligase or a ligase, as described herein. In one embodiment, upon cis-cleavage of the 3′ and 5′ ribozymes, the first portion of the RNA virus genome and the second portion of the RNA virus genome are ligated together, thereby generating a full-length RNA virus genome.

In vitro

In one embodiment, the present invention comprises an in vitro method of generating an RNA molecule encoding a protein of interest. In one embodiment, the method comprises the step of providing at least two RNA molecules. In one embodiment, said step comprises providing a first RNA molecule and a second RNA molecule.

In one embodiment, said first RNA molecule comprises a coding region encoding a first portion of the protein of interest. In one embodiment, said first RNA molecule comprises a 3′ribozyme. In one embodiment, said first RNA molecule comprises a coding region encoding a first portion of the protein of interest and a 3′ribozyme.

In one embodiment, said second RNA molecule comprises a coding region encoding a second portion of the protein of interest. In one embodiment, said second RNA molecule comprises a 5′ribozyme. In one embodiment, said second RNA molecule comprises a coding region encoding a second portion of the protein of interest and a 5′ribozyme.

In one embodiment, the in vitro method of generating an RNA molecule encoding a protein of interest further comprises providing a ligase. In one embodiment, the ligase induces the assembly of the RNA molecule from the coding region of the first RNA molecule and the coding region of the second RNA molecule. In one embodiment, the ligase is RNA 2′,3′-Cyclic Phosphate and 5′-OH (RtcB) ligase, as described herein.

In one embodiment, the present invention comprises an in vitro method of generating an RNA molecule encoding a multi-domain protein of interest. In one embodiment, the method comprises the steps of: a) providing a first RNA molecule, b) providing one or more additional RNA molecule, c) providing a ribozyme, and d) providing a last RNA molecule.

In one embodiment, said first RNA molecule of step a) comprises a coding region encoding a first portion of the protein of interest. In one embodiment, said first RNA molecule comprises a 3′ribozyme. In one embodiment, said first RNA molecule comprises a coding region encoding a first portion of the protein of interest and a 3′ribozyme. In one embodiment, said 3′ ribozyme catalyzes itself out of the first RNA molecule, thereby generating a 3′P or 2′3′ cP end. In one embodiment, said first RNA molecule further comprises a 5′ tag. In one embodiment, said 5′ tag mediates attachment of said first RNA molecule to a solid support.

In one embodiment, said one or more additional RNA molecule of step b) comprises a coding region encoding a domain of the protein of interest; a 5′ ribozyme; and a 3′ ribozyme recognition sequence. In one embodiment, said 5′ ribozyme cleaves itself to generate a 5′0H end. In one embodiment, a ligase is provided to catalyze ligation of the first RNA molecule to the one or more additional RNA molecule. In one embodiment, the ligase is RNA 2′,3′-Cyclic Phosphate and 5′-OH (RtcB) ligase, as described herein. In one embodiment, said 3′ ribozyme recognition sequence comprises a VS-S sequence, as described herein.

In one embodiment, said ribozyme of step c) comprises VS-Rz, as described herein. In one embodiment, said VS-Rz recognizes VS-S, and mediates its cleavage from the one or more additional RNA molecule. In one embodiment, said cleavage generates a 3′P or 2′3′ cP end. In one embodiment, steps b) through c) are repeated at least one time to generate an RNA molecule encoding a plurality of domains. In one embodiment, said VS-Rz is removed prior to repeating step b).

In one embodiment, said last RNA molecule of step d) comprises a coding region encoding a last portion of the protein of interest. In one embodiment, said last RNA molecule comprises a 5′ribozyme. In one embodiment, said last RNA molecule comprises a coding region encoding a last portion of the protein of interest and a 5′ribozyme. In one embodiment, said 5′ ribozyme catalyzes itself out of the last RNA molecule, thereby generating a 5′0H end. In one embodiment, a ligase is provided to catalyze ligation of the one or more additional RNA molecule to the last RNA molecule, thereby generating a complete RNA molecule encoding an N-terminal domain, one or more additional domain, and a C-terminal domain. In one embodiment, the ligase is RNA 2′,3′-Cyclic Phosphate and 5′-OH (RtcB) ligase, as described herein.

Any RNA molecule of the present disclosure may be transcribed in vitro from template DNA, referred to as an “in vitro transcription template.” The source of the DNA can be, for example, genomic DNA, plasmid DNA, phage DNA, cDNA, synthetic DNA sequence or any other appropriate source of DNA. In some embodiments, an in vitro transcription template encodes a 5′ untranslated (UTR) region, contains an open reading frame, and encodes a 3′ UTR and a polyA tail. The particular nucleic acid sequence composition and length of an in vitro transcription template will depend on the mRNA encoded by the template.

In one embodiment, the 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the gene of interest. Alternatively, UTR sequences that are not endogenous to the gene of interest can be added by incorporating the UTR sequences into the forward and reverse primers or by any other modifications of the template. The use of UTR sequences that are not endogenous to the gene of interest can be useful for modifying the stability and/or translation efficiency of the RNA. For example, it is known that AU-rich elements in 3′ UTR sequences can decrease the stability of mRNA. Therefore, 3′ UTRs can be selected or designed to increase the stability of the transcribed RNA based on properties of UTRs that are well known in the art.

In one embodiment, the 5′ UTR can contain the Kozak sequence of the endogenous gene. Alternatively, when a 5′ UTR that is not endogenous to the gene of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many mRNAs is known in the art. In other embodiments the 5′ UTR can be derived from an RNA virus whose RNA genome is stable in cells. In other embodiments various nucleotide analogues can be used in the 3′ or 5′ UTR to impede exonuclease degradation of the mRNA.

To enable synthesis of RNA from a DNA template, a promoter of transcription should be attached to the DNA template upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In one embodiment, the promoter is a T7 RNA polymerase promoter, as described elsewhere herein. Other useful promoters include, but are not limited to, T3 and SP6 RNA polymerase promoters. Consensus nucleotide sequences for T7, T3 and SP6 promoters are known in the art.

In one embodiment, the mRNA has both a cap on the 5′ end and a 3′ poly(A) tail which determine ribosome binding, initiation of translation and stability of mRNA in the cell. On a circular DNA template, for instance, plasmid DNA, RNA polymerase produces a long concatameric product, which is not suitable for expression in eukaryotic cells. The transcription of plasmid DNA linearized at the end of the 3′ UTR results in normal sized mRNA, which is effective in eukaryotic transfection when it is polyadenylated after transcription.

On a linear DNA template, phage T7 RNA polymerase can extend the 3′ end of the transcript beyond the last base of the template (Schenborn and Mierendorf, Nuc Acids Res., 13:6223-36 (1985); Nacheva and Berzal-Herranz, Eur. J. Biochem., 270:1485-65 (2003)).

The conventional method of integration of polyA/T stretches into a DNA template is molecular cloning. However, polyA/T sequence integrated into plasmid DNA can cause plasmid instability, which can be ameliorated through the use of recombination incompetent bacterial cells for plasmid propagation.

Poly(A) tails of RNAs can be further extended following in vitro transcription with the use of a poly(A) polymerase, such as E. coli polyA polymerase (E-PAP) or yeast polyA polymerase. In one embodiment, increasing the length of a poly(A) tail from 100 nucleotides to between 300 and 400 nucleotides results in about a two-fold increase in the translation efficiency of the RNA. Additionally, the attachment of different chemical groups to the 3′ end can increase mRNA stability. Such attachment can contain modified/artificial nucleotides, aptamers and other compounds. For example, ATP analogs can be incorporated into the poly(A) tail using poly(A) polymerase. ATP analogs can further increase the stability of the RNA. 5′ caps also provide stability to mRNA molecules. In one embodiment,

RNAs produced by the methods to include a 5′ cap1 structure. Such cap1 structure can be generated using Vaccinia capping enzyme and 2′-O-methyltransferase enzymes (CellScript, Madison, Wis.). Alternatively, 5′ cap is provided using techniques known in the art and described herein (Cougot, et al., Trends in Biochem. Sci., 29:436-444 (2001); Stepinski, et al., RNA, 7:1468-95 (2001); Elango, et al., Biochim. Biophys. Res. Commun., 330:958-966 (2005)).

Certain embodiments of the invention may make use of solid supports comprised of an inert substrate or matrix (e.g. glass slides, polymer beads etc.) which has been functionalized, for example by application of a layer or coating of an intermediate material comprising reactive groups which permit covalent attachment to biomolecules, such as polynucleotides. Examples of such supports include, but are not limited to, polyacrylamide hydrogels supported on an inert substrate such as glass, particularly polyacrylamide hydrogels as described in WO 2005/065814 and US 2008/0280773, the contents of which are incorporated herein in their entirety by reference. In such embodiments, the biomolecules (e.g. polynucleotides) may be directly covalently attached to the intermediate material (e.g. the hydrogel) but the intermediate material may itself be non-covalently attached to the substrate or matrix (e.g. the glass substrate). The term “covalent attachment to a solid support” is to be interpreted accordingly as encompassing this type of arrangement.

As will be appreciated by those in the art, the number of possible substrates is very large. Possible substrates include, but are not limited to, glass and modified or functionalized glass, plastics (including acrylics, polystyrene and copolymers of styrene and other materials, polypropylene, polyethylene, polybutylene, polyurethanes, Teflon™, etc.), polysaccharides, nylon or nitrocellulose, ceramics, resins, silica or silica-based materials including silicon and modified silicon, carbon, metals, inorganic glasses, plastics, optical fiber bundles, and a variety of other polymers.

In some embodiments, the solid support comprises microspheres or beads. Suitable bead compositions include, but are not limited to, plastics, ceramics, glass, polystyrene, methylstyrene, acrylic polymers, paramagnetic materials, thoria sol, carbon graphite, titanium dioxide, latex or cross-linked dextrans such as Sepharose, cellulose, nylon, cross-linked micelles and teflon, as well as any other materials outlined herein for solid supports may all be used. “Microsphere Detection Guide” from Bangs Laboratories, Fishers Ind. is a helpful guide. In certain embodiments, the microspheres are magnetic microspheres or beads.

The beads need not be spherical; irregular particles may be used. Alternatively, or additionally, the beads may be porous. The bead sizes range from nanometers, i.e. 100 nm, to millimeters, i.e. 1 mm, with beads from about 0.2 micron to about 200 microns being preferred, and from about 0.5 to about 5 micron being particularly preferred, although in some embodiments smaller or larger beads may be used.

In one embodiment, the present invention relates to in vitro methods of assembling a full-length RNA virus genome. Exemplary RNA viruses include, but are not limited to: coronaviruses, paramyxoviruses, orthomyxoviruses, retroviruses, lentiviruses, alphaviruses, flaviviruses, rhabdoviruses, measles viruses, Newcastle disease viruses, and picornaviruses. In one embodiment, the method comprises providing a first RNA molecule comprising a first portion of the RNA virus genome and a 3′ ribozyme. In one embodiment, the method comprises providing a second RNA molecule comprising a second portion of the RNA virus genome and a 5′ ribozyme. In one embodiment, upon cis-cleavage of the 3′ and 5′ ribozymes, as described herein, the first portion of the RNA virus genome and the second portion of the RNA virus genome have compatible termini for ligation. In one embodiment, the method comprises contacting the first RNA molecule and the second RNA molecule with a ligase, as described herein, thereby generating a full-length RNA virus genome.

Treatment and Use

The present invention provides methods of treating, reducing the symptoms of, and/or reducing the risk of developing a disease or disorder in a subject. For example, in one embodiment, methods of the invention of treat, reduce the symptoms of, and/or reduce the risk of developing a disease or disorder in a mammal. In one embodiment, the methods of the invention of treat, reduce the symptoms of, and/or reduce the risk of developing a disease or disorder in a plant. In one embodiment, the methods of the invention of treat, reduce the symptoms of, and/or reduce the risk of developing a disease or disorder in a yeast organism.

In one embodiment, the subject is a cell. In one embodiment, the cell is a prokaryotic cell or eukaryotic cell. In one embodiment, the cell is a eukaryotic cell. In one embodiment, the cell is a plants, animals, or fungi cell. In one embodiment, the cell is a plant cell. In one embodiment, the cell is an animal cell. In one embodiment, the cell is a yeast cell.

In one embodiment, the subject is a mammal. For example, in one embodiment, the subject is a human, non-human primate, dog, cat, horse, cow, goat, sheep, rabbit, pig, rat, or mouse. In one embodiment, the subject is a non-mammalian subject. For example, in one embodiment, the subject is a zebrafish, fruit fly, or roundworm.

In one embodiment, the disease or disorder is caused by an absent or defective protein, the nucleic acid sequence of which exceeds the packaging size of a viral vector. Thus, in one embodiment, the disease or disorder may treated, reduced, or the risk can be reduced using the compositions, systems and methods of the present invention. Thus, in one embodiment, the method comprises administering to the subject one or more composition of the present invention. Further, in one embodiment, the method comprises utilizing one or more system of the present invention to treat, reduce the symptoms of, and/or reduce the risk of developing a disease or disorder in a subject.

In one embodiment, the disease or disorder is one or more selected from the group consisting of: Duchenne Muscular Dystrophy, autosomal recessive polycystic kidney disease, Hemophilia A, Stargardt macular degeneration, limb-girdle muscular dystrophies , DFNB9, neurosensory nonsyndromic recessive deafness, Cystic Fibrosis, Wilson Disease, Miyoshi Muscular Dystrophy and Deafness, Autosomal Recessive 9, Usher Syndrome, Type I and Deafness, Autosomal Recessive 2, Deafness, Autosomal Recessive 3 and Nonsyndromic Hearing Loss, Usher syndrome type I, autosomal recessive deafness-16 (DFNB16), Meniere's disease (MD), Deafness, Autosomal Dominant 12 and Deafness, Autosomal Recessive 21, Usher syndrome Type 1F (USH1F) and DFNB23, Deafness, Autosomal Recessive 28 and Nonsyndromic Hearing Loss, Deafness, Autosomal Recessive 30 and Nonsyndromic Hearing Loss, Otospondylomegaepiphyseal Dysplasia, Autosomal Recessive and Otospondylomegaepiphyseal Dysplasia, Autosomal Dominant, Deafness, Autosomal Recessive 77 and Autosomal Recessive Non-Syndromic Sensorineural Deafness Type Dfnb, autosomal-recessive nonsyndromic hearing impairment DFNB84, Deafness, Autosomal Recessive 84B and Rare Genetic Deafness, Peripheral Neuropathy, Myopathy, Hoarseness, And Hearing Loss and Deafness, Autosomal Dominant 4A, congenital thrombocytopenia, sensory hearing loss, DFNA56, HXB, deafness, autosomal dominant 56, hexabrachion , epileptic encephalopathy, Timothy Syndrome and Long Qt Syndrome8, X-linked retinal disorder, Hyperaldosteronism, Spinocerebellar Ataxia 42, Primary Aldosteronism, Seizures, And Neurologic Abnormalities and Sinoatrial Node Dysfunction And Deafness, Neurodevelopmental Disorder, hypokalemic periodic paralysis, Epilepsy, developmental and epileptic encephalopathies, Brody myopathy, Darier's disease/Heart disease, von Willebrand disease, and Zellweger syndrome. In one embodiment, the disease or disorder is any caused by a genetic mutation that is amenable CRISPR-Cas9 mediated editing.

In one embodiment, the method of the present invention comprises administering to a subject having Duchenne Muscular Dystrophy a composition comprising a first nucleic acid comprising a coding region encoding a first portion of Dystrophin and a 3′ ribozyme, and a second nucleic acid comprising a coding region encoding a second portion of Dystrophin and a 5′ ribozyme, wherein the first nucleic acid transcribes a first RNA molecule and the second nucleic acid transcribes a second RNA molecule, and wherein cis-cleavage of the 3′ and 5′ ribozymes and trans-splicing of the coding region encoding the first portion of Dystrophin and the coding region encoding the second portion of Dystrophin, generates a single RNA molecule encoding a full-length Dystrophin protein.

In one embodiment, the method of the present invention comprises administering to a subject having Duchenne Muscular Dystrophy a composition comprising a first nucleic acid encoding the nucleic acid sequence of SEQ ID NO: 129 and a second nucleic acid encoding the nucleic acid sequence of SEQ ID NO: 130, wherein the first nucleic acid transcribes a first RNA molecule and the second nucleic acid transcribes a second RNA molecule, and wherein cis-cleavage of the 3′ and 5′ ribozymes and trans-splicing of the first RNA molecule and second RNA molecule, generates a single RNA molecule encoding a full-length Dystrophin protein.

In one embodiment, the method of the present invention comprises administering to a subject having Duchenne Muscular Dystrophy a composition comprising a first nucleic acid encoding the nucleic acid sequence of SEQ ID NO: 22 and a second nucleic acid encoding the nucleic acid sequence of SEQ ID NO: 23, wherein the first nucleic acid transcribes a first RNA molecule and the second nucleic acid transcribes a second RNA molecule, and wherein cis-cleavage of the 3′ and 5′ ribozymes and trans-splicing of the first RNA molecule and second RNA molecule, generates a single RNA molecule encoding a full-length Dystrophin protein with a C-terminal GFP reporter. In one embodiment, the second nucleic acid encodes a fragment of SEQ ID NO: 23, wherein the fragment does not include the coding sequence for the C-terminal GFP reporter.

In one embodiment, the method comprises administering to a subject having Duchenne Muscular Dystrophy a composition comprising a first RNA molecule encoding a first portion of Dystrophin and comprising a 3′ ribozyme, and a second RNA molecule encoding a second portion of Dystrophin and comprising a 5′ ribozyme, wherein cis-cleavage of the 3′ and 5′ ribozymes and trans-splicing of the first and second RNA molecules generates a single RNA molecule encoding a full-length Dystrophin protein.

In one embodiment, the method comprises administering to a subject having Duchenne Muscular Dystrophy a composition comprising a first RNA molecule comprising the nucleic acid sequence of SEQ ID NO: 129, and a second RNA molecule comprising the nucleic acid sequence of SEQ ID NO: 130, wherein cis-cleavage of the 3′ and 5′ ribozymes and trans-splicing of the first and second RNA molecules generates a single RNA molecule encoding a full-length Dystrophin protein.

In one embodiment, the method comprises administering to a subject having Duchenne Muscular Dystrophy a composition comprising a first RNA molecule comprising the nucleic acid sequence of SEQ ID NO: 22, and a second RNA molecule comprising the nucleic acid sequence of SEQ ID NO: 23, wherein cis-cleavage of the 3′ and 5′ ribozymes and trans-splicing of the first and second RNA molecules generates a single RNA molecule encoding a full-length Dystrophin protein with a C-terminal GFP reporter. In one embodiment, the second nucleic acid encodes a fragment of SEQ ID NO: 23, wherein the fragment does not include the coding sequence for the C-terminal GFP reporter.

In one embodiment, the method of the present invention comprises administering to a subject having one or more disease selected from Table 1 a composition comprising a first nucleic acid comprising a coding region encoding a first portion of a therapeutic protein corresponding to the related disease in Table 1 and a 3′ ribozyme, and a second nucleic acid comprising a coding region encoding a second portion of a therapeutic protein corresponding to the related disease in Table 1 and a 5′ ribozyme, wherein the first nucleic acid transcribes a first RNA molecule and the second nucleic acid transcribes a second RNA molecule, and wherein cis-cleavage of the 3′ and 5′ ribozymes and trans-splicing of the coding region encoding a first portion of the therapeutic protein and the coding region encoding the second portion of the therapeutic protein, generates a single RNA molecule encoding the full-length therapeutic protein

In one embodiment, the method comprises administering to a subject having one or more disease selected from Table 1 a composition comprising a first RNA molecule encoding a first portion of a therapeutic protein corresponding to the related disease in Table 1 and comprising a 3′ ribozyme, and a second RNA molecule encoding a second portion of a therapeutic protein corresponding to the related disease in Table 1 and comprising a 5′ ribozyme, wherein cis-cleavage of the 3′ and 5′ ribozymes and trans-splicing of the first and second RNA molecules generates a single RNA molecule encoding the full-length therapeutic protein.

TABLE 1
List of monogenic diseases caused by mutations in large genes, including
the protein size (# of amino acids), gene symbol, protein name and disease name.

| Protein | | | |
|---|---|---|---|
| Size | Gene | Therapeutic Protein | Disease |
| 3,685 | DMD | Dystrophin | Duchenne Muscular Dystrophy |
| 4,074 | PKHD1 | Fibrocystin | autosomal recessive polycystic |
| | | | kidney disease |
| 2,351 | F8 | Coagulation factor VIII | Hemophilia A |
| 2,273 | ABCA4 | Retinal-specific | Stargardt macular degeneration |
| | | phospholipid-transporting | |
| | | ATPase | |
| 2,080 | DYSF | Dysferlin | limb-girdle muscular |
| | | | dystrophies |
| 1,997 | OTOF | Otoferlin | DFNB9, neurosensory |
| | | | nonsyndromic recessive |
| | | | deafness |
| 1480 | CFTR | Cystic fibrosis | Cystic Fibrosis |
| | | transmembrane conductance | |
| | | regulator | |
| 1,465 | ATP7B | Copper-transporting | Wilson Disease |
| | | ATPase 2 | |
| 2,061 | MYOF | Myoferlin | Miyoshi Muscular Dystrophy |
| | | | and Deafness, Autosomal |
| | | | Recessive 9 |
| 2,215 | MY07A | Unconventional myosin- | Usher Syndrome, Type 1 and |
| | | Vila | Deafness, Autosomal Recessive 2 |
| 3,530 | MYO15A | Unconventional myosin-XV | Deafness, Autosomal Recessive |
| | | | 3 and Nonsyndromic Hearing Loss |
| 3,354 | CDH23 | Cadherin-23 | Usher syndrome type 1 |
| 1,809 | STRC | Stereocilin | autosomal recessive deafness- |
| | | | 16 (DFNB16) |
| 2,925 | OTOG | Otogelin | Meniere's disease (MD) |
| 2,155 | TECTA | Alpha-tectorin | Deafness, Autosomal |
| | | | Dominant 12 and Deafness, |
| | | | Autosomal Recessive 21 |
| 1,955 | PCDH15 | Protocadherin-15 | Usher syndrome Type IF |
| | | | (USHIF) and DFNB23 |
| 2,365 | TRIOBP | TRIO and F-actin-binding | Deafness, Autosomal |
| | | protein | Recessive 28 and |
| | | | Nonsyndromic Hearing Loss |
| 1,616 | MY03A | Myosin-IIIa | Deafness, Autosomal Recessive |
| | | | 30 and Nonsyndromic Hearing Loss |
| 1,736 | COL11A2 | Collagen alpha-2(XI) chain | Otospondylomegaepiphyseal |
| | | | Dysplasia, Autosomal |
| | | | Recessive and |
| | | | Otospondylomegaepiphyseal |
| | | | Dysplasia, Autosomal |
| | | | Dominant |
| 2,067 | LOXHD1 | Lipoxygenase homology | Deafness, Autosomal Recessive |
| | | domain-containing protein 1 | 77 and Autosomal Recessive |
| | | | Non-Syndromic Sensorineural |
| | | | Deafness Type Dfnb |
| 2,332 | PTPRQ | Phosphatidylinositol | autosomal-recessive |
| | | phosphatase PTPRQ | nonsyndromic hearing |
| | | | impairment DFNB 84 |
| 2,332 | OTOGL | Otogelin-like protein | Deafness, Autosomal Recessive |
| | | | 84B and Rare Genetic Deafness |
| 1,995 | MYH14 | Myosin-14 | Peripheral Neuropathy, |
| | | | Myopathy, Hoarseness, And |
| | | | Hearing Loss and Deafness, |
| | | | Autosomal Dominant 4A |
| 1,960 | MYH9 | Myosin-9 | congenital thrombocytopenia, |
| | | | sensory hearing loss |
| 2,201 | TNC | Tenascin | DFNA56, HXB, deafness, |
| | | | autosomal dominant 56, |
| | | | hexabrachion |
| 2,506 | CACNA1A | Voltage-dependent P/Q-type | epileptic encephalopathy |
| | | calcium channel subunit | |
| | | alpha-1A | |
| 2,221 | CACNA1C | Voltage-dependent L-type | Timothy Syndrome and Long |
| | | calcium channel subunit | Qt Syndrome8 |
| | | alpha-1C | |
| 1,977 | CACNAIF | Voltage-dependent L-type | X-linked retinal disorder |
| | | calcium channel subunit | |
| | | alpha-1F | |
| 2,353 | CACNAIH | Voltage-dependent T-type | Hyperaldosteronism |
| | | calcium channel subunit | |
| | | alpha-1H | |
| 2,377 | CACNA1G | Voltage-dependent T-type | Spinocerebellar Ataxia 42 |
| | | calcium channel subunit | |
| | | alpha-1G | |
| 2,161 | CACNAID | Voltage-dependent L-type | Primary Aldosteronism, |
| | | calcium channel subunit | Seizures, And Neurologic |
| | | alpha-1D | Abnormalities and Sinoatrial |
| | | | Node Dysfunction And |
| | | | Deafness |
| 2,339 | CACNA1B | Voltage-dependent N-type | Neurodevelopmental Disorder |
| | | calcium channel subunit | |
| | | alpha-1B | |
| 1,873 | CACNAIS | Voltage-dependent L-type | hypokalemic periodic paralysis |
| | | calcium channel subunit | |
| | | alpha-1S | |
| 2,223 | CACNA1I | Voltage-dependent T-type | Epilepsy |
| | | calcium channel subunit | |
| | | alpha-1I | |
| 2,313 | CACNA1E | Voltage-dependent R-type | developmental and epileptic |
| | | calcium channel subunit | encephalopathies |
| | | alpha-1E | |
| 1,001 | ATP2A1 | Sarcoplasmic/endoplasmic | Brody myopathy |
| | | reticulum calcium ATPase 1 | |
| 1,042 | ATP2A2 | Sarcoplasmic/endoplasmic | Darier's disease/ Heart disease |
| 2,813 | VWF | reticulum calcium ATPase 2 | von Willebrand disease |
| | | von Willebrand factor | |
| 1,283 | PEX1 | Peroxisome biogenesis factor 1 | Zellweger syndrome |
| 4,069 | CMYA5 | Cardiomyopathy-associated | |
| | | protein 5Cardiomyopathy | |

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Example 1: Ribozyme-Mediated RNA Assembly and Expression in Mammalian Cells

Ribozymes (Rzs) are small catalytic RNA sequences which are capable of nucleotide-specific self-cleavage (Doherty and Doudna 2000). Ribozyme-mediated RNA cleavage generates unique 3′ phosphate and 5′-hydroxy termini, which resemble substrates for ubiquitous RNA repair pathways present in all three kingdoms of life. As shown herein, ribozyme-mediated cis-cleavage can be harnessed for the trans-splicing of independent RNA transcripts in mammalian cells, an approach named stitchR (stitch RNA). Remarkably, reconstitution of messenger RNA by stitchR allowed for efficient translation and expression of full-length proteins in mammalian cells. As demonstrated, stitchR can be harnessed for the combination of protein coding functional domains or for the delivery and expression of large protein coding sequences by viral vectors. Further, overexpression of RNA 2′,3′-Cyclic Phosphate and 5′-OH (RtcB) Ligase enhances stitchR activity in mammalian cells and is sufficient for catalyzing stitchR activity in vitro. These data characterize a novel approach utilizing ribozymes for the scar-less trans-splicing of functional RNAs in cells which could be useful for myriad research and therapeutic applications.

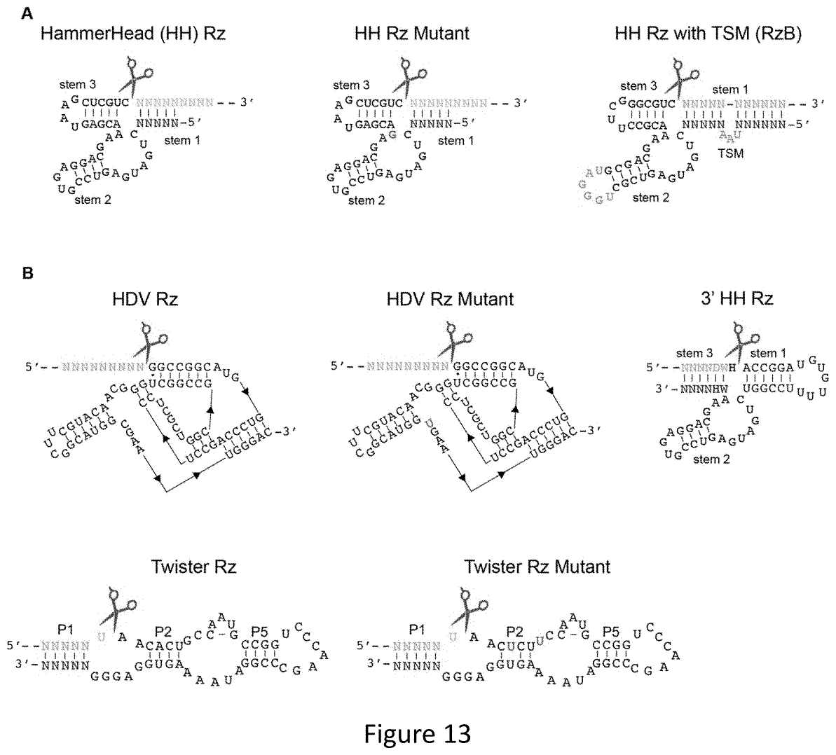

Autocatalytic RNA sequences are widespread in nature and catalyze diverse biological processes, including intron splicing, rolling circle viral genome replication, and peptide bond formation (Weinberg et al. 2019). At least seven major ribozyme families have been identified with distinct sequence and structural features, including Hammerhead (HH), Hepatitis Delta Virus (HDV), Varkud Satellite (VS), Sister, Twister-sister, Hairpin, Hatchet and Pistol. Most widely studied are the HH, HDV, and Twister family members, which due to their small size and cleavage characteristics, have been utilized in vitro and in vivo to generate RNAs with precise termini devoid of ribozyme sequences (FIG. 13) (Ferre-D′Amare and Doudna 1996; Avis et al. 2012; Zhang et al. 2017).

In prokaryotes and eukaryotes, most cellular RNAs are synthesized and spliced with 5′-phosphate (P) and 3′-hydroxyl (OH) termini, including messenger and long noncoding RNA. In contrast, unconventional cis-splicing of many tRNAs and the mRNA encoding the ER stress-responsive protein XBP1, are catalyzed by enzymatic pathways which result in unique 5′-OH and either 3′-P or 2′3′ cyclic Phosphate (cP) ends. Recent findings suggest that unconventional cis-splicing of RNA is catalyzed by the ubiquitous RNA 2′,3′-Cyclic Phosphate and 5′-OH (RtcB) ligase in mammals. Additionally, RtcB and several other enzyme families may function to repair host cell RNAs which have been damaged by stress or exogenous ribotoxins. Since ribozyme-mediated cleavage results in similar terminal ends, ribozyme-cleaved RNAs could be subject to trans-splicing by endogenous RNA repair pathways.

Ribozyme-Cleaved mRNAs are Trans-Spliced and Translated in Mammalian Cells

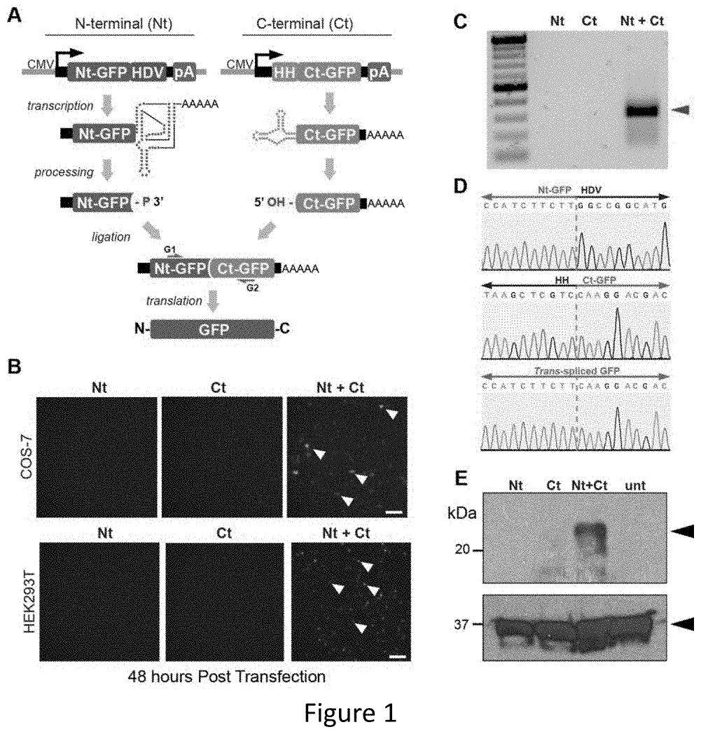

To determine whether ribozymes could be utilized for scar-less trans-splicing of RNA in mammalian cells, two expression plasmids were designed containing non-overlapping N-terminal (Nt) and C-terminal (Ct) fragments of the fluorescent reporter GFP (Nt-GFP and Ct-GFP, respectively). Ribozymes were designed to catalyze their own removal from adjacent nucleotides of the GFP fragments, including a 3′ HDV ribozyme on Nt-GFP and a 5′ HH ribozyme on Ct-GFP (FIG. 1A). Expression of either the Nt or Ct encoding GFP-ribozyme RNAs alone resulted in no detectable GFP fluorescence when transfected into mammalian COS-7 or HEK293T cells (FIG. 1B). Remarkably, co-expression of the Nt- and Ct-GFP encoded RNAs together resulted in green fluoresce after 48 hours (FIG. 1B). RT-PCR analysis and sanger sequencing revealed that trans-splicing of the separate Nt- and Ct-GFP RNAs had occurred between the predicted ribozyme-catalyzed cleavage sites (FIG. 1C and FIG. 1D). Further, full-length GFP protein was detected by western blot in co-transfected cells (FIG. 1E). These data demonstrate that endogenous mammalian cellular RNA repair pathways were sufficient to catalyze the trans-splicing of independent ribozyme-processed RNAs, which were efficiently translated into full-length protein. This RNA trans-splicing approach was named stitchR.

### Impact of Ribozyme Sequence and Type on Ribozyme-mediated Trans-Splicing

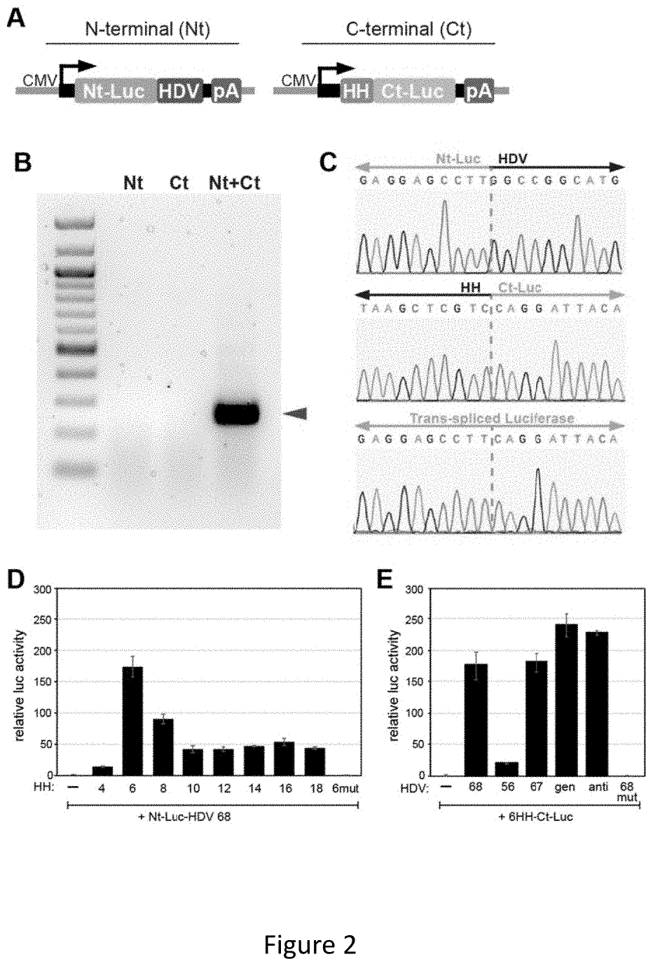

To precisely quantify the relative amount of functional, full-length protein generated by ribozyme-mediated trans splicing in cells, a reporter was generated using two non-overlapping halves of firefly Luciferase (FIG. 2A). Consistent with our previous findings, only co-transfection of both Nt- and Ct-Luciferase-ribozyme encoding RNAs resulted in trans-splicing and luciferase activity in cells (FIG. 2B and FIG. 2C). Using this assay, the effects of different HH and HDV ribozyme sequences on trans-splicing activity in mammalian cells was further characterized. A 6 base-pair (bp) overlap in Stem 1 HH ribozyme provided the greatest luciferase activity and mutation of a HH catalytic residue abolished activity, consistent with previous reports for HH ribozyme activity characterized in vitro (FIG. 2D). Additionally, both genomic and antigenomic HDV ribozyme sequences were comparable in luciferase activity, with the exception of the minimal 56 nucleotide HDV ribozyme (HDV56), which showed significantly reduced activity (FIG. 2E). Also consistent with previous reports, a C to U mutation of a nucleotide required for HDV catalysis resulted in a complete loss of luciferase activity (FIG. 2E). These findings demonstrate that ribozyme-mediated trans splicing activity is dependent upon ribozyme cleavage in mammalian cells.

Prevention of Unwanted or Truncated Protein Expression from Nt or Ct Vectors using Translational Control and/or Protein Degradation Sequences

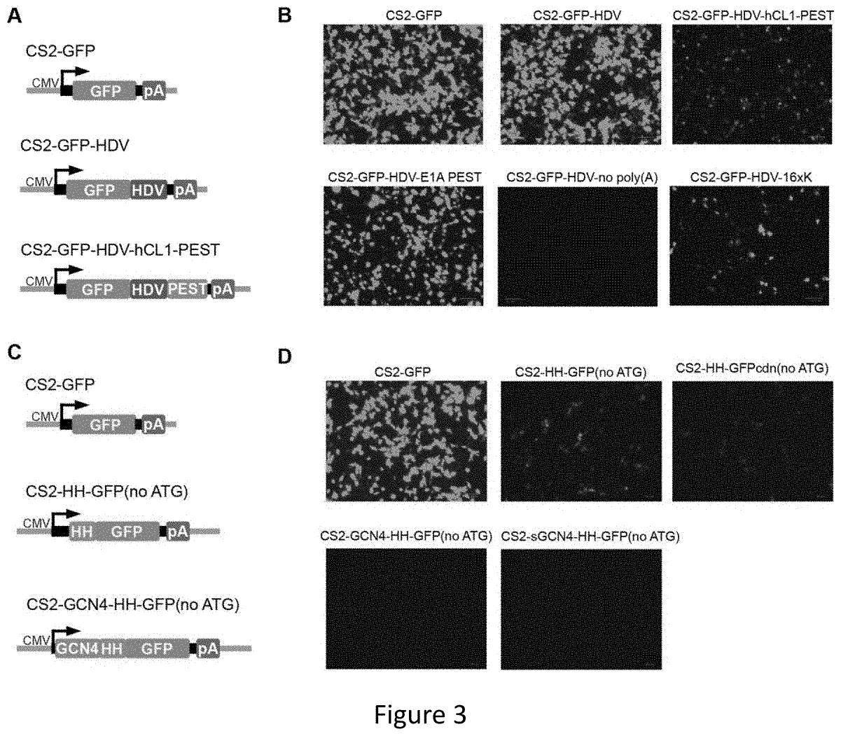

Nt or Ct RNAs could be subject to translation prior to ribozyme-mediated cleavage, or when expressed separately, potentially resulting in unwanted or truncated protein expression. To limit the expression of un-spliced Nt or Ct vectors, the efficacy of previously characterized translational control of protein degradation sequence on the stability of vectors encoding full-length GFP was tested. Addition of an HDV ribozyme on the 3′ end of GFP did not appear to alter GFP fluorescence (FIGS. 3A and B). To selectively prevent the expression of GFP, the effect of protein degradation sequences hCL1-PEST, E1A-PEST, removal of the vector's poly(A) sequence or simulated translation through a poly A tail to generate a poly K tail was tested (FIGS. 3A and B). All degradation sequences were cloned in-frame with the GFP open reading frame, such that translation occurred through the HDV ribozyme sequence. Inclusion of an hCL1-PEST showed a strong reduction in GFP fluorescence, whereas the EF1a PEST did not. Deletion of the vector poly (A) sequence from the expression vector resulted in complete loss of GFP expression, and translation through a poly A sequence to generate a poly K tail also resulted in decreased fluorescence.

For a Ct encoded GFP reporter, inclusion of a 5′ HH ribozyme and deletion of the GFP start codon (ATG) still resulted in weak, but detectable GFP expression, despite a lack of predicted upstream alternative ATGs (FIGS. 3C and D). Further silent mutations within N-terminal NTG codons in GFP (GFPcdn) further decreased GFP detection, however, weak fluorescence was still evident. Inclusion of the 5′ UTR of yeast GCN4 gene, which encodes 4 small upstream ORFs which function as translational inhibitors, abolished detectable GFP fluorescence. A smaller internal fragment of the GCN4 5′ UTR encoding only the 4 uORFs was similarly effective at preventing GFP expression. These data demonstrate that translational control of protein degradation sequences can be utilized to prevent unwanted protein expression from individual Nt or Ct vectors.

These translational control or protein degradation sequences could be utilized for other dual vector applications where limiting unwanted or truncated protein expression is desired, such as dual AAV vector strategies which rely on homologous recombination to generate large protein coding open reading frames.

### Single and Multi-Plex Trans-Splicing of Function Protein Coding RNAs

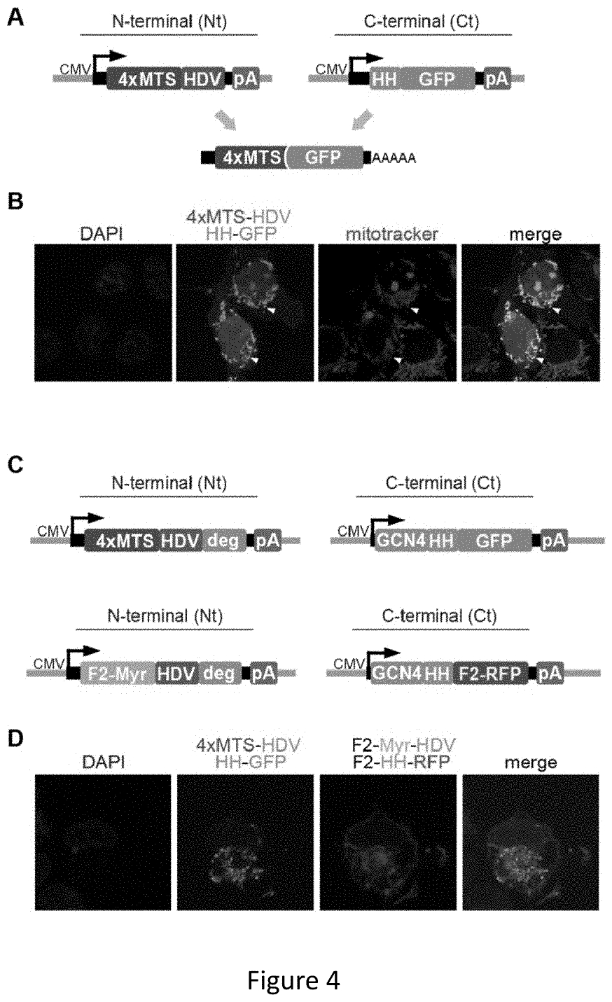

To determine whether ribozyme-mediated trans-splicing could be used for the combination of protein coding functional domains in cells, RNAs encoding 4 copies of a mitochondrial targeting sequence (Nt-4xMTS) and an open reading frame encoding full-length GFP, lacking its ATG start codon (Ct-GFP), were generated (FIG. 4A). Co-expression of these two independent RNAs resulted in robust expression of mitochondrial-localized GFP, which overlapped with the red fluorescent mitochondrial marker MitoTracker Red CMXRos (FIG. 4B). These findings demonstrate that ribozyme-mediated trans-splicing can be used to rapidly combine two independent RNAs to express specific functional fusion proteins in cells.

Ribozyme mediated trans-splicing and expression of multiple different functional proteins at the same time may also be possible due to the three open reading frames in which proteins are translated. By harnessing this feature, functional proteins can be generated using trans-splicing of RNAs which are in compatible three different open reading frames. To demonstrate this functionality, an additional ribozyme pair in reading frame 2 (F2) which encoded a myristoylation membrane targeting sequence (Nt-F2-Myr) and red fluorescent protein (Ct-F2-RFP) were designed (FIG. 4C). These Nt and Ct vector pairs also included the hCL1-PEST protein degradation sequence and GCN4 translational inhibitory sequences to limit truncated protein expression from individual Nt and Ct vectors, respectively. In co-transfected cells, GFP fluorescence was highly specific to mitochondria and RFP fluorescence was highly specific to membranes (FIG. 4D), demonstrating the ability of this approach for trans-splicing of RNA to generate different functional proteins in cells.

### Optimized Ribozymes Enhance Protein Expression in Ribozyme-Mediated Trans-Splicing

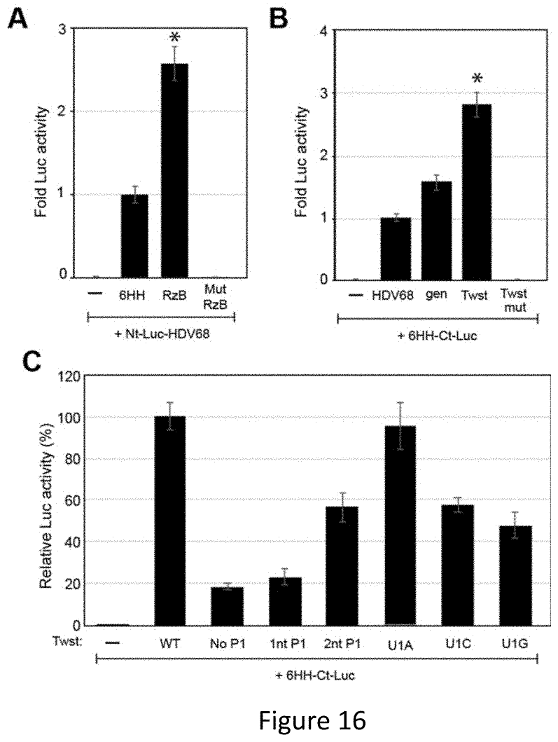

Small sequence modifications can profoundly impact ribozyme catalytic activity by altering secondary structure, stability or binding to metal ion cofactors. Using our trans-splicing luciferase reporter assay, we identified improved ribozyme types and sequence modifications which enhance trans-splicing luciferase reporter activity in mammalian cells (FIG. 16). The RzB Hammerhead variant ribozyme, which contains a tertiary stabilizing motif (TSM), showed greater activity than a ribozyme without a TSM (FIG. 16A). Further, a Twister (twst) ribozyme showed greater activity than HDV ribozymes, when cloned 3′ to Nt-Luc. Catalytic mutations within the twister ribozyme could similarly abolish luciferase activity (FIG. 16B) and are dependent upon P1 stem formation (FIG. 16C). Since Twister ribozymes require a U at position 1, this requirement could limit the design of scar-less trans-splicing to sequences which end in U. Therefore, we tested whether nucleotide substitutions could be tolerated at position 1, and found a U1A was not show significantly different activity, while U1C or U1G substitutions retained activity, albeit somewhat reduced (FIG. 16C).

### Optimized Splice Donor and Acceptor Sequences Enhance Protein Expression in Ribozyme-Mediated Trans-Splicing

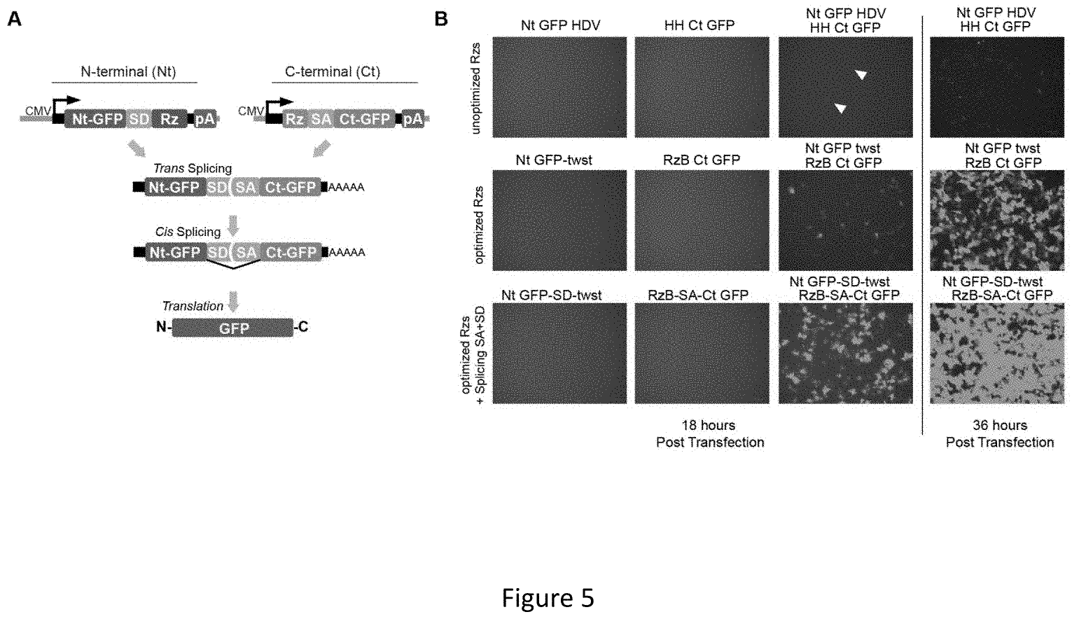

Pre-mRNA splicing by the spliceosome has been shown to enhance mRNA translation, either through deposition of factors which promote a pioneer round of translation or through promoting RNA processing and export to the cytoplasm. The addition of chimeric cis-splicing intron within a transgene has also been shown to promote transgene protein expression. It was then investigated whether trans-spliced RNAs could undergo cis-splicing by the spliceosome, and whether this would impact translation and expression of trans-spliced mRNAs. To test this, Splice Donor (SD) and Splice Acceptor (SA) sequences were incorporated within the trans-splicing GFP reporter, such that the trans-spliced RNA would reconstitute a chimeric intron (FIG. 5A). Remarkably, the addition of SD and SA sequences resulted in a robust enhancement of GFP fluorescence, compared to trans-splicing GFP reporters without SD or SA sequences (FIG. 5B). RT-PCR and sanger sequencing showed that Nt-GFP and Ct-GFP RNAs containing SD and SA sequences were both trans- and cis-spliced, resulting in restoration of the normal GFP open reading frame (data not shown). These data suggest that trans-splicing may occur in the nucleus, and that subsequent cis-splicing is a useful strategy for enhancing the expression from trans-spliced RNAs.

Ribozyme-Mediated Trans-Splicing and Expression of Large Gene Sequences for Delivery using Viral Therapeutic Vectors

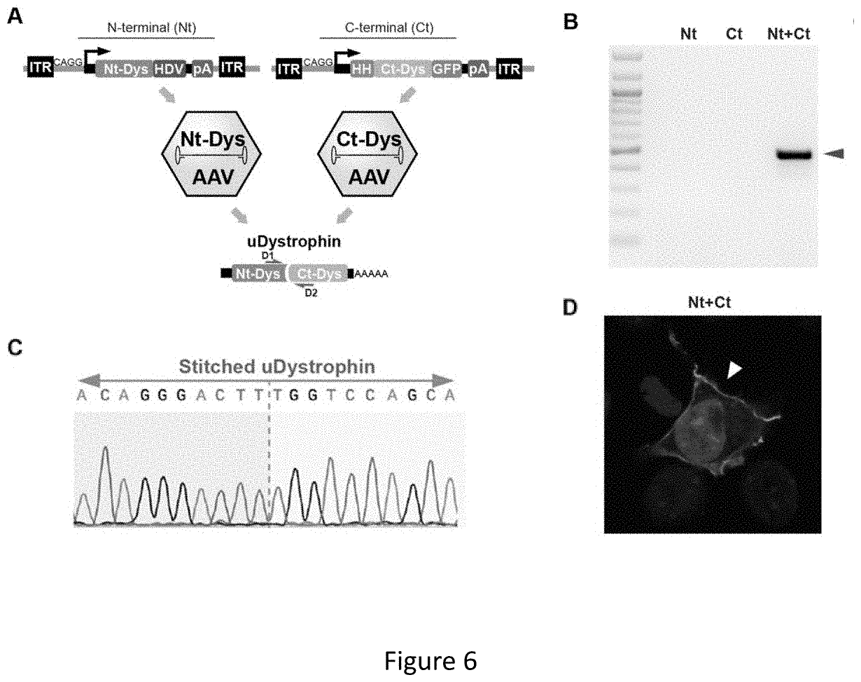

Ribozyme-mediated trans-splicing could be harnessed for the delivery and expression of large protein coding mRNAs which exceed the packaging size limit for therapeutic viral gene therapy vectors, such as AAV (FIG. 6A). This could be useful to restore expression of large genes mutated in numerous human monogenic diseases, such as Dystrophin (Dys) in Duchenne Muscular Dystrophies (DMDs), CFTR in Cystic Fibrosis (CF), Factor VIII (F8) in Hemophilia A, etc. In cell-based transfection assays, co-expression of vectors encoding Nt and Ct- split μDystrophin with C-terminal GFP tag were trans-spliced in mammalian cells (FIG. 6B and FIG. 6C) and localized to membranes (FIG. 6D). These data demonstrate the feasibility of using ribozyme-mediated trans-splicing to reconstitute and express large protein coding genes.

### Lentiviral Delivery of Ribozyme-Enabled RNAs for Trans-Splicing in Cells

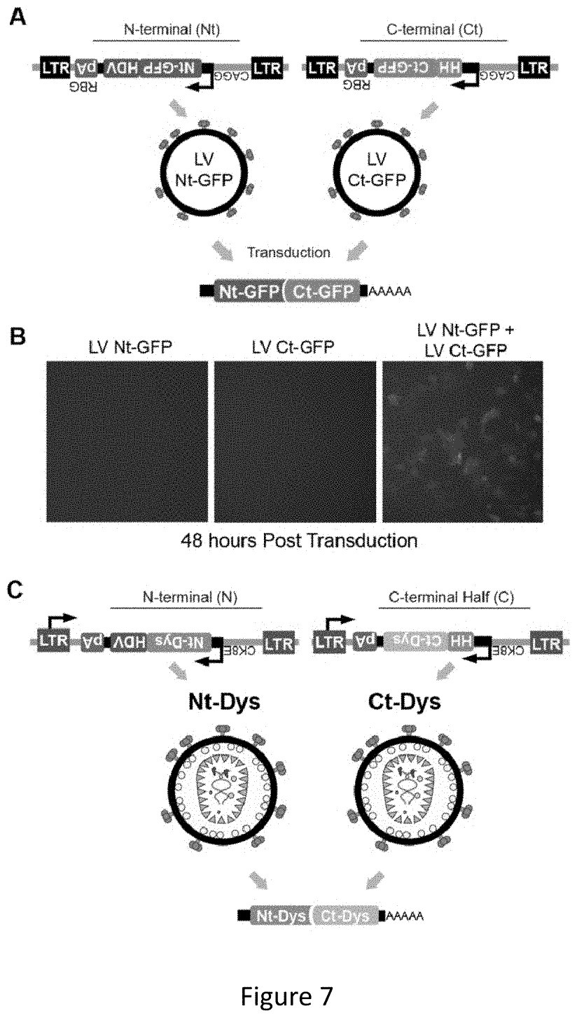

The autocatalytic self-cleavage of ribozymes could hinder the packaging of ribozyme-encoding RNAs by positive-sense RNA viruses, such as commonly used gamma retrovirus and lentivirus vectors. To circumvent this potential issue, Nt and Ct split GFP expression cassettes were encoded on the negative sense strand in 3rd generation lentiviral vector backbones (FIG. 7A). Lentiviral particles were generated separately for Nt and Ct vectors, which were then used to transduce HEK293T cells. Cells transduced with both Nt-GFP and Ct-GFP showed green fluorescence expression, while cells transduced with either Nt-GFP or Ct-GFP alone showed no detectable fluorescence (FIG. 7B). These data demonstrate that lentiviral vectors are capable of delivery and expression of ribozymes encoding RNAs for trans-splicing.

This approach could be also useful for delivery of large gene sequences which exceed the packaging size of these viral vectors, such as Dys (FIG. 7C). Ribozyme-mediated trans splicing could also allow for the safe handling or reconstitution of viral genomes, such as lentivirus or large coronavirus RNA genomes.

Safe Handling, Delivery and Expression of Toxic Genes or Antiviral Gene using Viral Vectors

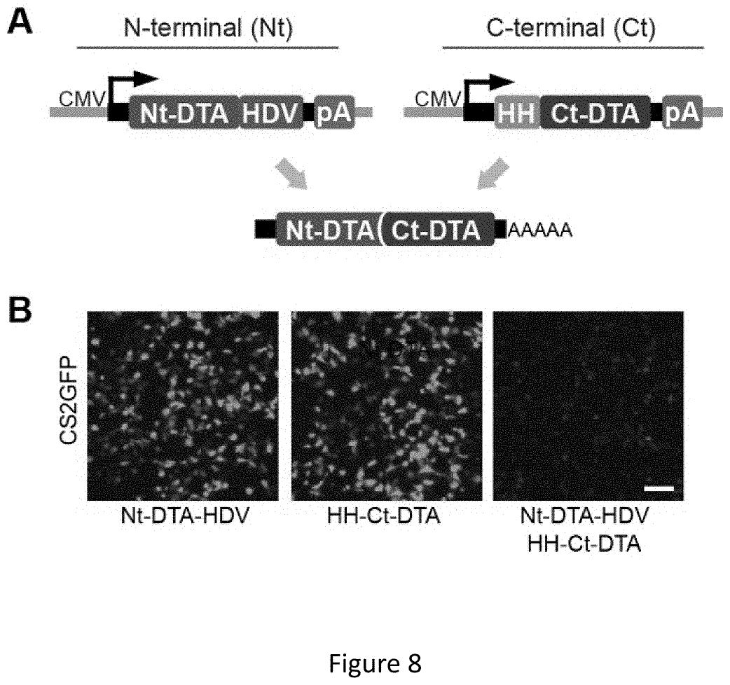

Ribozyme-mediated trans splicing could also allow for the safe handling or reconstitution of toxic or antiviral proteins which may inhibit generation of lentiviral particles in mammalian packaging cells. These include a number of cell suicide genes, such as the translational inhibitory diptheria toxin A (DTA) (FIG. 8A). We show that vectors encoding a split DTA sequence, upon trans-splicing and expression, inhibit the co-expression of a CS2GFP reporter construct, consistent with the translational inhibitory role of DTA in mammalian cells (FIG. 8B).

### Enzymes to Enhance or Inhibit Ribozyme-Mediated Trans-Splicing

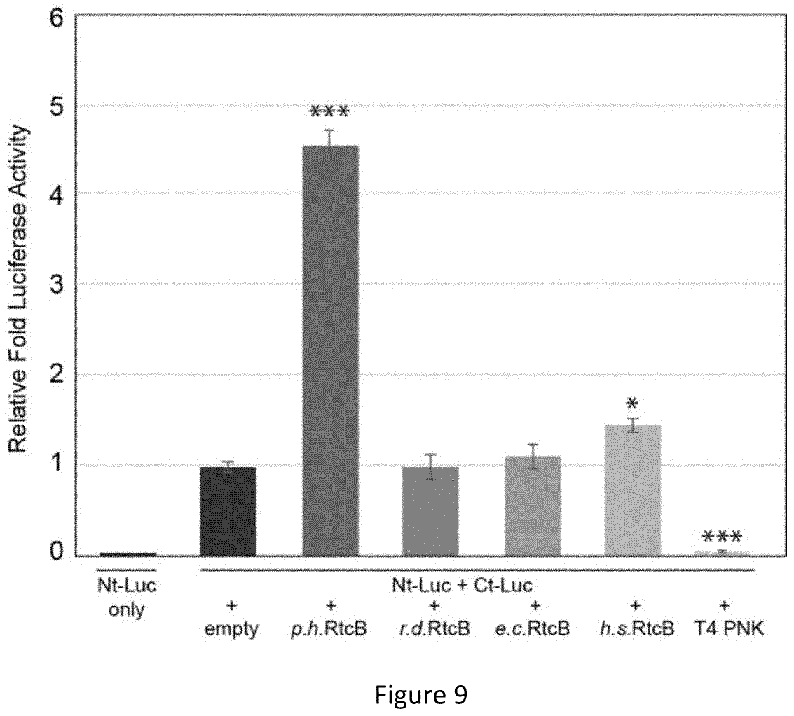

A number of enzyme families have been suggested to ligate 5′-OH and either 3′-P or 2′3′ cyclic Phosphate (cP) ends, most notably RtcB which is found conserved in all three domains of life. Human codon optimized RtcB orthologs from Eukarya (H. sapiens), Bacteria (E. coli) and Archaea (P. horikoshii) species were cloned and co-expressed to measure their effects on the activity of the trans-splicing luciferase reporter. Interestingly, co-expression of RtcB from P. horikoshii resulted in enhanced (4.5-fold) activation of luciferase activity, while human and bacterial orthologs showed modest or no enhancement, respectively (FIG. 9).

Other enzyme families have been shown to modulate these RNA termini. Interestingly, expression of T4 polynucleotide kinase (T4 PNK), which acts as a 5′-hydroxyl kinase and 3′-phosphatase and a 2′,3′-cyclic phosphodiesterase, significantly inhibited luciferase activity (FIG. 9). These data show that co-expression of exogenous enzymes can both enhance or inhibit ribozyme-mediated trans-splicing in mammalian cells.

### RtcB is Sufficient to Catalyze Ribozyme-Mediated RNA Trans-Splicing In Vitro

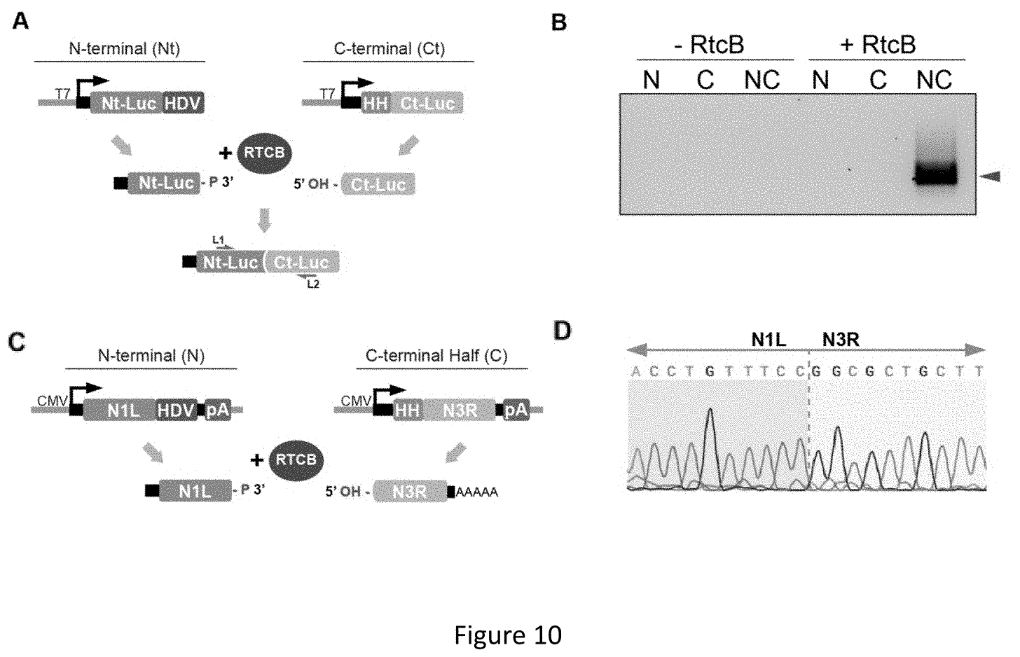

Due to their nucleotide-specific cleavage, ribozymes have been utilized extensively in vitro to generate precise RNA ends. It was next sought to determine if ribozymes could be used for directional trans-splicing of independently synthesized RNAs in vitro. Using in vitro RNA transcription of the Nt- and Ct-Luciferase-ribozyme reporter constructs using T7 RNA polymerase, it was found that the addition of recombinant E. coli RtcB was both necessary and sufficient to catalyze the trans-splicing, detected using RT-PCR (FIG. 10A and FIG. 10B). Similarly, RNAs encoding domains of the spider protein Spidroin were designed (FIG. 10C). Spidroin is the major component of spider dragline silk, a material revered for its tensile properties, but which has been difficult to synthesize in heterologous systems due to the highly repetitive nature of the protein. Spidroin naturally consists of multiple A and Q repeats, flanked by conserved N-terminal (N1L) and C-terminal (N3R) domains. Following in vitro synthesis of Spidroin RNAs with T7 polymerase, it was found that the addition of recombinant RtcB ligase from E. coli was sufficient to catalyze the trans-ligation of the ribozyme cleaved N1L and N3R encoding RNAs, as detected by RT-PCR and sanger sequencing (FIG. 10D).

### Controlled Tandem Trans-Splicing of RNA Encoding Multi-Domain Proteins

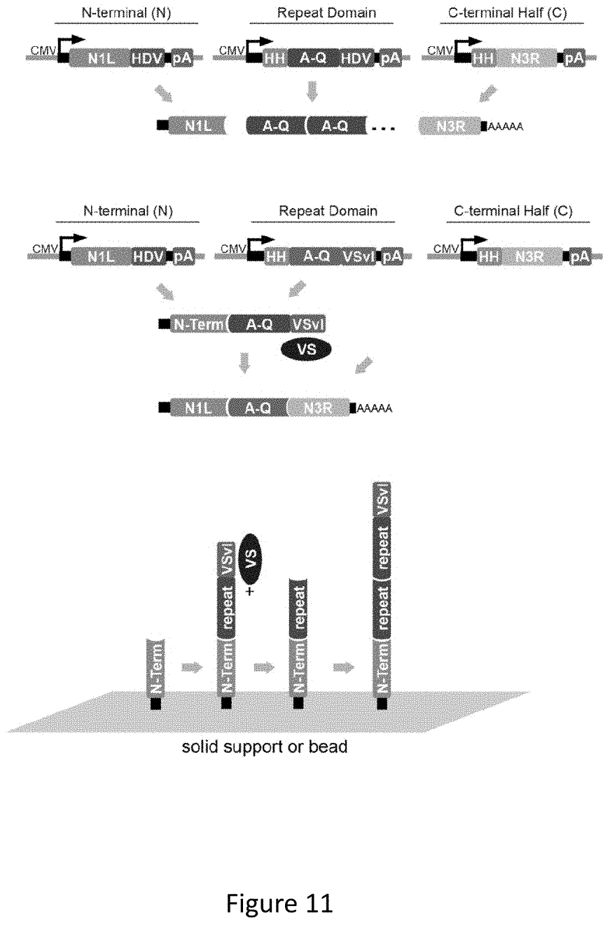

It was next examined whether the addition of a third RNA, encoding an A-Q fusion domain with flanking ribozymes, would result in tandem repeat assembly, albeit uncontrolled (FIG. 11A). While directional trans-splicing between each of the separate RNAs was able to be detected, the assembly of three or more independent RNA fragments was unable to be detected (data not shown). This may be due to the rapid circularization of RNAs which contain termini which are both compatible for ligation by RtcB. As an alternative approach, utilization of a trans-activated VS ribozyme has the potential to allow for the sequential and controlled assembly of RNAs sequences in vitro (FIG. 11B and FIG. 11C). In this approach, the 3′ terminal RNA ribozyme is only suitable for ligation by RtcB upon the addition and trans-cleavage by VS-Rz. Since the VS-Rz trans-activating ribozyme RNA is not covalently attached, stepwise addition of stitchR compatible RNAs, VS-Rz and RtcB ligase could allow for the controlled tandem assembly of RNA sequences, which may be useful for the assembly of repeat RNAs encoding biologically or industrially important proteins, such as synthetic spider silks, elastins, collagens, etc.

Trans-Splicing of Endogenous RNAs using Trans-Cleaving Ribozymes—Therapeutic Applications to Correct Disease Causing Mutations

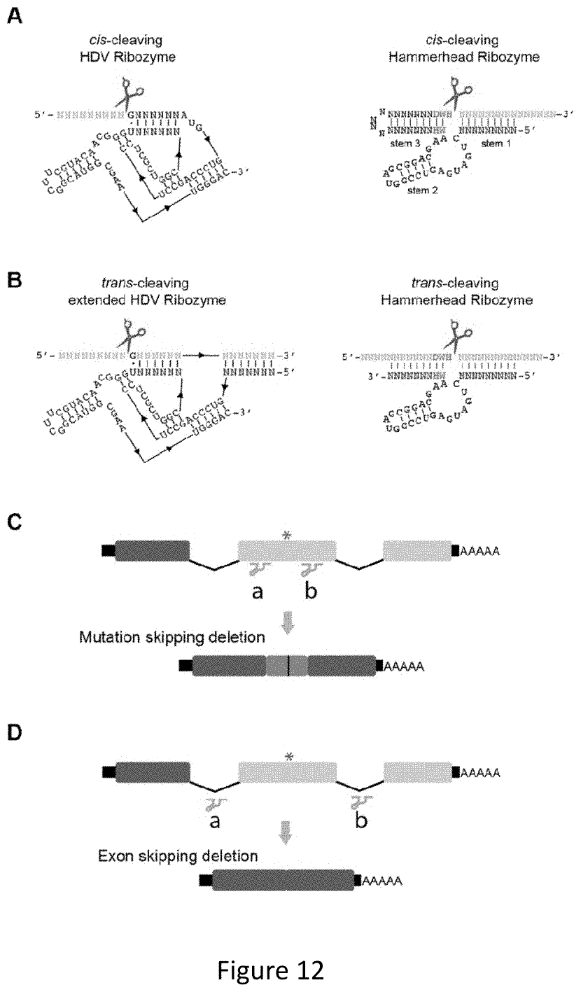

Ribozymes are autocatalytic RNAs which cleave in cis, to produce unique RNA termini that we have shown are trans-spliced and subsequently expressed in mammalian cells (FIG. 12A). Remarkably, cis-cleaving ribozymes can be engineered to cleave in trans, such that target RNAs can be cleaved in a nucleotide specific manner, resulting in similar RNA termini (FIG. 12B) (Carbonell et al. 2011; Webb and Luptak 2018). Thus, trans-cleaving ribozymes could be utilized to catalyze scarless trans-splicing of RNA in cells or in vitro. This approach could be useful for myriad applications, one major one being the deletion of disease causing mutations in gene transcripts by targeting mutation flanking sequences in either exon or intron sequences (FIG. 12C and FIG. 12D).

In conclusion, it is shown herein that ribozyme-mediated cleavage of independent RNAs expressed in cells are efficiently assembled and capable of translation in mammalian cells. This approach, which is termed stitchR herein, has the ability to function as a novel method for the combinatorial assembly of functional RNA and proteins for both basic and therapeutic applications. Due to the autocatalytic nature of ribozymes and the endogenous RNA repair pathways present in cells, stitchR only requires the expression of separate RNAs for trans-splicing and translation to occur in cells. In vitro, it is demonstrated that the RtcB ligase was sufficient for trans-splicing, and due to the ubiquitous and widespread expression of RtcB across all three kingdoms of life, stitchR has the potential to be a useful approach in many diverse organisms.

The robust nature of this system relies on the efficient and precise nature of ribozyme-mediated RNA cleavage, which produces reliable and precise nucleotide specific ends essential for the restoration of protein coding open reading frames. Further, the ability to generate RNAs using ribozymes which completely catalyze their own removal allows for scar-less assembly, resulting in RNAs which are essentially indistinguishable from their natural counterparts.

While ribozyme cleavage has been extensively studied in vitro, ribozyme cleavage in vivo is less well understood, and thought to be influenced by folding through interaction with RNA binding proteins and the availability of metal ions required for catalysis. StitchR serves as an indirect readout of ribozyme mediated cleavage, which interestingly was found herein to significantly influenced by changes in ribozyme sequence and structure. This suggests that optimization of ribozyme cleavage may be a useful approach for enhancing stitchR activity in vivo. Further analysis of the effects of RNA repair pathway components, such as RtcB, RtcA, and Archease, may also serve as important factors in regulating stitchR activity.

Ribozymes have naturally evolved to function in cis to promote their self-cleavage, however, a number of ribozyme families (notably HDV and HH) have been engineered to cleave target RNAs in trans. It is noted herien that combining trans-cleaving ribozymes with stitchR may further allow for a powerful RNA cleavage and repair method in cells or in vitro. This approach could serve as a nucleotide-specific ‘cut and paste’ approach for RNA which may be useful for generating RNA diversity or for removing certain deleterious mutations in disease causing RNAs.

### Example 2: Inducible Trans-Splicing and Expression of RNA using Trans-Activated Ribozymes

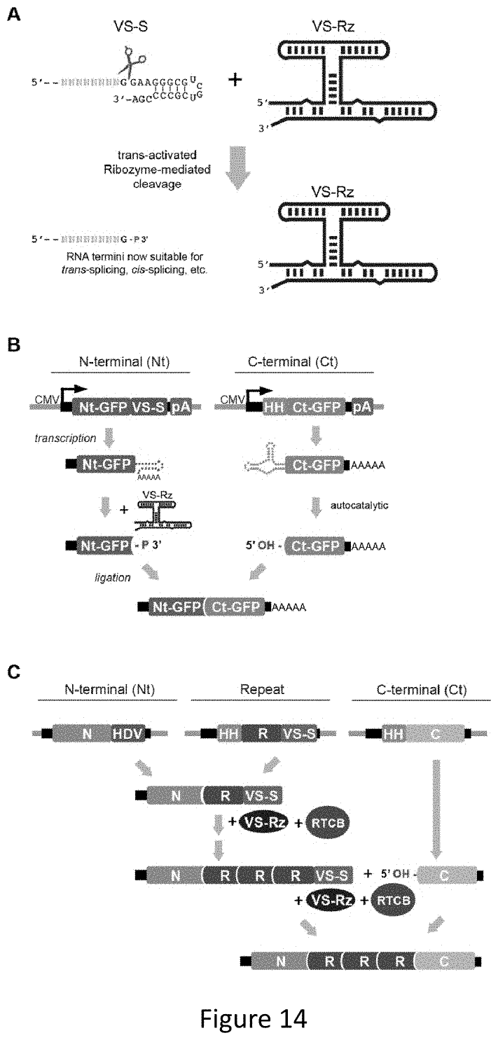

Most ribozymes are autocatalytic and only require metal ions as cofactors, readily found in biological environments, which aid in folding and chemical catalysis. The Varkud Satellite (VS) ribozyme can be utilized for scar-less trans-splicing, if the donor RNA ends in a G nucleotide. Interestingly, the VS ribozyme can be modified to allow for trans-activation of the ribozyme to induce catalysis (Guo and Collins 1995; Ouellet et al. 2009). When split into two components, the small VS stem loop (VS-S) is not alone sufficient to induce cis-cleavage, however, the addition of the remaining sequence, VS-Rz, promote efficient cleavage of the VS-S (FIG. 14A). This trans-activation feature could allow for inducible ribozyme-mediated trans-cleavage, where addition of VS-Rz sequence is required for VS-S cleavage on an Nt donor RNA, which could then be suitable for trans-splicing with an Ct acceptor RNA containing a 5′-OH termini (FIG. 14B). The VS-Rz sequence, which contains typical 5′-P- and 3′-OH RNA termini, cannot participate in trans-splicing, and thus may function as a multi-turnover catalyst of the reaction.

The ability to control ribozyme-mediated cleavage, such as through the required addition of a trans-activating sequence, such as VS-Rz, may allow for the controlled addition of variable or non-variable RNA sequences to generate synthetic repeat RNAs (FIG. 14C). One approach is to generate an RNA with a unique N-terminal domain, a unique C-terminal domain, and an internal variable or non-variable ‘repeat’ domain. This approach would require both the N-terminal and C-terminal RNAs to contain a single ribozyme on the 3′ and 5′ ends, respectively. The internal repeat RNA would require ribozymes on both 5′ and 3′ ends, to allow it to function as both an acceptor and donor during trans-splicing. However, the addition of ribozymes on both termini of an RNA, or an RNA with both 3′-P and 5′-OH, leads to circularization by ligases, such as RtcB (Desai et al. 2015), preventing participation in a growing linear chain. However, the utilization of an inducible trans-activated ribozyme could allow for step-wise ligation of 5′ and 3′ ends through addition and removal of both VS-Rz and RtcB ligase, leading to controlled RNA domain synthesis (FIG. 14C). This approach could be useful for generation of highly repetitive RNA sequences, which could be subsequently translated to create synthetic repeat proteins, such as those composing hydrogels, synthetic spider silks, or collagens, etc, which can be difficult to generate and encode as DNA due to recombination. These approaches may be useful for drug delivery, generation of biomaterials or industrial materials (Chambre et al. 2020).

### Example 3: Generation of Stable Synthetic Intronic Sequences using Ribozymes

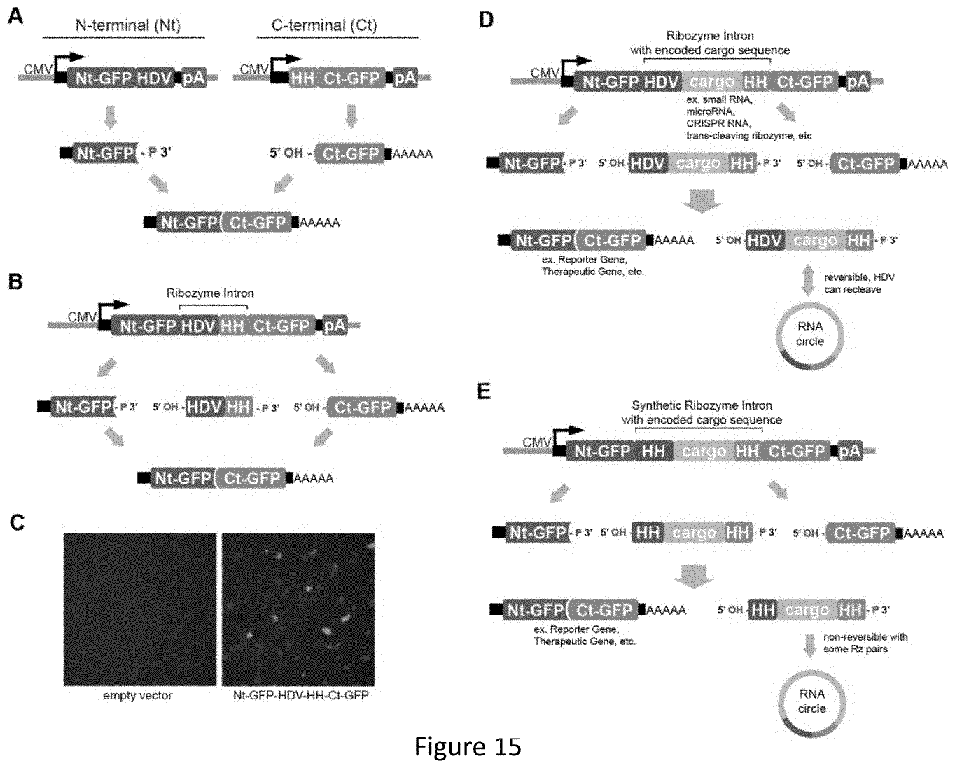

Ribozyme-mediated trans-splicing between two independent RNAs can occur when one RNA contains a 3′ ribozyme and another contains 5′ ribozyme (FIG. 15A). However, when transcribed in cis within the same RNA, it was shown that two ribozymes can mediate their own scar-less removal (FIG. 15B). This approach similarly generates two independent RNAs with 3′-P and 5′ OH termini, which can be subject to trans-splicing and translation in cells (FIG. 15B). This could also be achieved in vitro, with the addition of a ligase, such as RtcB.

The ribozyme-generated intronic sequence, also containing compatible 5′-OH and 3′-P ends, may be cis-spliced, or circularized, a common readout of RtcB ligase activity in vitro. In contrast to the lariat RNAs generated by the spliceosome during exon splicing, which are quickly degraded, RNA circles are thought to highly stable, since they no longer contain 5′ or 3′ ends and thus cannot be degraded by RNA exonucleases. Cargo sequences, which could include any number of functional or useful RNAs (such as microRNA, CRISPR guide RNA, etc), or gene expression sequences, could be inserted as ‘cargo’ between the two ribozymes (FIG. 15C). This approach could be useful for the co-delivery and expression of useful RNA sequences during ribozyme-mediated trans-splicing and expression. If one of the internal ribozymes does not require bilateral flanking sequences for activity, such as for a 5′ HDV ribozyme, the RNA circle can exist in both circular and re-cleaved linear forms (FIG. 15C). When using the VS-S in place of HDV, the system could be made inducible, requiring the delivery or expression of VS-Rz. Use of ribozymes which require bilateral flanking sequences for cleavage, such as an HH ribozyme, cleavage can be designed such that RNA circularization of the cargo RNA is unidirectional (FIG. 15D).

### Example 4: Sequences

Trans-splicing protein coding nucleic acid sequences
Nt-GFP
(SEQ ID NO: 1)
AUGGUGAGCAAGGGCGAGGAGCUGUUCACCGGGGUGGUGCCCAUCCUGGUCGAGCUGGACGGCGACGUAAACGGCCACA
AGUUCAGCGUGUCCGGCGAGGGCGAGGGCGAUGCCACCUACGGCAAGCUGACCCUGAAGUUCAUCUGCACCACCGGCAA
GCUGCCCGUGCCCUGGCCCACCCUCGUGACCACCCUGACCUACGGCGUGCAGUGCUUCAGCCGCUACCCCGACCACAUG
AAGCAGCACGACUUCUUCAAGUCCGCCAUGCCCGAAGGCUACGUCCAGGAGCGCACCAUCUUCUU
Ct-GFP
(SEQ ID NO: 2)
CAAGGACGACGGCAACUACAAGACCCGCGCCGAGGUGAAGUUCGAGGGCGACACCCUGGUGAACCGCAUCGAGCUGAAG
GGCAUCGACUUCAAGGAGGACGGCAACAUCCUGGGGCACAAGCUGGAGUACAACUACAACAGCCACAACGUCUAUAUCA
UGGCCGACAAGCAGAAGAACGGCAUCAAGGUGAACUUCAAGAUCCGCCACAACAUCGAGGACGGCAGCGUGCAGCUCGC
CGACCACUACCAGCAGAACACCCCCAUCGGCGACGGCCCCGUGCUGCUGCCCGACAACCACUACCUGAGCACCCAGUCC
GCCCUGAGCAAAGACCCCAACGAGAAGCGCGAUCACAUGGUCCUGCUGGAGUUCGUGACCGCCGCCGGGAUCACUCUCG
GCAUGGACGAGCUGUACAAGUAGUAA
Nt-Luciferase
(SEQ ID NO: 3)
AUGGAAGACGCCAAAAACAUAAAGAAAGGCCCGGCGCCAUUCUAUCCGCUGGAAGAUGGAACCGCUGGAGAGCAACUGC
AUAAGGCUAUGAAGAGAUACGCCCUGGUUCCUGGAACAAUUGCUUUUACAGAUGCACAUAUCGAGGUGGACAUCACUUA
CGCUGAGUACUUCGAAAUGUCCGUUCGGUUGGCAGAAGCUAUGAAACGAUAUGGGCUGAAUACAAAUCACAGAAUCGUC
GUAUGCAGUGAAAACUCUCUUCAAUUCUUUAUGCCGGUGUUGGGCGCGUUAUUUAUCGGAGUUGCAGUUGCGCCCGCGA
ACGACAUUUAUAAUGAACGUGAAUUGCUCAACAGUAUGGGCAUUUCGCAGCCUACCGUGGUGUUCGUUUCCAAAAAGGG
GUUGCAAAAAAUUUUGAACGUGCAAAAAAAGCUCCCAAUCAUCCAAAAAAUUAUUAUCAUGGAUUCUAAAACGGAUUAC
CAGGGAUUUCAGUCGAUGUACACGUUCGUCACAUCUCAUCUACCUCCCGGUUUUAAUGAAUACGAUUUUGUGCCAGAGU
CCUUCGAUAGGGACAAGACAAUUGCACUGAUCAUGAACUCCUCUGGAUCUACUGGUCUGCCUAAAGGUGUCGCUCUGCC
UCAUAGAACUGCCUGCGUGAGAUUCUCGCAUGCCAGAGAUCCUAUUUUUGGCAAUCAAAUCAUUCCGGAUACUGCGAUU
UUAAGUGUUGUUCCAUUCCAUCACGGUUUUGGAAUGUUUACUACACUCGGAUAUUUGAUAUGUGGAUUUCGAGUCGUCU
UAAUGUAUAGAUUUGAAGAAGAGCUGUUUCUGAGGAGCCUU
Ct-Luciferase
(SEQ ID NO: 4)
CAGGAUUACAAGAUUCAAAGUGCGCUGCUGGUGCCAACCCUAUUCUCCUUCUUCGCCAAAAGCACUCUGAUUGACAAAU
ACGAUUUAUCUAAUUUACACGAAAUUGCUUCUGGUGGCGCUCCCCUCUCUAAGGAAGUCGGGGAAGCGGUUGCCAAGAG
GUUCCAUCUGCCAGGUAUCAGGCAAGGAUAUGGGCUCACUGAGACUACAUCAGCUAUUCUGAUUACACCCGAGGGGGAU
GAUAAACCGGGCGCGGUCGGUAAAGUUGUUCCAUUUUUUGAAGCGAAGGUUGUGGAUCUGGAUACCGGGAAAACGCUGG
GCGUUAAUCAAAGAGGCGAACUGUGUGUGAGAGGUCCUAUGAUUAUGUCCGGUUAUGUAAACAAUCCGGAAGCGACCAA
CGCCUUGAUUGACAAGGAUGGAUGGCUACAUUCUGGAGACAUAGCUUACUGGGACGAAGACGAACACUUCUUCAUCGUU
GACCGCCUGAAGUCUCUGAUUAAGUACAAAGGCUAUCAGGUGGCUCCCGCUGAAUUGGAAUCCAUCUUGCUCCAACACC
CCAACAUCUUCGACGCAGGUGUCGCAGGUCUUCCCGACGAUGACGCCGGUGAACUUCCCGCCGCCGUUGUUGUUUUGGA
GCACGGAAAGACGAUGACGGAAAAAGAGAUCGUGGAUUACGUCGCCAGUCAAGUAACAACCGCGAAAAAGUUGCGCGGA
GGAGUUGUGUUUGUGGACGAAGUACCGAAAGGUCUUACCGGAAAACUCGACGCAAGAAAAAUCAGAGAGAUCCUCAUAA
AGGCCAAGAAGGGCGGAAAGAUCGCCGUGUAGUAA
NIL
(SEQ ID NO: 5)
ATGGGTCAGGCCAATACGCCCTGGAGCAGTAAGGCAAACGCGGATGCCTTTATAAATTCATTCATCAGTGCAGCATCCA
ATACTGGTTCCTTCTCTCAAGACCAAATGGAGGACATGTCACTCATCGGCAATACTCTGATGGCTGCCATGGACAATAT
GGGAGGCCGCATAACACCATCTAAGTTGCAGGCGTTGGATATGGCCTTCGCATCATCAGTGGCCGAGATCGCGGCTAGT
GAGGGCGGCGACTTGGGAGTCACTACCAACGCGATCGCGGATGCCCTCACTTCTGCTTTTTATCAAACGACCGGGGTTG
TCAATTCACGATTCATATCTGAGATCAGGAGCCTCATAGGAATGTTCGCGCAGGCTTCCGCAAATGACGTTTATGCATC
TGCTGGCTCTGGCAGCGGGGGTGGTGGGTATGGAGCCAGCTCAGCATCTGCGGCTTCTGCAAGTGCTGCTGCCCCGAGT
GGCGTAGCTTATCAGGCTCCTGCTCAGGCTCAAATCAGTTTTACGTTGCGAGGGCAACAACCTGTTTCC
AQ
(SEQ ID NO:6)
GGTCCTTATGGACCCGGTGCTAGCGCTGCGGCAGCAGCCGCTGGCGGTTATGGCCCAGGTTCAGGGCAACAGGGGCCTG
GGCAACAAGGACCTGGCCAACAAGGTCCTGGTCAGCAGGGTCCAGGGCAGCAG
NR3
(SEQ ID NO: 7)
GGCGCTGCTTCCGCTGCAGTATCAGTAGGTGGCTATGGACCTCAATCTAGTAGCGCCCCTGTTGCCTCTGCCGCCGCAT
CTCGACTTTCAAGTCCCGCCGCTAGTTCCAGGGTCAGTTCCGCGGTATCTAGCTTGGTAAGTAGCGGACCCACTAATCA
AGCGGCACTTTCAAACACAATATCCTCAGTAGTCAGTCAAGTAAGCGCATCAAACCCTGGCTTGTCAGGGTGTGACGTT
CTGGTTCAGGCACTTCTGGAAGTTGTCTCAGCGTTGGTAAGCATCCTGGGTAGCTCCTCCATAGGTCAAATTAATTATG
GCGCGAGCGCCCAATACACACAAATGGTGGGTCAGAGTGTGGCGCAGGCACTCGCAGGCGACTACAAGGATCATGACGG
AGACTATAAGGATCATGATATAGATTACAAGGACGATGATGACAAGGCCTAGTAA
Nt-4xMTS
(SEQ ID NO: 8)
AUGAGUGUGUUGACGCCGUUGCUUCUGCGAGGGCUUACCGGGUCUGCUAGAAGACUUCCGGUCCCCAGGGCCAAGAUAC
AUAGCCUCGGAGACCCGAUGUCUGUGCUCACUCCUCUGCUUUUGCGAGGACUGACUGGGUCCGCCAGACGACUCCCGGU
GCCGAGAGCUAAAAUCCAUAGCCUGGGAAAAUUGGCAACUAUGUCAGUCCUGACGCCGCUUCUUCUCCGGGGUCUUACA
GGGUCUGCAAGAAGGCUGCCUGUACCUCGGGCGAAAAUUCAUAGCUUGGGCGACCCGAUGAGUGUAUUGACGCCCCUGU
UGCUGAGAGGAUUGACUGGGUCAGCGCGCCGGCUCCCUGUCCCCCGAGCUAAGAUUCACUCCCUUGGUAAGCUGAGAAU
CCUCCAAUCAACGGUUCCGAGAGCAAGAGAUCCGCCGGUCGCCACGAGGCCUCUCGAG
Nt-DTA
(SEQ ID NO: 17)
AUGGACCCCGACGACGUGGUGGACAGCAGCAAGAGCUUCGUGAUGGAGAACUUCAGCAGCUACCACGGCACCAAGCCCG
GCUACGUGGACAGCAUCCAGAAGGGCAUCCAGAAGCCCAAGAGCGGCACCCAGGGCAACUACGACGACGACUGGAAGGG
CUUCUACAGCACCGACAACAAGUACGACGCUGCCGGCUACAGCGUGGACAACGAGAACCCCCUGAGCGGCAAGGCCGGC
GGCGUGGUGAAGGUGACCUACCCCGGCCUGACCAAGGUGCUGGCCCUGAAGGUG
Ct-DTA
(SEQ ID NO: 18)
GACAAUGCCGAGACCAUCAAGAAGGAGCUGGGCCUGAGCCUGACCGAGCCCCUGAUGGAGCAGGUGGGCACCGAGGAGU
UCAUCAAGAGAUUCGGCGACGGCGCCAGCAGAGUGGUGCUGAGCCUGCCCUUCGCCGAGGGCAGCAGCAGCGUGGAGUA
CAUCAACAACUGGGAGCAGGCCAAGGCCCUGAGCGUGGAGCUGGAGAUCAACUUCGAGACCAGAGGCAAGAGAGGCCAG
GACGCCAUGUACGAGUACAUGGCCCAGGCUUGCGCCGGCAACAGAGUGAGAAGAUAGUAA
GFPcdn (no start ATG codon)
(SEQ ID NO: 19)
GUUAGCAAGGGCGAGGAGCUCUUCACCGGGGUCGUCCCCAUCCUCGUCGAGCUCGACGGCGACGUAAACGGCCACAAGU
UCAGCGUCUCCGGCGAGGGCGAGGGCGAUGCCACCUACGGCAAGCUCACCCUGAAGUUCAUCUGCACCACCGGCAAGCU
GCCCGUGCCCUGGCCCACCCUCGUGACCACCCUGACCUACGGCGUGCAGUGCUUCAGCCGCUACCCCGACCACAUGAAG
CAGCACGACUUCUUCAAGUCCGCCAUGCCCGAAGGCUACGUCCAGGAGCGCACCAUCUUCUUCAAGGACGACGGCAACU
ACAAGACCCGCGCCGAGGUGAAGUUCGAGGGCGACACCCUGGUGAACCGCAUCGAGCUGAAGGGCAUCGACUUCAAGGA
GGACGGCAACAUCCUGGGGCACAAGCUGGAGUACAACUACAACAGCCACAACGUCUAUAUCAUGGCCGACAAGCAGAAG
AACGGCAUCAAGGUGAACUUCAAGAUCCGCCACAACAUCGAGGACGGCAGCGUGCAGCUCGCCGACCACUACCAGCAGA
ACACCCCCAUCGGCGACGGCCCCGUGCUGCUGCCCGACAACCACUACCUGAGCACCCAGUCCGCCCUGAGCAAAGACCC
CAACGAGAAGCGCGAUCACAUGGUCCUGCUGGAGUUCGUGACCGCCGCCGGGAUCACUCUCGGCAUGGACGAGCUGUAC
AAGUAG
F2-Myr
(SEQ ID NO: 20)
AUGGGUUGUUGUUUCAGCAAGACAGCGGCGAAAGGUGAAGCAGCAGCAGAAAGACCAGGCGAGGCUGCGGUAGCAUCAA
GUCCCUCCAAGGCUAAUGGGCAGGAAAACGGACACGUCAAAGUUGGAAGCGU
F2-RFP
(SEQ ID NO: 21)
AGCCAUCAUCAAGGAGUUCAUGCGCUUCAAGGUGCACAUGGAGGGCUCCGUGAACGGCCACGAGUUCGAGAUCGAGGGC
GAGGGCGAGGGCCGCCCCUACGAGGGCACCCAGACCGCCAAGCUGAAGGUGACCAAGGGUGGCCCCCUGCCCUUCGCCU
GGGACAUCCUGUCCCCUCAGUUCAUGUACGGCUCCAAGGCCUACGUGAAGCACCCCGCCGACAUCCCCGACUACUUGAA
GCUGUCCUUCCCCGAGGGCUUCAAGUGGGAGCGCGUGAUGAACUUCGAGGACGGCGGCGUGGUGACCGUGACCCAGGAC
UCCUCCCUGCAGGACGGCGAGUUCAUCUACAAGGUGAAGCUGCGCGGCACCAACUUCCCCUCCGACGGCCCCGUAAUGC
AGAAGAAGACCAUGGGCUGGGAGGCCUCCUCCGAGCGGAUGUACCCCGAGGACGGCGCCCUGAAGGGCGAGAUCAAGCA
GAGGCUGAAGCUGAAGGACGGCGGCCACUACGACGCUGAGGUCAAGACCACCUACAAGGCCAAGAAGCCCGUGCAGCUG
CCCGGCGCCUACAACGUCAACAUCAAGUUGGACAUCACCUCCCACAACGAGGACUACACCAUCGUGGAACAGUACGAAC
GCGCCGAGGGCCGCCACUCCACCGGCGGCAUGGACGAGCUGUACAAGUAGUAA
Nt-uDys
(SEQ ID NO: 22)
AUGCUUUGGUGGGAAGAAGUAGAGGACUGUUAUGAAAGAGAAGAUGUUCAAAAGAAAACAUUCACAAAAUGGGUAAAUG
CACAAUUUUCUAAGUUUGGGAAGCAGCAUAUUGAGAACCUCUUCAGUGACCUACAGGAUGGGAGGCGCCUCCUAGACCU
CCUCGAAGGCCUGACAGGGCAAAAACUGCCAAAAGAAAAAGGAUCCACAAGAGUUCAUGCCCUGAACAAUGUCAACAAG
GCACUGCGGGUUUUGCAGAACAAUAAUGUUGAUUUAGUGAAUAUUGGAAGUACUGACAUCGUAGAUGGAAAUCAUAAAC
UGACUCUUGGUUUGAUUUGGAAUAUAAUCCUCCACUGGCAGGUCAAAAAUGUAAUGAAAAAUAUCAUGGCUGGAUUGCA
ACAAACCAACAGUGAAAAGAUUCUCCUGAGCUGGGUCCGACAAUCAACUCGUAAUUAUCCACAGGUUAAUGUAAUCAAC
UUCACCACCAGCUGGUCUGAUGGCCUGGCUUUGAAUGCUCUCAUCCAUAGUCAUAGGCCAGACCUAUUUGACUGGAAUA
GUGUGGUUUGCCAGCAGUCAGCCACACAACGACUGGAACAUGCAUUCAACAUCGCCAGAUAUCAAUUAGGCAUAGAGAA
ACUACUCGAUCCUGAAGAUGUUGAUACCACCUAUCCAGAUAAGAAGUCCAUCUUAAUGUACAUCACAUCACUCUUCCAA
GUUUUGCCUCAACAAGUGAGCAUUGAAGCCAUCCAGGAAGUGGAAAUGUUGCCAAGGCCACCUAAAGUGACUAAAGAAG
AACAUUUUCAGUUACAUCAUCAAAUGCACUAUUCUCAACAGAUCACGGUCAGUCUAGCACAGGGAUAUGAGAGAACUUC
UUCCCCUAAGCCUCGAUUCAAGAGCUAUGCCUACACACAGGCUGCUUAUGUCACCACCUCUGACCCUACACGGAGCCCA
UUUCCUUCACAGCAUUUGGAAGCUCCUGAAGACAAGUCAUUUGGCAGUUCAUUGAUGGAGAGUGAAGUAAACCUGGACC
GUUAUCAAACAGCUUUAGAAGAAGUAUUAUCGUGGCUUCUUUCUGCUGAGGACACAUUGCAAGCACAAGGAGAGAUUUC
UAAUGAUGUGGAAGUGGUGAAAGACCAGUUUCAUACUCAUGAGGGGUACAUGAUGGAUUUGACAGCCCAUCAGGGCCGG
GUUGGUAAUAUUCUACAAUUGGGAAGUAAGCUGAUUGGAACAGGAAAAUUAUCAGAAGAUGAAGAAACUGAAGUACAAG
AGCAGAUGAAUCUCCUAAAUUCAAGAUGGGAAUGCCUCAGGGUAGCUAGCAUGGAAAAACAAAGCAAUUUACAUAGAGU
UUUAAUGGAUCUCCAGAAUCAGAAACUGAAAGAGUUGAAUGACUGGCUAACAAAAACAGAAGAAAGAACAAGGAAAAUG
GAGGAAGAGCCUCUUGGACCUGAUCUUGAAGACCUAAAACGCCAAGUACAACAACAUAAGGUGCUUCAAGAAGAUCUAG
AACAAGAACAAGUCAGGGUCAAUUCUCUCACUCACAUGGUGGUGGUAGUUGAUGAAUCUAGUGGAGAUCACGCAACUGC
UGCUUUGGAAGAACAACUUAAGGUAUUGGGAGAUCGAUGGGCAAACAUCUGUAGAUGGACAGAAGACCGCUGGGUUCUU
UUACAAGACAUCCUUCUCAAAUGGCAACGUCUUACUGAAGAACAGUGCCUUUUUAGUGCAUGGCUUUCAGAAAAAGAAG
AUGCAGUGAACAAGAUUCACACAACUGGCUUUAAAGAUCAAAAUGAAAUGUUAUCAAGUCUUCAAAAACUGGCCGUUUU
AAAAGCGGAUCUAGAAAAGAAAAAGCAAUCCAUGGGCAAACUGUAUUCACUCAAACAAGAUCUUCUUUCAACACUGAAG
AAUAAGUCAGUGACCCAGAAGACGGAAGCAUGGCUGGAUAACUUUGCCCGGUGUUGGGAUAAUUUAGUCCAAAAACUUG
AAAAGAGUACAGCACAGAUUUCACAGGCUGUCACCACCACUCAGCCAUCACUAACACAGACAACUGUAAUGGAAACAGU
AACUACGGUGACCACAAGGGAACAGAUCCUGGUAAAGCAUGCUCAAGAGGAACUUCCACCACCACCUCCCCAAAAGAAG
AGGCAGAUUACUGUGGAUCUUGAAAGACUCCAGGAACUUCAAGAGGCCACGGAUGAGCUGGACCUCAAGCUGCGCCAAG
CUGAGGUGAUCAAGGGAUCCUGGCAGCCCGUGGGCGAUCUCCUCAUUGACUCUCUCCAAGAUCACCUCGAGAAAGUCAA
GGCACUUCGAGGAGAAAUUGCGCCUCUGAAAGAGAACGUGAGCCAC
Ct-uDys-GFP
(SEQ ID NO: 23)
GUCAAUGACCUUGCUCGCCAGCUUACCACUUUGGGCAUUCAGCUCUCACCGUAUAACCUCAGCACUCUGGAAGACCUGA
ACACCAGAUGGAAGCUUCUGCAGGUGGCCGUCGAGGACCGAGUCAGGCAGCUGCAUGAAGCCCACAGGGACUUUGGUCC
AGCAUCUCAGCACUUUCUUUCCACGUCUGUCCAGGGUCCCUGGGAGAGAGCCAUCUCGCCAAACAAAGUGCCCUACUAU
AUCAACCACGAGACUCAAACAACUUGCUGGGACCAUCCCAAAAUGACAGAGCUCUACCAGUCUUUAGCUGACCUGAAUA
AUGUCAGAUUCUCAGCUUAUAGGACUGCCAUGAAACUCCGAAGACUGCAGAAGGCCCUUUGCUUGGAUCUCUUGAGCCU
GUCAGCUGCAUGUGAUGCCUUGGACCAGCACAACCUCAAGCAAAAUGACCAGCCCAUGGAUAUCCUGCAGAUUAUUAAU
UGUUUGACCACUAUUUAUGACCGCCUGGAGCAAGAGCACAACAAUUUGGUCAACGUCCCUCUCUGCGUGGAUAUGUGUC
UGAACUGGCUGCUGAAUGUUUAUGAUACGGGACGAACAGGGAGGAUCCGUGUCCUGUCUUUUAAAACUGGCAUCAUUUC
CCUGUGUAAAGCACAUUUGGAAGACAAGUACAGAUACCUUUUCAAGCAAGUGGCAAGUUCAACAGGAUUUUGUGACCAG
CGCAGGCUGGGCCUCCUUCUGCAUGAUUCUAUCCAAAUUCCAAGACAGUUGGGUGAAGUUGCAUCCUUUGGGGGCAGUA
ACAUUGAGCCAAGUGUCCGGAGCUGCUUCCAAUUUGCUAAUAAUAAGCCAGAGAUCGAAGCGGCCCUCUUCCUAGACUG
GAUGAGACUGGAACCCCAGUCCAUGGUGUGGCUGCCCGUCCUGCACAGAGUGGCUGCUGCAGAAACUGCCAAGCAUCAG
GCCAAAUGUAACAUCUGCAAAGAGUGUCCAAUCAUUGGAUUCAGGUACAGGAGUCUAAAGCACUUUAAUUAUGACAUCU
GCCAAAGCUGCUUUUUUUCUGGUCGAGUUGCAAAAGGCCAUAAAAUGCACUAUCCCAUGGUGGAAUAUUGCACUCCGAC
UACAUCAGGAGAAGAUGUUCGAGACUUUGCCAAGGUACUAAAAAACAAAUUUCGAACCAAAAGGUAUUUUGCGAAGCAU
CCCCGAAUGGGCUACCUGCCAGUGCAGACUGUCUUAGAGGGGGACAACAUGGAAACUGACACAAUUCUAGAGGUGAGCA
AGGGCGAGGAGCUGUUCACCGGGGUGGUGCCCAUCCUGGUCGAGCUGGACGGCGACGUAAACGGCCACAAGUUCAGCGU
GUCCGGCGAGGGCGAGGGCGAUGCCACCUACGGCAAGCUGACCCUGAAGUUCAUCUGCACCACCGGCAAGCUGCCCGUG
CCCUGGCCCACCCUCGUGACCACCCUGACCUACGGCGUGCAGUGCUUCAGCCGCUACCCCGACCACAUGAAGCAGCACG
ACUUCUUCAAGUCCGCCAUGCCCGAAGGCUACGUCCAGGAGCGCACCAUCUUCUUCAAGGACGACGGCAACUACAAGAC
CCGCGCCGAGGUGAAGUUCGAGGGCGACACCCUGGUGAACCGCAUCGAGCUGAAGGGCAUCGACUUCAAGGAGGACGGC
AACAUCCUGGGGCACAAGCUGGAGUACAACUACAACAGCCACAACGUCUAUAUCAUGGCCGACAAGCAGAAGAACGGCA
UCAAGGUGAACUUCAAGAUCCGCCACAACAUCGAGGACGGCAGCGUGCAGCUCGCCGACCACUACCAGCAGAACACCCC
CAUCGGCGACGGCCCCGUGCUGCUGCCCGACAACCACUACCUGAGCACCCAGUCCGCCCUGAGCAAAGACCCCAACGAG
AAGCGCGAUCACAUGGUCCUGCUGGAGUUCGUGACCGCCGCCGGGAUCACUCUCGGCAUGGACGAGCUGUACAAGUAA
Nt-miniDys(ΔH2-R15)
(SEQ ID NO: 129)
AUGCUUUGGUGGGAAGAAGUAGAGGACUGUUAUGAAAGAGAAGAUGUUCAAAAGAAAACAUUCACAAAAUGGGUAAAUG
CACAAUUUUCUAAGUUUGGGAAGCAGCAUAUUGAGAACCUCUUCAGUGACCUACAGGAUGGGAGGCGCCUCCUAGACCU
CCUCGAAGGCCUGACAGGGCAAAAACUGCCAAAAGAAAAAGGAUCCACAAGAGUUCAUGCCCUGAACAAUGUCAACAAG
GCACUGCGGGUUUUGCAGAACAAUAAUGUUGAUUUAGUGAAUAUUGGAAGUACUGACAUCGUAGAUGGAAAUCAUAAAC
UGACUCUUGGUUUGAUUUGGAAUAUAAUCCUCCACUGGCAGGUCAAAAAUGUAAUGAAAAAUAUCAUGGCUGGAUUGCA
ACAAACCAACAGUGAAAAGAUUCUCCUGAGCUGGGUCCGACAAUCAACUCGUAAUUAUCCACAGGUUAAUGUAAUCAAC
UUCACCACCAGCUGGUCUGAUGGCCUGGCUUUGAAUGCUCUCAUCCAUAGUCAUAGGCCAGACCUAUUUGACUGGAAUA
GUGUGGUUUGCCAGCAGUCAGCCACACAACGACUGGAACAUGCAUUCAACAUCGCCAGAUAUCAAUUAGGCAUAGAGAA
ACUACUCGAUCCUGAAGAUGUUGAUACCACCUAUCCAGAUAAGAAGUCCAUCUUAAUGUACAUCACAUCACUCUUCCAA
GUUUUGCCUCAACAAGUGAGCAUUGAAGCCAUCCAGGAAGUGGAAAUGUUGCCAAGGCCACCUAAAGUGACUAAAGAAG
AACAUUUUCAGUUACAUCAUCAAAUGCACUAUUCUCAACAGAUCACGGUCAGUCUAGCACAGGGAUAUGAGAGAACUUC
UUCCCCUAAGCCUCGAUUCAAGAGCUAUGCCUACACACAGGCUGCUUAUGUCACCACCUCUGACCCUACACGGAGCCCA
UUUCCUUCACAGCAUUUGGAAGCUCCUGAAGACAAGUCAUUUGGCAGUUCAUUGAUGGAGAGUGAAGUAAACCUGGACC
GUUAUCAAACAGCUUUAGAAGAAGUAUUAUCGUGGCUUCUUUCUGCUGAGGACACAUUGCAAGCACAAGGAGAGAUUUC
UAAUGAUGUGGAAGUGGUGAAAGACCAGUUUCAUACUCAUGAGGGGUACAUGAUGGAUUUGACAGCCCAUCAGGGCCGG
GUUGGUAAUAUUCUACAAUUGGGAAGUAAGCUGAUUGGAACAGGAAAAUUAUCAGAAGAUGAAGAAACUGAAGUACAAG
AGCAGAUGAAUCUCCUAAAUUCAAGAUGGGAAUGCCUCAGGGUAGCUAGCAUGGAAAAACAAAGCAAUUUACAUAGAGU
UUUAAUGGAUCUCCAGAAUCAGAAACUGAAAGAGUUGAAUGACUGGCUAACAAAAACAGAAGAAAGAACAAGGAAAAUG
GAGGAAGAGCCUCUUGGACCUGAUCUUGAAGACCUAAAACGCCAAGUACAACAACAUAAGGUGCUUCAAGAAGAUCUAG
AACAAGAACAAGUCAGGGUCAAUUCUCUCACUCACAUGGUGGUGGUAGUUGAUGAAUCUAGUGGAGAUCACGCAACUGC
UGCUUUGGAAGAACAACUUAAGGUAUUGGGAGAUCGAUGGGCAAACAUCUGUAGAUGGACAGAAGACCGCUGGGUUCUU
UUACAAGACAUCCUUCUCAAAUGGCAACGUCUUACUGAAGAACAGUGCCUUUUUAGUGCAUGGCUUUCAGAAAAAGAAG
AUGCAGUGAACAAGAUUCACACAACUGGCUUUAAAGAUCAAAAUGAAAUGUUAUCAAGUCUUCAAAAACUGGCCGUUUU
AAAAGCGGAUCUAGAAAAGAAAAAGCAAUCCAUGGGCAAACUGUAUUCACUCAAACAAGAUCUUCUUUCAACACUGAAG
AAUAAGUCAGUGACCCAGAAGACGGAAGCAUGGCUGGAUAACUUUGCCCGGUGUUGGGAUAAUUUAGUCCAAAAACUUG
AAAAGAGUACAGCACAGAUUUCACAGGAAAUUUCUUAUGUGCCUUCUACUUAUUUGACUGAAAUCACUCAUGUCUCACA
AGCCCUAUUAGAAGUGGAACAACUUCUCAAUGCUCCUGACCUCUGUGCUAAGGACUUUGAAGACCUCUUUAAGCAAGAG
GAGUCUCUGAAGAAUAUAAAAGAUAGUCUACAACAAAGCUCAGGUCGGAUUGACAUUAUUCAUAGCAAGAAGACAGCAG
CAUUGCAAAGUGCAACGCCUGUGGAAAGGGUGAAGCUACAGGAAGCUCUCUCCCAGCUUGAUUUCCAAUGGGAAAAAGU
UAACAAAAUGUACAAGGACCGACAAGGGCGAUUUGACAGAUCCGUUGAGAAAUGGCGGCGUUUUCAUUAUGAUAUAAAG
AUAUUUAAUCAGUGGCUAACAGAAGCUGAACAGUUUCUCAGAAAGACACAAAUUCCUGAGAAUUGGGAACAUGCUAAAU
ACAAAUGGUAUCUUAAGGAACUCCAGGAUGGCAUUGGGCAGCGGCAAACUGUUGUCAGAACAUUGAAUGCAACUGGGGA
AGAAAUAAUUCAGCAAUCCUCAAAAACAGAUGCCAGUAUUCUACAGGAAAAAUUGGGAAGCCUGAAUCUGCGGUGGCAG
GAGGUCUGCAAACAGCUGUCAGACAGAAAAAAGAGGCUAGAAGAACAAAAGAAUAUCUUGUCAGAAUUUCAAAGAGAUU
UAAAUGAAUUUGUUUUAUGGUUGGAGGAAGCAGAUAACAUUGCUAGUAUCCCACUUGAACCUGGAAAAGAGCAGCAACU
AAAAGAAAAGCUUGAGCAAGUCAAGUUACUGGUGGAAGAGUUGCCCCUGCGCCAGGGAAUCCUCAAACAAUUAAAUGAA
ACUGGAGGACCCGUGCUUGUAAGUGCUCCCAUAAGCCCAGAAGAGCAAGAUAAACUUGAAAAUAAGCUCAAGCAGACAA
AUCUCCAGUGGAUAAAGGUUUCCAGAGCUUUACCUGAGAAACAAGGAGAAAUUGAAGCUCAAAUAAAAGACCUUGGGCA
GCUUGAAAAAAAGCUUGAAGACCUUGAAGAGCAGUUAAAUCAUCUGCUGCUGUGGUUAUCUCCUAUUAGGAAUCAGUUG
GAAAUUUAUAACCAACCAAACCAAGAAGGACCAUUUGACGUUAAGGAAACUGAAAUAGCAGUUCAAGCUAAACAACCGG
AUGUGGAAGAGAUUUUGUCUAAAGGGCAGCAUUUGUACAAGGAAAAACCAGCCACUCAGCCAGUGAAGAGGAAGUUAGA
AGACCUGUCCUCUGAGUGGAAGGCGGUAAACCGUUUACUUCAAGAGCUGAGGGCAAAGCAGCCUGACCUAGCUCCUGGA
CUGACCACUAUUGGAGCCUCUCCUACUCAGACUGUUACUCUGGUGACACAACCUGUGGUUACUAAGGAAACUGCCAUCU
CCAAACUAGAAAUGCCAUCUUCCUUGAUGUUGGAGGUACCUGCUCUGGCAGAUUUCAACCGGGCUUGGACAGAACUUAC
CGACUGGCUUUCUCUGCUUGAUCAAGUUAUAAAAUCACAACGCGUGAUGGUGGGCGACCUUGAGGAUAUCAACGAGAUG
AUCAUCAAGCAGAAGGCAACAAUGCAGGAUUUGGAACAGAGGCGUCCCCAGUUGGAAGAACUCAUUACCGCUGCCCAAA
AUUUGAAAAACAAGACCAGCAAUCAAGAGGCUAGAACAAUCAUUACGGAUCGAAUUGAAAGAAUUCAGAAUCAGUGGGA
UGAAGUACAAG
Ct-miniDys(ΔH2-R15)
(SEQ ID NO: 130)
AACACCUUCAGAACCGGAGGCAACAGUUGAAUGAAAUGUUAAAGGAUUCAACACAAUGGCUGGAAGCUAAGGAAGAAGC
UGAGCAGGUCUUAGGACAGGCCAGAGCCAAGCUGGAGUCAUGGAAGGAGGGUCCCUAUACAGUAGAUGCAAUCCAAAAG
AAAAUCACAGAAACCAAGCAGUUGGCCAAAGACCUCCGCCAGUGGCAGACAAAUGUAGAUGUGGCAAAUGACUUGGCCC
UGAAACUUCUCCGGGAUUAUUCUGCAGAUGAUACCAGAAAAGUCCACAUGAUAACAGAGAAUAUCAAUGCCUCUUGGAG
AAGCAUUCAUAAAAGGGUGAGUGAGCGAGAGGCUGCUUUGGAAGAAACUCAUAGAUUACUGCAACAGUUCCCCCUGGAC
CUGGAAAAGUUUCUUGCCUGGCUUACAGAAGCUGAAACAACUGCCAAUGUCCUACAGGAUGCUACCCGUAAGGAAAGGC
UCCUAGAAGACUCCAAGGGAGUAAAAGAGCUGAUGAAACAAUGGCAAGACCUCCAAGGUGAAAUUGAAGCUCACACAGA
UGUUUAUCACAACCUGGAUGAAAACAGCCAAAAAAUCCUGAGAUCCCUGGAAGGUUCCGAUGAUGCAGUCCUGUUACAA
AGACGUUUGGAUAACAUGAACUUCAAGUGGAGUGAACUUCGGAAAAAGUCUCUCAACAUUAGGUCCCAUUUGGAAGCCA
GUUCUGACCAGUGGAAGCGUCUGCACCUUUCUCUGCAGGAACUUCUGGUGUGGCUACAGCUGAAAGAUGAUGAAUUAAG
CCGGCAGGCACCUAUUGGAGGCGACUUUCCAGCAGUUCAGAAGCAGAACGAUGUGCAUAGGGCCUUCAAGAGGGAAUUG
AAAACUAAAGAACCUGUAAUCAUGAGUACUCUUGAGACUGUACGAAUAUUUCUGACAGAGCAGCCUUUGGAAGGACUAG
AGAAACUCUACCAGGAGCCCAGAGAGCUGCCUCCUGAGGAGAGAGCCCAGAAUGUCACUCGGCUUCUACGAAAGCAGGC
UGAGGAGGUCAAUACUGAGUGGGAAAAAUUGAACCUGCACUCCGCUGACUGGCAGAGAAAAAUAGAUGAGACCCUUGAA
AGACUCCGGGAACUUCAAGAGGCCACGGAUGAGCUGGACCUCAAGCUGCGCCAAGCUGAGGUGAUCAAGGGAUCCUGGC
AGCCCGUGGGCGAUCUCCUCAUUGACUCUCUCCAAGAUCACCUGGAGAAAGUCAAGGCACUUCGAGGAGAAAUUGCGCC
UCUGAAAGAGAACGUGAGCCACGUCAAUGACCUUGCUCGCCAGCUUACCACUUUGGGCAUUCAGCUCUCACCGUAUAAC
CUCAGCACUCUGGAAGACCUGAACACCAGAUGGAAGCUUCUGCAGGUGGCCGUCGAGGACCGAGUCAGGCAGCUGCAUG
AAGCCCACAGGGACUUUGGUCCAGCAUCUCAGCACUUUCUUUCCACGUCUGUCCAGGGUCCCUGGGAGAGAGCCAUCUC
GCCAAACAAAGUGCCCUACUAUAUCAACCACGAGACUCAAACAACUUGCUGGGACCAUCCCAAAAUGACAGAGCUCUAC
CAGUCUUUAGCUGACCUGAAUAAUGUCAGAUUCUCAGCUUAUAGGACUGCCAUGAAACUCCGAAGACUGCAGAAGGCCC
UUUGCUUGGAUCUCUUGAGCCUGUCAGCUGCAUGUGAUGCCUUGGACCAGCACAACCUCAAGCAAAAUGACCAGCCCAU
GGAUAUCCUGCAGAUUAUUAAUUGUUUGACCACUAUUUAUGACCGCCUGGAGCAAGAGCACAACAAUUUGGUCAACGUC
CCUCUCUGCGUGGAUAUGUGUCUGAACUGGCUGCUGAAUGUUUAUGAUACGGGACGAACAGGGAGGAUCCGUGUCCUGU
CUUUUAAAACUGGCAUCAUUUCCCUGUGUAAAGCACAUUUGGAAGACAAGUACAGAUACCUUUUCAAGCAAGUGGCAAG
UUCAACAGGAUUUUGUGACCAGCGCAGGCUGGGCCUCCUUCUGCAUGAUUCUAUCCAAAUUCCAAGACAGUUGGGUGAA
GUUGCAUCCUUUGGGGGCAGUAACAUUGAGCCAAGUGUCCGGAGCUGCUUCCAAUUUGCUAAUAAUAAGCCAGAGAUCG
AAGCGGCCCUCUUCCUAGACUGGAUGAGACUGGAACCCCAGUCCAUGGUGUGGCUGCCCGUCCUGCACAGAGUGGCUGC
UGCAGAAACUGCCAAGCAUCAGGCCAAAUGUAACAUCUGCAAAGAGUGUCCAAUCAUUGGAUUCAGGUACAGGAGUCUA
AAGCACUUUAAUUAUGACAUCUGCCAAAGCUGCUUUUUUUCUGGUCGAGUUGCAAAAGGCCAUAAAAUGCACUAUCCCA
UGGUGGAAUAUUGCACUCCGACUACAUCAGGAGAAGAUGUUCGAGACUUUGCCAAGGUACUAAAAAACAAAUUUCGAAC
CAAAAGGUAUUUUGCGAAGCAUCCCCGAAUGGGCUACCUGCCAGUGCAGACUGUCUUAGAGGGGGACAACAUGGAAACU
CCCGUUACUCUGAUCAACUUCUGGCCAGUAGAUUCUGCGCCUGCCUCGUCCCCUCAGCUUUCACACGAUGAUACUCAUU
CACGCAUUGAACAUUAUGCUAGCAGGCUAGCAGAAAUGGAAAACAGCAAUGGAUCUUAUCUAAAUGAUAGCAUCUCUCC
UAAUGAGAGCAUAGAUGAUGAACAUUUGUUAAUCCAGCAUUACUGCCAAAGUUUGAACCAGGACUCCCCCCUGAGCCAG
CCUCGUAGUCCUGCCCAGAUCUUGAUUUCCUUAGAGAGUGAGGAAAGAGGGGAGCUAGAGAGAAUCCUAGCAGAUCUUG
AGGAAGAAAACAGGAAUCUGCAAGCAGAAUAUGACCGUCUAAAGCAGCAGCACGAACAUAAAGGCCUGUCCCCACUGCC
GUCCCCUCCUGAAAUGAUGCCCACCUCUCCCCAGAGUCCCCGGGAUGCUGAGCUCAUUGCUGAGGCCAAGCUACUGCGU
CAACACAAAGGCCGCCUGGAAGCCAGGAUGCAAAUCCUGGAAGACCACAAUAAACAGCUGGAGUCACAGUUACACAGGC
UAAGGCAGCUGCUGGAGCAACCCCAGGCAGAGGCCAAAGUGAAUGGCACAACGGUGUCCUCUCCUUCUACCUCUCUACA
GAGGUCCGACAGCAGUCAGCCUAUGCUGCUCCGAGUGGUUGGCAGUCAAACUUCGGACUCCAUGGGUGAGGAAGAUCUU
CUCAGUCCUCCCCAGGACACAAGCACAGGGUUAGAGGAGGUGAUGGAGCAACUCAACAACUCCUUCCCUAGUUCAAGAG
GAAGAAAUACCCCUGGAAAGCCAAUGAGAGAGGACACAAUGUAA
Ribozyme nucleic acid sequences for scar-less 3′ RNA Cleavage
HDV68
(SEQ ID NO: 9)
GGCCGGCAUGGUCCCAGCCUCCUCGCUGGCGCCGGCUGGGCAACAUGCUUCGGCAUGGCGAAUGGGAC
HDV68 catalytic mutant
(SEQ ID NO: 24)
5′- GGCCGGCAUGGUCCCAGCCUCCUCGCUGGCGCCGGCUGGGCAACAUGCUUCGGCAUGGUGAAUGGGAC -3′
HDV67
(SEQ ID NO: 10)
GGGUCGGCAUGGCAUCUCCACCUCCUCGCGGUCCGACCUGGGCUACUUCGGUAGGCUAAGGGAGAAG
HDV56
(SEQ ID NO: 11)
GAGGGAUAGUACAGAGCCUCCCCGUGGCUCCCUUGGAUAACCAACUGAUACUGUAC
Genomic HDV (genHDV)
(SEQ ID NO: 12)
GGCCGGCAUGGUCCCAGCCUCCUCGCUGGCGCCGGCUGGGCAACAUUCCGAGGGGACCGUCCCCUCGGUAAUGGCGAAU
GGGACCCA
Antigenomic HDV (antiHDV)
(SEQ ID NO: 13)
GGGUCGGCAUGGCAUCUCCACCUCCUCGCGGUCCGACCUGGGCAUCCGAAGGAGGACGCACGUCCACUCGGAUGGCUAA
GGGAGAGCCACU
VS Ribozyme
(SEQ ID NO: 14)
GCGGUAGUAAGCAGGGAACUCACCUCCAAUUUCAGUACUGAAAUUGUCGUAGCAGUUGACUACUGUUAUGUGAUUGGUA
GAGGCUAAGUGACGGUAUUGGCGUAAGUCAGUAUUGCAGCACAGCACAAGCCCGCUUGCGAGAAU
VS-S
(SEQ ID NO: 15)
GAAGGGCGUCGUCGCCCCGAG
VS-Rz
(SEQ ID NO: 16)
GCGGUAGUAAGCAGGGAACUCACCUCCAAUUUCAGUACUGAAAUUGUCGUAGCAGUUGACUACUGUUAUGUGAUUGGUA
GAGGCUAAGUGACGGUAUUGGCGUAAGUCAGUAUUGCAGCACAGCACAAGCCCGCUUGCGAGAAU
Hammerhead with stem 3 overhangs specific to Nt-Luc
(SEQ ID NO: 25)
5′- GAGCCUUACCGGAUGUGUUUUCCGGUCUGAUGAGUCCGGUAGCGGACGAAAGGCUC -3′
Twister with 5 nt P1 stem for Ct-Luc
(SEQ ID NO: 26)
5′- AGCCUUAACACUGCCAAUGCCGGUCCCAAGCCCGGAUAAAAGUGGAGGGAGGCU -3′
Twister with 5 nt P1 stem for Ct-Luc and T6A mutation
(SEQ ID NO: 27)
5′- AGCCUAAACACUGCCAAUGCCGGUCCCAAGCCCGGAUAAAAGUGGAGGGAGGCU -3′
Twister mutant with 5 nt P1 stem for Ct-Luc
(SEQ ID NO: 28)
5′- AGCCUUAACUCUUCCAAUGCCGGUCCCAAGCCCGGAUAAAAGUGGAGGGAGGCU -3′
Twister with 5 nt P1 stem for Ct-Luc
(SEQ ID NO: 29)
5′- AGCCUUAACACUGCCAAUGCCGGUCCCAAGCCCGGAUAAAAGUGGAGGGAGGCU -3′
Twister with 2 nt P1 stem for Ct-Luc
(SEQ ID NO: 30)
5′- AGCCUUAACACUGCCAAUGCCGGUCCCAAGCCCGGAUAAAAGUGGAGGGAG -3′
Twister with I nt P1 stem for Ct-Luc
(SEQ ID NO: 31)
5′- AGCCUUAACACUGCCAAUGCCGGUCCCAAGCCCGGAUAAAAGUGGAGGG -3′
Twister with no P1 stem for Ct-Luc
(SEQ ID NO: 32)
5′- AGCCUUAACACUGCCAAUGCCGGUCCCAAGCCCGGAUAAAAGUGGAGGG -3′
Hammerhead (HH) for 3′
(SEQ ID NO: 105)
5′ NNNNDWHACCGGAUGUGUUUUCCGGUCUGAUGAGUCCGGUAGCGGACGAAWHNNNN 3′
Twister WT with 5 nt P1 stem
(SEQ ID NO: 106)
5′ NNNNNUAACACUGCCAAUGCCGGUCCCAAGCCCGGAUAAAAGUGGAGGGNNNNN 3′
Twister Mutant with 5 nt P1 stem
(SEQ ID NO: 107)
5′ NNNNNUAACUCUUCCAAUGCCGGUCCCAAGCCCGGAUAAAAGUGGAGGGNNNNN 3′
Twister with 5 nt P1 stem with U1A mutation
(SEQ ID NO: 108)
5′ NNNNNAAACACUGCCAAUGCCGGUCCCAAGCCCGGAUAAAAGUGGAGGGNNNNN 3′
Twister with 5 nt P1 stem with U1C mutation
(SEQ ID NO: 109)
5′ NNNNNCAACACUGCCAAUGCCGGUCCCAAGCCCGGAUAAAAGUGGAGGGNNNNN 3′
Twister with 5 nt P1 stem with U1G mutation
(SEQ ID NO: 110)
5′ NNNNNGAACACUGCCAAUGCCGGUCCCAAGCCCGGAUAAAAGUGGAGGGNNNNN 3′
Ribozyme nucleic acid sequences for scar-less 5′ RNA Cleavage
Hammerhead (HH) Ribozymes with stem 1 overhangs specific to Ct-Luc
16HH
(SEQ ID NO: 33)
5′- GAAUCUUGUAAUCCUGCUGAUGAGUCCGUGAGGACGAAACGAGUAAGCUCGUC -3′
14HH
(SEQ ID NO: 34)
5′- AUCUUGUAAUCCUGCUGAUGAGUCCGUGAGGACGAAACGAGUAAGCUCGUC -3′
12HH
(SEQ ID NO: 35)
5′- CUUGUAAUCCUGCUGAUGAGUCCGUGAGGACGAAACGAGUAAGCUCGUC -3′
8HH
(SEQ ID NO: 36)
5′- UAAUCCUGCUGAUGAGUCCGUGAGGACGAAACGAGUAAGCUCGUC -3′
6HH
(SEQ ID NO: 37)
5′- AUCCUGCUGAUGAGUCCGUGAGGACGAAACGAGUAAGCUCGUC -3′
6HH Mutant
(SEQ ID NO: 38)
5′- AUCCUGCUGAUGAGUCCGUGAGGACGAGACGAGUAAGCUCGUC -3′
4HH
(SEQ ID NO: 39)
5′- CCUGCUGAUGAGUCCGUGAGGACGAAACGAGUAAGCUCGUC -3′
Hammerhead 4 nt overhang for 5′
(SEQ ID NO: 111)
5′ NNNNCUGAUGAGUCCGUGAGGACGAAACGAGUAAGCUCGUC 3′
Hammerhead 6 nt overhang for 5′
(SEQ ID NO: 112)
5′ NNNNNNCUGAUGAGUCCGUGAGGACGAAACGAGUAAGCUCGUC 3′
Hammerhead 8 nt overhang for 5′
(SEQ ID NO: 113)
5′ NNNNNNNNCUGAUGAGUCCGUGAGGACGAAACGAGUAAGCUCGUC 3′
Hammerhead 10 nt overhang for 5′
(SEQ ID NO: 114)
5′ NNNNNNNNNNCUGAUGAGUCCGUGAGGACGAAACGAGUAAGCUCGUC 3′
Hammerhead 12 nt overhang for 5′
(SEQ ID NO: 115)
5′ NNNNNNNNNNNNCUGAUGAGUCCGUGAGGACGAAACGAGUAAGCUCGUC 3′
Hammerhead 14 nt overhang for 5′
(SEQ ID NO: 116)
5′ NNNNNNNNNNNNNNCUGAUGAGUCCGUGAGGACGAAACGAGUAAGCUCGUC 3′
Hammerhead 16 nt overhang for 5′
(SEQ ID NO: 117)
5′ NNNNNNNNNNNNNNNNCUGAUGAGUCCGUGAGGACGAAACGAGUAAGCUCGUC 3′
TX2 Hammerhead 4 nt overhang for 5′ (Huang et al. 2019)
(SEQ ID NO: 118)
5′ NNNNCUGAUGAGUCCGGUAGCGGACGAAACGCGCUUCGGUGCGUC 3′
TX2 Hammerhead 6 nt overhang for 5′ (Huang et al. 2019)
(SEQ ID NO: 119)
5′ NNNNNNCUGAUGAGUCCGGUAGCGGACGAAACGCGCUUCGGUGCGUC 3′
TX2 Hammerhead 8 nt overhang for 5′ (Huang et al. 2019)
(SEQ ID NO: 120)
5′ NNNNNNNNCUGAUGAGUCCGGUAGCGGACGAAACGCGCUUCGGUGCGUC 3′
TX2 Hammerhead 10 nt overhang for 5′ (Huang et al. 2019)
(SEQ ID NO: 121)
5′ NNNNNNNNNNCUGAUGAGUCCGGUAGCGGACGAAACGCGCUUCGGUGCGUC 3′
TX2 Hammerhead 12 nt overhang for 5′ (Huang et al. 2019)
(SEQ ID NO: 122)
5′ NNNNNNNNNNNNCUGAUGAGUCCGGUAGCGGACGAAACGCGCUUCGGUGCGUC 3′
TX2 Hammerhead 14 nt overhang for 5′ (Huang et al. 2019)
(SEQ ID NO: 123)
5′ NNNNNNNNNNNNNNCUGAUGAGUCCGGUAGCGGACGAAACGCGCUUCGGUGCGUC 3′
TX2 Hammerhead 16 nt overhang for 5′ (Huang et al. 2019)
(SEQ ID NO: 124)
5′ NNNNNNNNNNNNNNNNCUGAUGAGUCCGGUAGCGGACGAAACGCGCUUCGGUGCGUC 3′
RzB Hammerhead for 5′ (Saksmerprome et al. 2004)
(SEQ ID NO: 125)
5′ NNNNNNUAANNNNNCUGAUGAGUCGCUGGGAUGCGACGAAACGCCUUCGGGCGUC 3′
RzB (Saksmerprome et al. 2004), with stem1 overhang specific to Ct-Luc
(SEQ ID NO: 40)
5′- UUGUAAUAAUCCUGCUGAUGAGUCGCUGGGAUGCGACGAAACGCCUUCGGGCGUC -3′
Splice Donor sequence for Nt vector
(SEQ ID NO: 41)
5′- GUAAGUAUCAAGGUUACAAGACAGGUUUAAGGAGACCAAUAGAAACUGGGCU -3′
Splice Acceptor sequence for Ct vector
(SEQ ID NO: 42)
5′- UGUCGAGACAGAGAAGACUCUUGCGUUUCUGAUAGGCACCUAUUGGUCUUACUGACAUCCACUUUGCCUUUCUCUC
CACAG -3′
Translational regulatory sequences for Ct vectors
GCN4 5′ UTR uORFs (Zhang and Hinnebusch 2011)
(SEQ ID NO: 43)
5′- AAACAAAAACUCACAACACAGGUUACUCUCCCCCCUAAAUUCAAAUUUUUUUUGCCCAUCAGUUUCACUAGCGAAU
UAUACAACUCACCAGCCACACAGCUCACUCAUCUACUUCGCAAUCAAAACAAAAUAUUUUAUUUUAGUUCAGUUUAUUA
AGUUAUUAUCAGUAUCGUAUUAAAAAAUUAAAGAUCAUUGAAAAAUGGCUUGCUAAACCGAUUAUAUUUUGUUUUUAAA
GUAGAUUAUUAUUAGAAAAUUAUUAAGAGAAUUAUGUGUUAAAUUUAUUGAAAGAGAAAAUUUAUUUUCCCUUAUUAAU
UAAAGUCCUUUACUUUUUUUGAAAACUGUCAGUUUUUUGAAGAGUUAUUUGUUUUGUUACCAAUUGCUAUCAUGUACCC
GUAGAAUUUUAUUCAAGAUGUUUCCGUAACGGUUACCUUUCUGUCAAAUUAUCCAGGUUUACUCGCCAAUAAAAAUUUC
CCUAUACUAUCAUUAAUUAAAUCAUUAUUAUUACUAAAGUUUUGUUUACCAAUUUGUCUGCUCAAGAAAAUAAAUUAAA
UACAAAUAAA -3′
sGCN4 5′ UTR uORFs
(SEQ ID NO: 104)
UUAAAGAUCAUUGAAAAAUGGCUUGCUAAACCGAUUAUAUUUUGUUUUUAAAGUAGAUUAUUAUUAGAAAAUUAUUAAG
AGAAUUAUGUGUUAAAUUUAUUGAAAGAGAAAAUUUAUUUUCCCUUAUUAAUUAAAGUCCUUUACUUUUUUUGAAAACU
GUCAGUUUUUUGAAGAGUUAUUUGUUUUGUUACCAAUUGCUAUCAUGUACCCGUAGAAUUUUAUUCAAGAUGUUUCCGU
AACGGUUACCU
SRY 5′ UTR uORFs (Calvo et al. 2009)
(SEQ ID NO: 44)
5′- GUUGAGGGGGUGUUGAGGGCGGAGAAAUGCAAGUUUCAUUACAAAAGUUAACGUAACAAAGAAUCUGGUAGAAAUG
AGUUUUGGAUAGUAAAAUAAGUUUCGAACUCUGGCACCUUUCAAUUUUGUCGCACUCUCCUUGUUUUUGACA -3′
Hoxa9 TIE (Leppek et al. 2020)
(SEQ ID NO: 45)
5′- GAAAAAACAGAAGAGGGAAGGAUACCAGAGCGGUUCAUACAGGGCCCAGAAACUAGGCGAGGUGACCCCUCAGCAA
GACAAACACCUCUUGAUGUUGACUGGCGAUUUUCCCCAUCUCCAGUCUGGGGAGCGGGACUAGGCAUACAGAUGAUGGA
GCUUAGAACCCGCUGGCUAGGGAAUAAAAUUCGCUGGGCAGUUUGUGCUCAAAGAAGUGGGCCAGGGCGCUUGUGACAC
AAUCAGGGCGUUUGUGACACAAACCCUUGAGGGUUGGCAGUUCUCUCCUUGGCGGUUGCUCUGGUUGCUCUGUGGGGCC
UUCCCUGUGGAGCAAGGGUGAUCUGGCCGA -3′
Hoxa3 TIE (Leppek et al. 2020)
(SEQ ID NO: 46)
5′- AGGACAAUUCGUCUCUUGGGCUGCCGAAGCGACAGCUGUCAGAGAGGCAGAAGCUUCUGGGAGCCGCGGUCUGAAG
GCUACGUGUGCUGCCUGGUCAUUCAAAGUGUCAAUUUUAGGUCCAGAAGUGUCCAAACCACAAGUUCUCAAAACUCUGA
AAAAUGGCUCCCUCC -3′
NRAS 5′UTR G-quadruplex (Kumari et al. 2007)
(SEQ ID NO: 47)
5′- CGUCCCGUGUGGGAGGGGCGGGUCUGGGUGCGGCCUGC -3′
Human IFNG 5′ UTR pseudoknot (Kaempfer 2006)
(SEQ ID NO:48 )
CACAUUGUUCUGAUCAUCUGAAGAUCAGCUAUUAGAAGAGAAAGAUCAGUUAAGUCCUUUGGACCUGAUCAGCUUGAUA
CAAGAACUACUGAUUUCAACUUCUUUGGCUUAAUUCUCUCGGAAACG
Rat ODC 5′UTR (Manzella and Blackshear 1990)
(SEQ ID NO: 49)
5′- UGUCAGUCCCUGCAGCCGCCGCCGCCGGCCGCCUUCAGUCAGCAGCUCGGCGCCACCUCCGGUCGGCGACUGCGGC
GGGCUCGACGAGGCGGCUGACGGGGCGGCGGCGGGAAGACGGCCGGGUGCGCCUUG -3′
RNA Nuclear Localization Signals
SIRLOIN RNA Nuclear Localization Signal (Lubelsky and Ulitsky 2018)
(SEQ ID NO: 50)
5′- CGCCUCCCGGGUUCAAGCGAUUCUCCUGCCUCAGCCUCCCGAGUAGCUG -3′
BORG IncRNA NLS (Zhang et al. 2014)
(SEQ ID NO: 51)
5′- ACCUCAGAAUCUACAAGUCAGCCCCAAUUAAAUGUUGUUUUA -3′
Protein Degradation Amino Acid Sequences
N- and C-terminal Protein Degradation Sequences for Nt or Ct Vectors
FKBP DD (Banaszynski et al. 2006)
(SEQ ID NO: 52)
MGVQVETISPGDGRTFPKRGQTCVVHYTGMLEDGKKVDSSRDRNKPFKFMLGKQEVIRGWEEGVAQMSVGQRAKLTISP
DYAYGATGHPGIIPPHATLVFDVELLKPE
C-terminal Protein Degradation Sequences
PEST (enhanced ODC PEST) (Li et al. 1998)
(SEQ ID NO: 53)
SHGFPPEVEEQAAGTLPMSCAQESGMDRHPAACASARINV*
ODC PEST (yeast) (Rogers et al. 1986)
(SEQ ID NO: 54)
SHGFPPEVEEQDDGTLPMSCAQESGMDRHPAACASARINV*
ODC PEST (human)
(SEQ ID NO: 55)
NPDFPPEVEEQDASTLPVSCAWESGMKRHRAACASASINV*
CL1 (Gilon et al. 1998)
(SEQ ID NO: 56)
ACKNWFSSLSHFVIHLNSHGFPPEVEEQAAGTLPMSCAQESGMDRHPAACASARINV*
CL1-PEST
(SEQ ID NO: 57)
ACKNWFSSLSHFVIHLNSHGFPPEVEEQAAGTLPMSCAQESGMDRHPAACASARINV*
E1 A PEST (Rogers et al. 1986)
(SEQ ID NO: 58)
SRECNSSTDSCDSGPSNTPPEIHPVVPLCPIKPVAVRVGGRRQAVECIEDLLNEPGQPLDLSCKRPRP*
C-myc PEST (Rogers et al. 1986)
(SEQ ID NO: 59)
LHEETPPTTSSDSEEEQEDEEEIDVVSVEKR
c-Fos PEST (Rogers et al. 1986)
(SEQ ID NO: 60)
AAHRKGSSSNEPSSDSLSSPTLLAL
v-Myb PEST (Rogers et al. 1986)
(SEQ ID NO: 61)
PSPPVDHGCLPEESASPARCMIVHQS
NPDC1 PEST
(SEQ ID NO: 62)
PPKELDTASSDEENEDGDFTVYECPGLAPTGEMEVRNPLFDHAALSAPLPAPSSPPALP
IkBa PEST (Shumway et al. 1999)
(SEQ ID NO: 63)
PESEDEESYDTESEFTEFTEDELPYDDCVFGGQRLTL
m.m. AZIN2 PEST (Lambertos and Penafiel 2019)
(SEQ ID NO: 64)
GQLLPAEEDQDAEGVCKPLSCGWEITDTLCVGPVFTPASIM*
x.1. AZIN2 PEST (Lambertos and Penafiel 2019)
(SEQ ID NO: 65)
VQLLQRGLQQTEEKENVCTPMSCGWEISDSLCFTRTFAATSII*
C-end Degrons directed by CRL2 Ubiquitin Ligases (Lin et al. 2018)
NSI
(SEQ ID NO: 66)
TSLYKKVGMGRK*
NS6
(SEQ ID NO: 67)
SLYKKVGTMAAG*
NS7
(SEQ ID NO: 68)
YKKVGTMRGRGL*
NS12
(SEQ ID NO: 69)
ERAPTGRWGRRG*
NS15
(SEQ ID NO: 70)
EGPLWHPRICGS*
SELK
(SEQ ID NO: 71)
LRGPSPPPMAGG*
SELS
(SEQ ID NO: 72)
WRPGRRGPSSGG*
C-end Degrons directed by E3 Ubiquitin Ligases (Koren et al. 2018)
EMID1
(SEQ ID NO: 73)
RDERG*
IRX6
(SEQ ID NO: 74)
GAEAG*
Ubiquitin Degrons (Chassin et al. 2019)
UbVR
(SEQ ID NO: 75)
QIFVKTLTGKTITLEVEPSDTIENVKAKIQDKEGIPPDQQRLIFAGKQLEDGRTLSDYNIQKESTLHLVLRLRGVRASA
S
2xUbVR
(SEQ ID NO: 76)
TSQIFVKTLTGKTITLEVEPSDTIENVKAKIQDKEGIPPDQQRLIFAGKQLEDGRTLSDYNIQKESTLHLVLRLRGVRA
SASQIFVKTLTGKTITLEVEPSDTIENVKAKIQDKEGIPPDQQRLIFAGKQLEDGRTLSDYNIQKESTLHLVLRLRGVR
ASAS
Sequences mimicking translation through poly A tail
12x poly K encoding tail sequence
(SEQ ID NO: 77)
AAAAAAAAAAAAAAAAAAAAAAAAAAAAAAAAAAAATAA
Translation Product 12x poly K
(SEQ ID NO: 78)
KKKKKKKKKKKK*
16x poly K encoding tail sequence
(SEQ ID NO: 79)
AAAAAAAAAAAAAAAAAAAAAAAAAAAAAAAAAAAAAAAAAAAAAAAATAA
Translation Product 16x poly K
(SEQ ID NO: 80)
KKKKKKKKKKKKKKKK*
Enzymes for enhancing or repressing ribozyme-mediated trans-splicing
Human RtcB protein sequence
(SEQ ID NO: 81)
MSRSYNDELQFLEKINKNCWRIKKGFVPNMQVEGVFYVNDALEKLMFEELRNACRGGGVGGFLPAMKQIGNVAALPGIV
HRSIGLPDVHSGYGEAIGNMAAFDMNDPEAVVSPGGVGFDINCGVRLLRTNLDESDVQPVKEQLAQAMFDHIPVGVGSK
GVIPMNAKDLEEALEMGVDWSLREGYAWAEDKEHCEEYGRMLQADPNKVSARAKKRGLPQLGTLGAGNHYAEIQVVDEI
FNEYAAKKMGIDHKGQVCVMIHSGSRGLGHQVATDALVAMEKAMKRDKIIVNDRQLACARIASPEGQDYLKGMAAAGNY
AWVNRSSMTFLTRQAFAKVFNTTPDDLDLHVIYDVSHNIAKVEQHVVDGKERTLLVHRKGSTRAFPPHHPLIAVDYQLT
GQPVLIGGTMGTCSYVLTGTEQGMTETFGTTCHGAGRALSRAKSRRNLDFQDVLDKLADMGIAIRVASPKLVMEEAPES
YKNVTDVVNTCHDAGISKKAIKLRPIAVIKG*
Human RtcB human codon optimized nucleic acid sequence
(SEQ ID NO: 82)
ATGTCCCGGTCATATAATGACGAGCTGCAATTCCTTGAGAAGATAAATAAGAATTGCTGGCGCATCAAgAAAGGCTTCG
TTCCTAATATGCAAGTTGAAGGTGTATTTTATGTAAATGACGCTTTGGAAAAGTTGATGTTCGAGGAACTGAGGAACGC
ATGTCGCGGTGGaGGEGTCGGGGGTTTTCTTCCCGCTATGAAGCAGATTGGCAATGTGGCGGCTCTGCCCGGAATTGTG
CACCGCTCTATAGGATTGCCTGACGTACACAGCGGCTACGGATTCGCCATTGGGAATATGGCGGCGTTCGATATGAACG
ACCCTGAGGCGGTTGTTAGCCCTGGAGGTGTCGGCTTCGATATAAATTGCGGAGTCAGATTGCTTCGGACAAATTTGGA
TGAATCTGACGTACAACCAGTGAAAGAGCAACTTGCACAAGCGATGTTCGATCATATTCCCGTGGGTGTGGGGTCAAAG
GGAGTAATCCCAATGAACGCGAAAGACCTGGAAGAAGCATTGGAGATGGGTGTAGACTGGTCACTGCGAGAAGGTTATG
CCTGGGCTGAAGACAAAGAGCACTGCGAGGAGTACGGTCGCATGTTGCAAGCAGACCCAAATAAAGTATCCGCGAGGGC
CAAGAAAAGAGGTTTGCCGCAGCTGGGGACATTGGGGGCCGGTAACCACTATGCAGAAATACAAGTAGTGGATGAGATT
TTCAATGAGTACGCTGCGAAGAAAATGGGGATCGACCATAAAGGTCAAGTGTGCGTAATGATACATTCTGGGAGECGCG
GACTCGGGCACCAAGTTGCAACGGACGCCCTTGTCGCCATGGAAAAAGCGATGAAGCGGGATAAAATCATCGTAAATGA
TAGGCAATTGGCTTGCGCTCGCATTGCGAGTCCGGAAGGGCAAGACTACTTGAAAGGGATGGCTGCTGCCGGGAATTAT
GCATGGGTCAACCGGAGCAGTATGACATTCTTGACGCGGCAGGCTTTTGCAAAAGTGTTTAATACGACTCCGGACGACC
TCGATCTCCATGTTATATATGATGTATCACACAATATCGCAAAGGTTGAGCAACACGTTGTGGATGGTAAGGAAAGGAC
TCTGCTGGTACACCGGAAAGGCAGTACACGGGCATTCCCGCCTCATCACCCATTGATCGCAGTCGATTATCAATTGACA
GGTCAGCCAGTTCTGATCGGAGGAACAATGGGCACATGTAGCTACGTATTGACCGGGACTGAACAGGGGATGACCGAAA
CTTTTGGCACAACATGCCATGGCGCGGGGAGGGCACTCTCCCGAGCTAAAAGTAGGAGGAATCTTGACTTCCAGGATGT
ACTGGATAAGCTgGCCGATATGGGGATAGCCATCCGGGTAGCGTCACCCAAATTGGTAATGGAGGAAGCTCCTGAAAGC
TATAAAAATGTCACTGACGTTGTCAACACATGCCATGACGCGGGTATATCCAAGAAAGCTATTAAGCTGCGCCCAATAG
CTGTAATTAAAGGATAG
E. Coli RtcB protein sequence
(SEQ ID NO: 83)
MNYELLTTENAPVKMWTKGVPVEADARQQLINTAKMPFIFKHIAVMPDVHLGKGSTIGSVIPTKGAIIPAAVGVDIGCG
MNALRTALTAEDLPENLAELRQAIETAVPHGRTTGRCKRDKGAWENPPVNVDAKWAELEAGYQWLTQKYPRFLNTNNYK
HLGTLGTGNHFIEICLDESDQVWIMLHSGSRGIGNAIGTYFIDLAQKEMQETLETLPSRDLAYFMEGTEYFDDYLKAVA
WAQLFASLNRDAMMENVVTALQSITQKTVRQPQTLAMEEINCHHNYVQKEQHFGEEIYVTRKGAVSARAGQYGIIPGSM
GAKSFIVRGLGNEESFCSCSHGAGRVMSRTKAKKLFSVEDQIRATAHVECRKDAEVIDEIPMAYKDIDAVMAAQSDLVE
VIYTLRQVVCVKG
E. Coli RtcB human codon optimized nucleic acid sequence
(SEQ ID NO: 84)
ATGAATTACGAGCTTCTTACCACTGAGAATGCACCTGTGAAAATGTGGACTAAGGGAGTGCCCGTGGAAGCGGACGCAA
GGCAGCAGCTCATAAATACAGCTAAGATGCCTTTCATCTTCAAACACATCGCGGTTATGCCCGACGTGCACCTCGGAAA
AGGCTCTACTATTGGAAGTGTGATTCCGACAAAGGGTGCGATCATACCTGCTGCCGTCGGGGTGGACATAGGCTGTGGA
ATGAATGCCCTGCGAACGGCTCTTACCGCAGAAGATCTTCCTGAGAATCTGGCCGAGCTGCGACAGGCCATTGAAACAG
CGGTTCCGCATGGTCGGACTACCGGACGGTGCAAAAGGGACAAAGGTGCGTGGGAAAACCCtCCCGTTAACGTGGATGC
GAAATGGGCTGAGTTGGAAGCAGGCTATCAATGGCTTACCCAGAAATATCCACGGTTCTTGAACACTAATAACTACAAA
CACCTGGGGACCTTGGGGACGGGGAATCATTTCATCGAAATCTGTCTTGATGAGTCTGACCAAGTGTGGATTATGCTTC
ATAGCGGTAGCCGCGGCATTGGTAACGCAATTGGGACATATTTTATTGACCTCGCGCAgAAAGAGATGCAGGAAACGCT
TGAGACGCTGCCGTCCCGAGATCTTGCGTATTTTATGGAAGGGACGGAATACTTTGACGATTATCTGAAGGCGGTAGCA
TGGGCTCAACTGTTTGCTAGTCTCAACCGAGACGCGATGATGGAAAATGTGGTAACAGCACTTCAATCAATCACCCAAA
AGACAGTGCGACAGCCCCAAACTCTCGCTATGGAAGAAATCAATTGCCACCACAATTACGTTCAgAAAGAGCAACATTT
CGGAGAAGAAATTTACGTGACAAGAAAAGGAGCTGTTAGCGCGAGGGCCGGACAGTACGGCATCATTCCTGGGTCAATG
GGTGCGAAATCTTTTATAGTACGCGGGCTTGGTAATGAAGAATCCTTCTGCAGCTGTTCTCATGGAGCCGGAAGGGTAA
TGTCCAGGACTAAGGCCAAGAAACTCTTCTCTGTGGAAGATCAAATTAGAGCTACAGCACATGTTGAATGTAGAAAGGA
TGCCGAAGTCATAGACGAGATCCCTATGGCTTACAAAGATATAGATGCTGTAATGGCTGCACAGTCAGACCTCGTAGAG
GTTATCTACACACTCCGGCAAGTCGTATGCGTAAAAGGATAG
Deinococcus radiodurans RtcB protein sequence
(SEQ ID NO: 85)
MNGKHITKLGFEGKAVGLALSAAGLREDAGVSRGDILDELRSVQNYPEQYQGGGVYADLATHLIEQQAAQQTRQSAKLR
AAPLPYRTWGEDLIEPGAHRQMDVAMQLPISRAGALMPDAHVGYGLPIGGVLATENAVIPYGVGVDIGCSMMLSVFPVA
ATGLSVDEARSLLLKHTRFGAGVGFEKRDRLDHPVLAEATWDEQPLLRHLFDKAAGQIGSSGSGNHFVEFGTFTLAQAD
PQLEGLDPGEYLAVLSHSGSRGFGAQVAGHFTNLAQRLWPALDKEAQKLAWLPLDSEAGQAYWQAMNLAGRYALANHEQ
IHARLARALGEKPLLRAQNSHNLAWKQQVNGQELIVHRKGATPAEAGQLGLIPGSMADPGYLVRGRGNPEALASASHGA
GRQLGRKAAERSLAKKDVQAYLKDRGVTLIGGGIDEAPQAYKRIEDVIARQRDLVDVLGEFRPRVVRMDTGSEDV
Deinococcus radiodurans RtcB human codon optimized nucleic acid sequence
(SEQ ID NO: 86)
ATGAACGGAAAGCACATCACGAAGTTGGGTTTCGAAGGGAAGGCTGTTGGCCTGGCATTGTCTGCGGCTGGTCTCAGGG
AAGACGCAGGCGTTTCCCGAGGAGATATTCTCGATGAACTTAGGTCTGTCCAGAATTATCCGGAGCAATATCAAGGGGG
AGGGGTCTATGCCGACTTGGCGACACACCTTATTGAGCAACAAGCTGCTCAGCAGACTAGGCAATCCGCCAAGCTGCGA
GCAGCACCACTTCCGTACCGAACGTGGGGTGAAGACCTGATCGAGCCAGGCGCACACAGACAGATGGATGTAGCAATGC
AGCTCCCGATCTCCCGGGCGGGAGCGCTGATGCCAGATGCCCACGTAGGATACGGACTTCCCATTGGAGGCGTGCTCGC
TACCGAAAACGCCGTAATCCCCTATGGAGTGGGCGTTGACATCGGTTGCTCAATGATGTTGAGTGTTTTCCCGGTGGCT
GCAACAGGTCTGTCAGTGGATGAGGCGCGGTCACTGCTTCTCAAACACACGCGCTTCGGTGCGGGGGTCGGATTCGAGA
AACGCGACAGGCTCGACCATCCTGTCTTGGCGGAGGCTACGTGGGACGAGCAGCCTTTGCTGAGACACTTGTTTGATAA
AGCTGCTGGCCAGATTGGGTCTTCCGGATCAGGGAACCACTTCGTCGAATTTGGAACTTTCACCCTCGCACAGGCCGAT
CCGCAGTTGGAAGGTTTGGAcCCTGGGGAATACTTGGCTGTTCTTTCACACTCAGGGAGTAGAGGATTTGGAGCCCAGG
TGGCTGGGCATTTTACCAACTTGGCGCAGCGCTTGTGGCCCGCACTTGATAAGGAAGCTCAAAAACTCGCATGGCTGCC
ACTGGATTCTGAGGCTGGGCAAGCcTACTGGCAAGCCATGAACTTGGCGGGACGATATGCGTTGGCTAACCATGAGCAA
ATTCACGCCCGACTGGCCCGCGCACTTGGTGAGAAGCCTCTTCTGCGCGCCCAGAACTCCCACAATCTGGCCTGGAAAC
AGCAGGTGAATGGGCAGGAATTGATAGTCCACCGCAAAGGGGCTACTCCTGCGGAAGCCGGGCAACTTGGTCTCATCCC
TGGCTCCATGGCCGACCCGGGATATTTGGTCAGGGGAAGGGGAAATCCGGAAGCATTGGCCTCTGCGTCACACGGAGCA
GGTAGACAGCTCGGCCGGAAGGCAGCGGAAAGGTCCCTGGCGAAGAAAGATGTGCAGGCTTACCTTAAAGATAGAGGAG
TAACCCTTATCGGGGGCGGGATTGACGAGGCTCCCCAGGCGTATAAAAGGATCGAAGACGTCATAGCACGCCAGCGGGA
CCTTGTGGATGTGTTGGGAGAATTTAGGCCACGAGTAGTGCGGATGGATACAGGGTCTGAAGATGTTTAG
Pyrococcus horikoshii RtcB protein sequence
(SEQ ID NO: 87)
MVVPLKRIDKIRWEIPKFDKRMRVPGRVYADEVLLEKMKNDRTLEQATNVAMLPGIYKYSIVMPDGHQGYGFPIGGVAA
FDVKEGVISPGGIGYDINCGVRLIRTNLTEKEVRPRIKQLVDTLFKNVPSGVGSQGRIKLHWTQIDDVLVDGAKWAVDN
GYGWERDLERLEEGGRMEGADPEAVSQRAKQRGAPQLGSLGSGNHFLEVQVVDKIFDPEVAKAYGLFEGQVVVMVHTGS
RGLGHQVASDYLRIMERAIRKYRIPWPDRELVSVPFQSEEGQRYFSAMKAAANFAWANRQMITHWVRESFQEVFKQDPE
GDLGMDIVYDVAHNIGKVEEHEVDGKRVKVIVHRKGATRAFPPGHEAVPRLYRDVGQPVLIPGSMGTASYILAGTEGAM
KETFGSTCHGAGRVLSRKAATRQYRGDRIRQELLNRGIYVRAASMRVVAEEAPGAYKNVDNVVKVVSEAGIAKLVARMR
PIGVAKG*
Pyrococcus horikoshii RtcB human codon optimized nucleic acid sequence
(SEQ ID NO 88)
ATGGTGGTTCCCCTGAAGAGAATAGATAAAATTCGCTGGGAGATCCCTAAGTTCGACAAAAGGATGAGAGTACCAGGAC
GGGTGTATGCAGATGAGGTCTTGCTCGAAAAAATGAAAAATGACCGCACGCTTGAACAGGCAACGAACGTCGCAATGCT
GCCAGGCATTTATAAATACAGTATTGTGATGCCCGATGGCCACCAGGGGTACGGATTTCCAATTGGAGGGGTAGCCGCT
TTCGATGTTAAAGAGGGCGTAATCAGTCCTGGTGGGATCGGGTACGACATCAATTGTGGAGTCCGACTGATCAGAACCA
ATCTCACTGAGAAAGAAGTAAGGCCCAGAATCAAGCAACTGGTTGATACTCTGTTTAAAAACGTCCCTTCTGGAGTGGG
CAGTCAAGGGCGGATTAAACTGCATTGGACTCAAATAGACGATGTACTCGTAGACGGGGCAAAATGGGCTGTGGACAAC
GGATATGGATGGGAGCGCGACCTCGAACGGTTGGAAGAAGGTGGTCGGATGGAGGGGGCCGATCCAGAGGCGGTCTCCC
AACGGGCAAAGCAGAGGGGAGCACCCCAGCTCGGGTCCCTGGGGTCTGGCAACCATTTCCTCGAAGTACAGGTCGTAGA
TAAGATCTTTGATCCTGAAGTAGCGAAAGCGTATGGCCTCTTCGAGGGGCAAGTGGTTGTGATGGTTCACACTGGTAGC
AGAGGTCTTGGGCACCAAGTTGCATCCGACTACTTGCGAATCATGGAGCGCGCAATTAGGAAGTATAGAATCCCCTGGC
CGGATAGAGAGCTTGTCTCAGTCCCTTTTCAAAGCGAGGAAGGACAAAGATACTTCAGCGCCATGAAAGCCGCGGCAAA
CTTTGCATGGGCAAATCGGCAGATGATAACTCATTGGGTACGAGAATCATTCCAAGAGGTCTTCAAACAAGATCCGGAA
GGCGACCTCGGCATGGACATTGTGTACGATGTCGCCCACAATATAGGCAAAGTGGAGGAGCACGAGGTCGATGGCAAAC
GGGTGAAAGTTATAGTCCATCGAAAGGGAGCAACTCGCGCTTTTCCACCAGGTCACGAGGCTGTACCTAGGCTGTATCG
GGATGTCGGTCAACCTGTACTCATACCCGGATCTATGGGCACAGCTTCCTATATTCTGGCTGGCACTGAAGGAGCAATG
AAAGAGACGTTTGGATCTACCTGTCACGGAGCTGGTAGGGTACTCTCCCGGAAGGCCGCGACACGACAATATCGCGGGG
ACAGGATCAGACAAGAACTTTTGAATAGAGGCATCTACGTGCGCGCCGCTAGTATGCGCGTCGTGGCCGAAGAGGCACC
TGGGGCTTACAAGAACGTGGATAACGTAGTTAAAGTAGTAAGTGAAGCCGGCATCGCCAAGCTGGTGGCCCGGATGCGC
CCGATTGGCGTGGCAAAGGGTTAG
Pyrococcus sp. ST04 RtcB protein sequence
(SEQ ID NO: 89)
MTVPLKRIDRIRWEIPKFDKRMRVPGRVYADEVLIEKMRSDRTLEQAANVAMLPGIYKYSIVMPDGHQGYGFPIGGVAA
FDVKEGVISPGGIGYDINCGVRLIRTNLTEKEVRPKIKQLVDTLFKNVPSGVGSQGRIRLHWTQIDDVLVDGAKWAVDN
GYGWERDLERLEEGGRMEGADPDAVSQRAKQRGAPQLGSLGSGNHFLEVQVVDKIYDEEVAKAYGLFEGQVVVMVHTGS
RGLGHQVASDYLRIMERAIRKYRIPWPDRELVSVPFQSEEGQRYFSAMKAAANFAWANRQMITHWVSRESFQEVFQDPE
GDLGMDIVYDVAHNIGKVEEHEVDGKKVTVIVHRKGATRAFPPGHEAIPRIYRDVGQPVLIPGSMGTASYVLAGTEGAM
KETFGSTCHGAGRVLSRKAATRQYRGDRIRNELLQRGIYVRAASMRVVAEEAPGAYKNVDNVVKVVSEAGIAKLVARMR
PIGVAKG*
Pyrococcus sp. ST04 RtcB human codon optimized nucleic acid sequence
(SEQ ID NO: 90)
ATGACCGTTCCCCTGAAGAGAATAGATAGGATTCGCTGGGAGATCCCTAAGTTCGACAAAAGGATGAGAGTACCAGGAC
GGGTGTATGCAGATGAGGTCTTGATCGAGAAAATGAGAAGCGACCGCACGCTTGAACAGGCAGCCAACGTCGCAATGCT
GCCAGGCATTTATAAATACAGTATTGTGATGCCCGATGGCCACCAGGGGTACGGATTTCCAATTGGAGGGGTAGCCGCT
TTCGATGTTAAAGAGGGCGTAATCAGTCCTGGTGGGATCGGGTACGACATCAATTGTGGAGTCCGACTGATCAGAACCA
ATCTCACTGAGAAAGAAGTAAGGCCCAAAATCAAGCAACTGGTTGATACTCTGTTTAAAAACGTCCCTTCTGGAGTGGG
CAGTCAAGGGCGGATTAGACTGCATTGGACTCAAATAGACGATGTACTCGTAGACGGGGCAAAATGGGCTGTGGACAAC
GGATATGGATGGGAGCGCGACCTCGAACGGTTGGAAGAAGGTGGTCGGATGGAGGGGGCCGATCCAGACGCGGTCTCCC
AACGGGCAAAGCAGAGGGGAGCACCCCAGCTCGGGTCCCTGGGGTCTGGCAACCATTTCCTCGAAGTACAGGTCGTAGA
TAAGATCTACGATGAGGAAGTAGCGAAAGCGTATGGCCTCTTCGAGGGGCAAGTGGTTGTGATGGTTCACACTGGTAGC
AGAGGTCTTGGGCACCAAGTTGCATCCGACTACTTGCGAATCATGGAGCGCGCAATTAGGAAGTATAGAATCCCCTGGC
CGGATAGAGAGCTTGTCTCAGTCCCTTTTCAAAGCGAGGAAGGACAAAGATACTTCAGCGCCATGAAAGCCGCGGCAAA
CTTTGCATGGGCAAATCGGCAGATGATAACTCATTGGGTACGAGAATCATTCCAAGAGGTCTTCAGACAAGATCCGGAA
GGCGACCTCGGCATGGACATTGTGTACGATGTCGCCCACAATATAGGCAAAGTGGAGGAGCACGAGGTCGATGGCAAGA
AAGTGACCGTTATAGTCCATCGAAAGGGAGCAACTCGCGCTTTTCCACCAGGTCACGAGGCTATCCCTAGGATCTATCG
GGATGTCGGTCAACCTGTACTCATACCCGGATCTATGGGCACAGCTTCCTATGTGCTGGCTGGCACTGAAGGAGCAATG
AAAGAGACGTTTGGATCTACCTGTCACGGAGCTGGTAGGGTACTCTCCCGGAAGGCCGCGACACGACAATATCGCGGGG
ACAGGATCAGAAATGAACTTTTGCAAAGAGGCATCTACGTGCGCGCCGCTAGTATGCGCGTCGTGGCCGAAGAGGCACC
TGGGGCTTACAAGAACGTGGATAACGTAGTTAAAGTAGTAAGTGAAGCCGGCATCGCCAAGCTGGTGGCCCGGATGCGC
CCGATTGGCGTGGCAAAGGGTTAG
Thermococcus sp. EP1 RtcB protein sequence
(SEQ ID NO: 91)
MEIPLKRLDKIRWEIPKFNRRMRVPGRVYADDTLLQKMRQDKTLEQATNVAMLPGIYKYSIVMPDGHQGYGFPIGGVAA
FDVKEGVISPGGVGYDINCGVRLIRTNLVEKEVRPKIKQLIDTLFKNVPSGLGSKGRIRLHWTQLDDVLADGAKWAVDN
GYGWKDDLEHLEEGGRMEGANPNAVSQKAKQRGAPQLGSLGSGNHFLEIQVVDKVFNEEIAKAYGLFEGQIVVMVHTGS
RGLGHQVASDYLRIMEKANRKYNVPWPDRELVSVPFQTEEGQRYFSAMKAAANFAWANRQMITHWVRESFEEVFKQKAE
DLGMHIVYDVAHNIAKVEEHEVNGRKIKVVVHRKGATRAFPAGHEAIPKAYRDVGQPVLIPGSMGTASYVLAGAEGSMR
ETFGSTCHGAGRVLSRHAATRQFRGDRLRNELMQRGIYIRAASMRVVAEEAPGAYKNVDNVVRVVHEAGIANLVARMRP
IGVAKG*
Thermococcus sp. EP1 RtcB human codon optimized nucleic acid sequence
(SEQ ID NO: 92)
ATGGAGATACCACTCAAACGACTTGACAAGATCCGATGGGAGATTCCCAAATTTAACAGACGAATGAGAGTTCCGGGAA
GAGTTTACGCAGATGATACATTGCTCCAAAAgATGCGACAAGATAAGACGCTCGAaCAAGCCACCAACGTGGCCATGCT
CCCAGGCATTTATAAGTATAGTATAGTCATGCCTGACGGACACCAGGGTTATGGATTCCCGATTGGCGGTGTAGCAGCC
TTCGACGTAAAAGAGGGAGTAATTAGTCCTGGCGGTGTTGGTTATGATATTAACTGTGGCGTGAGGCTTATCAGGACGA
ATCTTGTAGAGAAGGAAGTGCGACCAAAAATCAAACAACTTATAGATACTTTGTTCAAAAATGTCCCGTCTGGGCTCGG
ATCAAAGGGTCGGATAAGGCTCCACTGGACTCAACTGGATGATGTTCTGGCTGATGGGGCAAAATGGGCTGTTGACAAT
GGGTACGGGTGGAAGGATGATCTCGAACATTTGGAGGAGGGCGGACGGATGGAGGGCGCAAACCCCAATGCCGTTTCAC
AGAAAGCGAAGCAAAGGGGAGCGCCACAGCTTGGGTCCCTTGGCTCAGGCAATCATTTCCTCGAAATTCAGGTCGTCGA
TAAGGTTTTTAACGAAGAGATAGCAAAGGCTTACGGACTCTTTGAAGGTCAGATAGTGGTAATGGTCCATACGGGCTCT
CGGGGACTGGGACATCAAGTCGCAAGTGACTACCTGAGGATCATGGAGAAAGCCAATCGCAAGTACAATGTGCCCTGGC
CTGACCGGGAGCTTGTTAGCGTGCCCTTCCAGACGGAAGAGGGTCAACGATACTTTAGCGCTATGAAGGCGGCAGCTAA
TTTCGCTTGGGCAAACAGACAGATGATAACACATTGGGTTAGAGAGTCCTTCGAGGAGGTCTTTAAACAAAAAGCTGAG
GACCTTGGAATGCATATTGTCTATGATGTTGCCCATAACATAGCAAAAGTAGAGGAACATGAGGTGAACGGGCGGAAAA
TTAAGGTCGTAGTACACAGAAAAGGCGCTACCAGAGCATTCCCCGCAGGACACGAGGCCATACCCAAAGCATATAGAGA
TGTCGGCCAGCCAGTgCTCATACCGGGATCTATGGGTACGGCGTCCTATGTCTTGGCGGGTGCTGAAGGATCAATGAGG
GAGACGTTCGGCTCAACCTGTCATGGGGCAGGTCGGGTCTTGTCTCGGCATGCTGCAACTCGGCAGTTCCGCGGGGATC
GACTCAGGAATGAACTCATGCAGAGAGGCATTTACATACGCGCTGCCTCCATGCGCGTTGTCGCCGAGGAAGCECCCGG
CGCCTATAAGAACGTAGACAATGTCGTCAGGGTGGTGCATGAAGCGGGAATTGCGAACTTGGTAGCCAGGATGCGCCCA
ATAGGGGTTGCCAAGGGATAGTAA
Human Archease protein sequence
(SEQ ID NO: 93)
MAQEEEDVRDYNLTEEQKAIKAKYPPVNRKYEYLDHTADVQLHAWGDTLEEAFEQCAMAMFGYMTDTGTVEPLQTVEVE
TQGDDLQSLLFHFLDEWLYKFSADEFFIPREVKVLSIDQRNFKLRSIGWGEEFSLSKHPQGTEVKAITYSAMQVYNEEN
PEVFVIIDI*
Human Archease human codon optimized nucleic acid sequence
(SEQ ID NO: 94)
AGGAACAAAAGGCCATCAAAGCGAAATATCCGCCTGTAAACCGAAAGTATGAGTACCTGGATCACACTGCGGACGTCCA
GTTGCATGCCTGGGGCGACACTCTGGAGGAGGCATTCGAACAATGTGCAATGGCAATGTTTGGCTACATGACTGATACA
GGCACAGTGGAGCCCCTTCAAACGGTAGAGGTAGAAACTCAGGGAGALGATCTTCAGAGCTTGCTCTTCCATTTTCTCG
ACGAATGGTTGTATAAGTTCAGTGCCGACGAGTTCTTCATTCCACGCGAAGTGAAAGTGCTGAGTATTGATCAGAGAAA
CTTTAAACTTAGGTCTATTGGGTGGGGTGAAGAGTTCTCTTTGTCTAAACACCCTCAAGGAACTGAGGTAAAGGCGATA
ACTTACTCAGCCATGCAGGTATATAACGAGGAGAATCCTGAGGTTTTCGTAATCATTGATATATAG
Pyrococcus horikoshii Archease protein sequence
(SEQ ID NO: 95)
MKKWEHYEHTADIGIRGYGDSLEEAFEAVAIALFDVMVNVNKVEKKEVREIEVEAEDLEALLYSFLEELLVIHDIEGLV
FRDFEVKIERVNGKYRLRAKAYGEKLDLKKHEPKEEVKAITYHDMKIERLPNGKWMAQLVPDI*
Pyrococcus horikoshii Archease human codon optimized nucleic acid sequence
(SEQ ID NO: 96)
ATGAAGAAATGGGAGCACTATGAGCATACTGCCGACATTGGTATTCGGGGATATGGGGATAGCCTTGAGGAGGCATTCG
AAGCAGTAGCCATCGCGCTCTTTGATGTAATGGTGAACGTGAATAAAGTCGAGAAGAAGGAAGTCCGAGAAATTGAAGT
GGAGGCAGAAGATTTGGAGGCCCTCCTTTATTCATTCCTGGAAGAACTGTTGGTTATTCATGATATAGAGGGACTGGTT
TTCAGGGACTTTGAAGTTAAGATAGAGAGAGTAAATGGCAAATACCGACTTCGAGCGAAAGCCTACGGTGAGAAGCTCG
ACCTCAAGAAGCACGAACCGAAAGAGGAAGTAAAGGCGATAACCTACCATGATATGAAAATTGAACGGTTGCCCAATGG
AAAGTGGATGGCTCAACTCGTTCCAGATATTTAG
T4 Polynucleotide Kinase (T4 PNK) protein sequence
(SEQ ID NO: 97)
MKKIILTIGCPGSGKSTWAREFIAKNPGFYNINRDDYRQSIMAHEERDEYKYTKKKEGIVTGMQFDTAKSILYGGDSVK
GVIISDTNLNPERRLAWETFAKEYGWKVEHKVFDVPWTELVKRNSKRGTKAVPIDVLRSMYKSMREYLGLPVYNGTPGK
PKAVI FDVDGTLAKMNGRGPYDLEKCDTDVINPMWELSKMYALMGYQIVWSGRESGTKEDPTKYYRMTRKWVEDIAG
VPLVMQCQREQGDTRKDDVVKEEIFWKHIAPHFDVKLAIDDRTQVVEMWRRIGVECWQVASGDF*
T4 PNK human codon optimized nucleic acid sequence
(SEQ ID NO: 98)
ATGAAGAAAATTATACTTACAATCGGATGCCCTGGTAGTGGTAAGAGCACTTGGGCGAGGGAATTTATTGCGAAgAACC
CtGGATTTTATAATATCAATCGAGACGACTACCGGCAGTCTATTATGGCCCACGAGGAACGAGACGAATACAAGTATAC
CAAGAAGAAAGAAGGGATTGTCACGGGTATGCAATTTGACACCGCCAAATCAATACTGTACGGAGGTGATTCAGTCAAA
GGCGTTATCATATCAGACACTAACCTCAATCCTGAACGCCGATTGGCATGGGAAACATTTGCGAAGGAATACGGTTGGA
AGGTTGAACACAAGGTGTTCGATGTCCCGTGGACCGAACTGGTAAAACGCAATTCTAAACGAGGCACTAAAGCTGTGCC
CATTGACGTACTTCGAAGTATGTACAAGTCCATGAGAGAGTACCTGGGGCTTCCCGTCTATAACGGTACGCCGGGCAAA
CCGAAGGCGGTGATCTTTGACGTAGATGGGACTCTGGCGAAGATGAATGGTCGCGGACCATACGATTTGGAAAAATGTG
ACACAGATGTAATCAACCCAATGGTAGTAGAGCTTAGCAAGATGTACGCATTGATGGGcTACCAAATTGTCGTGGTGTC
CGGGCGGGAGTCAGGCACAAAAGAAGATCCGACGAAGTATTATCGCATGACACGGAAATGGGTCGAAGATATAGCCGGG
GTgCCTCTCGTTATGCAATGTCAACGAGAACAGGGCGACACACGGAAGGATGACGTAGTGAAGGAGGAAATTTTCTGGA
AGCATATAGCGCCACACTTTGACGTTAAGCTCGCCATCGACGACCGAACTCAGGTGGTCGAGATGTGGCGACGAATTGG
CGTAGAGTGTTGGCAAGTTGCATCTGGAGATTTTTAG
E. Coli thpR protein sequence
(SEQ ID NO: 99)
MSEPQRLFFAIDLPAEIREQIIHWRATHFPPEAGRPVAADNLHLTLAFLGEVSAEKEKALSLLAGRIRQPGFTLTLDDA
GQWLRSRWWLGMRQPPRGLIQLANMLRSQAARSGCFQSNRPFHPHITLLRDASEAVTIPPPGFNWSYAVTEFTLYASS
FARGRTRYTPLKRWALTQ*
E. Coli thpR human codon optimized nucleic acid sequence
(SEQ ID NO: 100)
ATGAGTGAGCCTCAACGATTGTTCTTTGCCATAGATTTGCCTGCTGAAATTAGAGAGCAAATTATCCATTGGAGAGCCA
CCCATTTCCCCCCAGAAGCTGGACGACCAGTCGCAGCGGACAACCTCCACCTTACACTGGCGTTCTTGGGTGAAGTGAG
CGCCGAGAAAGAGAAAGCTCTCTCACTTCTGGCTGGGAGGATTCGGCAGCCGGGCTTTACCCTTACTCTGGATGATGCC
GGCCAGTGGCTGAGGTCCAGGGTTGTCTGGCTCGGAATGAGGCAACCACCTAGGGGGCTCATCCAGCTCGCCAATATGC
TGAGATCCCAGGCCGCAAGGTCTGGCTGCTTCCAATCAAACAGGCCATTCCACCCGCATATTACCTTGCTCAGAGATGC
CTCCGAGGCAGTAACTATTCCACCTCCCGGCTTTAACTGGAGTTACGCCGTCACAGAATTTACTCTGTACGCCTCCAGC
TTCGCCCGAGGGAGAACCAGGTACACGCCTTTGAAGCGGTGGGCCTTGACCCAGTAG
Human PNKP protein sequence
(SEQ ID NO: 101)
MGEVEAPGRLWLESPPGGAPPIFLPSDGQALVLGRGPLTQVTDRKCSRTQVELVADPETRTVAVKQLGVNPSTTGTQEL
KPGLEGSLGVGDTLYLVNGLHPLTLRWEETRTPESQPDTPPGTPLVSQDEKRDAELPKKRMRKSNPGWENLEKLLVFTA
AGVKPQGKVAGFDLDGTLITTRSGKVFPTGPSDWRILYPEIPRKLRELEAEGYKLVIFTNQMSIGRGKLPAEEFKAKVE
AVVEKLGVPFQVLVATHAGLYRKPVTGMWDHLQEQANDGTPISIGDSIFVGDAAGRPANWAPGRKKKDFSCADRLFALN
LGLPFATPEEFFLKWPAAGFELPAFDPRTVSRSGPLCLPESRALLSASPEVVVAVGFPGAGKSTFLKKHLVSAGYVHVN
RDTLGSWQRCVTTCETALKQGKRVAIDNTNPDAASRARYVQCARAAGVPCRCFLFTATLEQARHNNRFREMTDSSHIPV
SDMVMYGYRKQFEAPTLAEGFSAILEIPFRLWVEPRLGRLYCQFSEG*
Human PNKP human codon optimized nucleic acid sequence
(SEQ ID NO: 102)
ATGGGCGAGGTGGAGGCCCCGGGCCGCTTGTGGCTCGAGAGCCCCCCTGGGGGAGCGCCCCCCATCTTCCTGCCCTCGG
ACGGGCAAGCCCTGGTCCTGGGCAGGGGACCCCTGACCCAGGTTACGGACCGGAAGTGCTCCAGAACTCAAGTGGAGCT
GGTCGCAGATCCTGAGACCCGGACAGTGGCAGTGAAACAGCTGGGAGTTAACCCCTCAACTACCGGGACCCAGGAGTTG
AAGCCGGGGTTGGAGGGCTCTCTGGGGGTGGGGGACACACTGTATTTGGTCAATGGCCTCCACCCACTGACCCTGCGCT
GGGAAGAGACCCGCACACCAGAATCCCAGCCAGATACTCCGCCTGGCACCCCTCTGGTGTCCCAAGATGAGAAGAGAGA
TGCTGAGCTGCCGAAGAAGCGTATGCGGAAGTCAAACCCCGGCTGGGAGAACTTGGAGAAGTTGCTAGTGTTCACCGCA
GCTGGGGTGAAACCCCAGGGCAAGGTGGCTGGCTTTGATCTGGACGGGACGCTCATCACCACACGCTCTGGGAAGGTCT
TTCCCACTGGCCCCAGTGACTGGAGGATCTTGTACCCAGAGATTCCCCGTAAGCTCCGAGAGCTGGAAGCCGAGGGCTA
CAAGCTGGTGATCTTCACCAACCAGATGAGCATCGGGCGCGGGAAGCTGCCAGCCGAGGAGTTCAAGGCCAAGGTGGAG
GCTGTGGTGGAGAAGCTGGGGGTCCCCTTCCAGGTGCTGGTGGCCACGCACGCAGGCTTGTACCGGAAGCCGGTGACGG
GCATGTGGGACCATCTGCAGGAGCAGGCCAACGACGGCACGCCCATATCCATCGGGGACAGCATCTTTGTGGGAGACGC
AGCCGGACGCCCGGCCAACTGGGCCCCGGGGCGGAAGAAGAAAGACTTCTCCTGCGCCGATCGCCTGTTTGCCCTCAAC
CTTGGCCTGCCCTTCGCCACGCCTGAGGAGTTCTTTCTCAAGTGGCCAGCAGCCGGCTTCGAGCTCCCAGCCTTTGATC
CGAGGACTGTCTCCCGCTCAGGGCCTCTCTGCCTCCCCGAGTCCAGGGCCCTCCTGAGCGCCAGCCCGGAGGTGGTTGT
CGCAGTGGGATTCCCTGGGGCCGGGAAGTCCACCTTTCTCAAGAAGCACCTCGTGTCGGCCGGATATGTCCACGTGAAC
AGGGACACGCTAGGCTCCTGGCAGCGCTGTGTGACCACGTGTGAGACAGCCCTGAAGCAAGGGAAACGGGTCGCCATCG
ACAACACAAACCCAGACGCCGCGAGCCGCGCCAGGTACGTCCAGTGTGCCCGAGCCGCGGGCGTCCCCTGCCGCTGCTT
CCTCTTCACCGCCACTCTGGAGCAGGCGCGCCACAACAACCGGTTTCGAGAGATGACGGACTCCTCTCATATCCCCGTG
TCAGACATGGTCATGTATGGCTACAGGAAGCAGTTCGAGGCCCCAACGCTGGCTGAAGGCTTCTCTGCCATCCTGGAGA
TCCCGTTCCGGCTATGGGTGGAGCCGAGGCTGGGGCGGCTGTACTGCCAGTTCTCCGAGGGCTAG
GFP with internal synthetic ribozyme intron with and without cargo
NtGFP-HDV-HH-CtGFP
(SEP ID NO: 103)
AUGGUGAGCAAGGGCGAGGAGCUGUUCACCGGGGUGGUGCCCAUCCUGGUCGAGCUGGACGGCGACGUAAACGGCCACA
AGUUCAGCGUGUCCGGCGAGGGCGAGGGCGAUGCCACCUACGGCAAGCUGACCCUGAAGUUCAUCUGCACCACCGGCAA
GCUGCCCGUGCCCUGGCCCACCCUCGUGACCACCCUGACCUACGGCGUGCAGUGCUUCAGCCGCUACCCCGACCACAUG
AAGCAGCACGACUUCUUCAAGUCCGCCAUGCCCGAAGGCUACGUCCAGGAGCGCACCAUCUUCUUggccggcauggucc
cagccuccucgcuggcgccggcugggcaacaugcuucggcauggcgaaugggaccccgggacauaacuaguuaaaccaa
auccuugcugaugaguccgugaggacgaaacgaguaagcucgucCAAGGACGACGGCAACUACAAGACCCGCGCCGAGG
UGAAGUUCGAGGGCGACACCCUGGUGAACCGCAUCGAGCUGAAGGGCAUCGACUUCAAGGAGGACGGCAACAUCCUGGG
GCACAAGCUGGAGUACAACUACAACAGCCACAACGUCUAUAUCAUGGCCGACAAGCAGAAGAACGGCAUCAAGGUGAAC
UUCAAGAUCCGCCACAACAUCGAGGACGGCAGCGUGCAGCUCGCCGACCACUACCAGCAGAACACCCCCAUCGGCGACG
GCCCCGUGCUGCUGCCCGACAACCACUACCUGAGCACCCAGUCCGCCCUGAGCAAAGACCCCAACGAGAAGCGCGAUCA
CAUGGUCCUGCUGGAGUUCGUGACCGCCGCCGGGAUCACUCUCGGCAUGGACGAGCUGUACAAGUAG
NtGFP-HDV-CARGO-HH-CtGFP
(SEQ ID NO: 126)
AUGGUGAGCAAGGGCGAGGAGCUGUUCACCGGGGUGGUGCCCAUCCUGGUCGAGCUGGACGGCGACGUAAACGGCCACA
AGUUCAGCGUGUCCGGCGAGGGCGAGGGCGAUGCCACCUACGGCAAGCUGACCCUGAAGUUCAUCUGCACCACCGGCAA
GCUGCCCGUGCCCUGGCCCACCCUCGUGACCACCCUGACCUACGGCGUGCAGUGCUUCAGCCGCUACCCCGACCACAUG
AAGCAGCACGACUUCUUCAAGUCCGCCAUGCCCGAAGGCUACGUCCAGGAGCGCACCAUCUUCUUggccggcauggucc
cagccuccucgcuggcgccggcugggcaacaugcuucggcauggcgaaugggacNuccuugcugaugaguccgugagga
cgaaacgaguaagcucgucCAAGGACGACGGCAACUACAAGACCCGCGCCGAGGUGAAGUUCGAGGGCGACACCCUGGU
GAACCGCAUCGAGCUGAAGGGCAUCGACUUCAAGGAGGACGGCAACAUCCUGGGGCACAAGCUGGAGUACAACUACAAC
AGCCACAACGUCUAUAUCAUGGCCGACAAGCAGAAGAACGGCAUCAAGGUGAACUUCAAGAUCCGCCACAACAUCGAGG
ACGGCAGCGUGCAGCUCGCCGACCACUACCAGCAGAACACCCCCAUCGGCGACGGCCCCGUGCUGCUGCCCGACAACCA
CUACCUGAGCACCCAGUCCGCCCUGAGCAAAGACCCCAACGAGAAGCGCGAUCACAUGGUCCUGCUGGAGUUCGUGACC
GCCGCCGGGAUCACUCUCGGCAUGGACGAGCUGUACAAGUAG
NtGFP-HDV
(SEQ ID NO: 127)
AUGGUGAGCAAGGGCGAGGAGCUGUUCACCGGGGUGGUGCCCAUCCUGGUCGAGCUGGACGGCGACGUAAACGGCCACA
AGUUCAGCGUGUCCGGCGAGGGCGAGGGCGAUGCCACCUACGGCAAGCUGACCCUGAAGUUCAUCUGCACCACCGGCAA
GCUGCCCGUGCCCUGGCCCACCCUCGUGACCACCCUGACCUACGGCGUGCAGUGCUUCAGCCGCUACCCCGACCACAUG
AAGCAGCACGACUUCUUCAAGUCCGCCAUGCCCGAAGGCUACGUCCAGGAGCGCACCAUCUUCUUggccggcauggucc
cagccuccucgcuggcgccggcugggcaacaugcuucggcauggcgaaugggac
HH-CtGFP
(SEQ ID NO: 128)
uccuugcugaugaguccgugaggacgaaacgaguaagcucgucCAAGGACGACGGCAACUACAAGACCCGCGCCGAGGU
GAAGUUCGAGGGCGACACCCUGGUGAACCGCAUCGAGCUGAAGGGCAUCGACUUCAAGGAGGACGGCAACAUCCUGGGG
CACAAGCUGGAGUACAACUACAACAGCCACAACGUCUAUAUCAUGGCCGACAAGCAGAAGAACGGCAUCAAGGUGAACU
UCAAGAUCCGCCACAACAUCGAGGACGGCAGCGUGCAGCUCGCCGACCACUACCAGCAGAACACCCCCAUCGGCGACGG
CCCCGUGCUGCUGCCCGACAACCACUACCUGAGCACCCAGUCCGCCCUGAGCAAAGACCCCAACGAGAAGCGCGAUCAC
AUGGUCCUGCUGGAGUUCGUGACCGCCGCCGGGAUCACUCUCGGCAUGGACGAGCUGUACAAGUAG

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A system for generating an RNA molecule encoding a protein of interest comprising:
a nucleic acid molecule encoding a first RNA molecule comprising a coding region encoding a first portion of the protein of interest and a 3′ribozyme; and
a nucleic acid molecule encoding a second RNA molecule comprising a coding region encoding a second portion of the protein of interest and a 5′ribozyme.

2. The system of claim 1, wherein the 3′ribozyme catalyzes itself out of the first RNA molecule, thereby generating a 3′P or 2′3′ cP end.

3. The system of any of claims 1-2, wherein the 5′ribozyme catalyzes itself out of the second RNA molecule, thereby generating a 5′0H end.

4. The system of claim 3, wherein the 3′P or 2′3′ cP end is ligated to the 5′0H end to form an RNA molecule comprising the coding region of the first RNA molecule and the coding region of the second RNA molecule.

5. The system of any of claims 1-4 wherein the 3′ ribozyme is a member of the HDV family of ribozymes.

6. The system of any of claims 1-4 wherein the 5′ ribozyme is a member of the HH family of ribozymes.

7. The system of any of claims 1-6, wherein the system further comprises one or more additional nucleic acid molecules encoding one or more additional RNA molecules, each additional RNA molecule comprising a coding region encoding a domain of the protein of interest; a 5′ ribozyme; and a 3′ ribozyme.

8. The system of any of claims 1-6, wherein the system further comprises one or more additional nucleic acid molecules encoding one or more additional RNA molecules, each additional RNA molecule comprising a coding region encoding a domain of the protein of interest; a 5′ ribozyme; and a 3′ ribozyme recognition sequence.

9. The system of claim 8, wherein the system further comprises a ribozyme that interacts with the 3′ ribozyme recognition sequence which induces the removal of the 3′ recognition sequence.

10. The system of claim 9, wherein the 3′ ribozyme recognition sequence comprises VS-S and wherein the ribozyme is VS-Rz.

11. A method for generating an RNA molecule encoding a protein of interest comprising:
administering to a cell or tissue a nucleic acid molecule encoding a first RNA molecule comprising a coding region encoding a first portion of the protein of interest and a 3′ribozyme; and
administering to a cell or tissue a nucleic acid molecule encoding a second RNA molecule comprising a coding region encoding a second portion of the protein of interest and a 5′ribozyme.

12. The method of claim 11, wherein the 3′ribozyme catalyzes itself out of the first RNA molecule, thereby generating a 3′P or 2′3′ cP end.

13. The method of any of claims 11-12, wherein the 5′ribozyme catalyzes itself out of the second RNA molecule, thereby generating a 5′OH end.

14. The method of claim 13, wherein the 3′P or 2′3′ cP end is ligated to the 5′OH end to form an RNA molecule comprising the coding region of the first RNA molecule and the coding region of the second RNA molecule.

15. The method of any of claims 11-14 wherein the 3′ ribozyme is a member of the HDV family of ribozymes.

16. The method of any of claims 11-14 wherein the 5′ ribozyme is a member of the HH family of ribozymes.

17. The method of any of claims 11-16, wherein the method further comprises administering to the cell or tissue one or more additional nucleic acid molecules encoding one or more additional RNA molecules, each additional RNA molecule comprising a coding region encoding a domain of the protein of interest; a 5′ ribozyme; and a 3′ ribozyme.

18. The method of any of claims 11-16, wherein the method further comprises administering to the cell or tissue one or more additional nucleic acid molecules encoding one or more additional RNA molecules, each additional RNA molecule comprising a coding region encoding a domain of the protein of interest; a 5′ ribozyme; and a 3′ ribozyme recognition sequence.

19. The method of claim 18, wherein the method further comprises administering to the cell or tissue a ribozyme that interacts with the 3′ ribozyme recognition sequence which induces the removal of the 3′ recognition sequence.

20. The method of claim 19, wherein the 3′ ribozyme recognition sequence comprises VS-S and wherein the ribozyme is VS-Rz.

21. The method of any of claim 11-20, wherein, the method further comprises administering to the cell or tissue a ligase to induce the assembly of the RNA molecule.

22. The method of claim 20, wherein the ligase is RNA 2′,3′-Cyclic Phosphate and 5′-OH (RtcB) ligase.

23. An in vitro method of generating an RNA molecule encoding a protein of interest comprising:
providing a first RNA molecule comprising a coding region encoding a first portion of the protein of interest and a 3′ribozyme;
providing a second RNA molecule comprising a coding region encoding a second portion of the protein of interest and a 5′ribozyme; and
providing a ligase to induce the assembly of the RNA molecule from the coding region of the first RNA molecule and the coding region of the second RNA molecule.

24. An in vitro method of generating an RNA molecule encoding a repeat domain protein of interest comprising:
a) providing a first RNA molecule comprising a coding region encoding a first portion of the protein of interest and a 3′ribozyme;
b) providing one or more additional RNA molecule comprising a coding region encoding a domain of the protein of interest, a 5′ ribozyme, and a 3′ ribozyme recognition sequence;
c) providing a ligase to ligate the coding region of the first RNA molecule and the coding region of the one or more additional RNA molecule;
d) providing a ribozyme that recognizes the 3′ ribozyme recognition sequence and catalyzes its removal;
e) repeating steps b)-d) one or more times to generate an RNA molecule encoding a plurality of repeat domains;
f) providing a last RNA molecule comprising a coding region encoding a last portion of the protein of interest and a 5′ribozyme; and
g) providing a ligase to ligate the coding region of the one or more additional RNA molecule and the coding region of the last RNA molecule, thereby generating a complete RNA molecule encoding a repeat domain protein.

25. A method of treating a disease or disorder in a subject caused by a mutation in a large protein of interest comprising:
administering to said subject a first nucleic acid molecule comprising a coding region encoding a first portion of the protein of interest and a 3′ribozyme; and
administering to said subject a second nucleic acid comprising a coding region encoding a second portion of the protein of interest and a 5′ribozyme.

26. The method of claim 25, wherein the disease or disorder is one or more selected from the group consisting of: Duchenne Muscular Dystrophy, autosomal recessive polycystic kidney disease, Hemophilia A, Stargardt macular degeneration, limb-girdle muscular dystrophies , DFNB9, neurosensory nonsyndromic recessive deafness, Cystic Fibrosis, Wilson Disease, Miyoshi Muscular Dystrophy and Deafness, Autosomal Recessive 9, Usher Syndrome, Type I and Deafness, Autosomal Recessive 2, Deafness, Autosomal Recessive 3 and Nonsyndromic Hearing Loss, Usher syndrome type I, autosomal recessive deafness-16 (DFNB16), Meniere's disease (MD), Deafness, Autosomal Dominant 12 and Deafness, Autosomal Recessive 21, Usher syndrome Type 1F (USH1F) and DFNB23, Deafness, Autosomal Recessive 28 and Nonsyndromic Hearing Loss, Deafness, Autosomal Recessive 30 and Nonsyndromic Hearing Loss, Otospondylomegaepiphyseal Dysplasia, Autosomal Recessive and Otospondylomegaepiphyseal Dysplasia, Autosomal Dominant, Deafness, Autosomal Recessive 77 and Autosomal Recessive Non-Syndromic Sensorineural Deafness Type Dfnb, autosomal-recessive nonsyndromic hearing impairment DFNB84, Deafness, Autosomal Recessive 84B and Rare Genetic Deafness, Peripheral Neuropathy, Myopathy, Hoarseness, And Hearing Loss and Deafness, Autosomal Dominant 4A, congenital thrombocytopenia, sensory hearing loss, DFNA56, HXB, deafness, autosomal dominant 56, hexabrachion , epileptic encephalopathy, Timothy Syndrome and Long Qt Syndrome8, X-linked retinal disorder, Hyperaldosteronism, Spinocerebellar Ataxia 42, Primary Aldosteronism, Seizures, And Neurologic Abnormalities and Sinoatrial Node Dysfunction And Deafness, Neurodevelopmental Disorder, hypokalemic periodic paralysis, Epilepsy, developmental and epileptic encephalopathies, Brody myopathy, Darier's disease/ Heart disease, von Willebrand disease, and Zellweger syndrome.

27. A system for generating an RNA molecule encoding a protein of interest and a circular RNA molecule comprising a nucleic acid encoding:
a first portion of a protein of interest;
a synthetic intron comprising a 5′ ribozyme, a cargo sequence, and a 3′ ribozyme; and
a second portion of a protein of interest.

28. The system of claim 27, wherein the protein of interest is one or more selected from the group consisting of: a therapeutic protein, a reporter protein, and a Cas9 protein.

29. The system of claim 27, wherein the cargo sequence is one or more selected from the group consisting of: a sequence encoding a therapeutic protein of interest, a CRISPR guide RNA sequence, a small RNA sequence, and a trans-cleaving ribozyme sequence. In one embodiment, said small RNA sequence comprises one or more selected from the group consisting of: microRNA (miRNA), Piwi-interacting RNA (piRNA), small interfering RNA (siRNA), small nucleolar RNA (snoRNAs), small tRNA-derived RNA (tsRNA), small rDNA-derived RNA (srRNA) and small nuclear RNA (snRNA).

30. The system of claim 27, wherein the 3′ ribozyme of the synthetic intron is a member of the HH family of ribozymes.

31. The system of claim 27, wherein the 5′ ribozyme of the synthetic intron is one or more selected from the group consisting of: a member of the HDV family of ribozymes, a member of the HDV family of ribozymes, and VS-S ribozyme recognition sequence.

32. The system of claim 27, further comprising one or more selected from the group consisting of: RtcB ligase and a nucleic acid encoding RtcB ligase.

33. A method of delivering an RNA molecule encoding a protein of interest and a circular RNA molecule, the method comprising:
administering to a cell or tissue a nucleic acid encoding a first portion of a protein of interest, a synthetic intron comprising a cis-cleaving 5′ ribozyme, a cargo sequence and a cis-cleaving 3′ ribozyme, and a second portion of a protein of interest.

34. The method of claim 33, wherein the protein of interest is one or more selected from the group consisting of: a therapeutic protein, a reporter protein, and a Cas9 protein.

35. The method of claim 33, wherein the cargo sequence is one or more selected from the group consisting of: a sequence encoding a therapeutic protein of interest, a CRISPR guide RNA sequence, a small RNA sequence, and a trans-cleaving ribozyme sequence. In one embodiment, said small RNA sequence comprises one or more selected from the group consisting of: microRNA (miRNA), Piwi-interacting RNA (piRNA), small interfering RNA (siRNA), small nucleolar RNA (snoRNAs), small tRNA-derived RNA (tsRNA), small rDNA-derived RNA (srRNA) and small nuclear RNA (snRNA).

36. The method of claim 33, further comprising administering to the cell or tissue one or more selected from the group consisting of: RtcB ligase and a nucleic acid encoding RtcB ligase.


SEQUENCE LISTING (130 sequences)

SEQ ID NO: 1 - RNA, 302 nt, Artificial Sequence
  Nt-GFP
auggugagcaagggcgaggagcuguucaccgggguggugcccauccuggucgagcuggacggcgacguaaacggccacaaguucagcguguccggcgagggcgagggcgaugccaccuacggcaagcugacccugaaguucaucugcaccaccggcaagcugcccgugcccuggcccacccucgugaccacccugaccuacggcgugcagugcuucagccgcuaccccgaccacaugaagcagcacgacuucuucaaguccgccaugcccgaaggcuacguccaggagcgcaccaucuucuu

SEQ ID NO: 2 - RNA, 421 nt, Artificial Sequence
  Ct-GFP
caaggacgacggcaacuacaagacccgcgccgaggugaaguucgagggcgacacccuggugaaccgcaucgagcugaagggcaucgacuucaaggaggacggcaacauccuggggcacaagcuggaguacaacuacaacagccacaacgucuauaucauggccgacaagcagaagaacggcaucaaggugaacuucaagauccgccacaacaucgaggacggcagcgugcagcucgccgaccacuaccagcagaacacccccaucggcgacggccccgugcugcugcccgacaaccacuaccugagcacccaguccgcccugagcaaagaccccaacgagaagcgcgaucacaugguccugcuggaguucgugaccgccgccgggaucacucucggcauggacgagcuguacaaguaguaa

SEQ ID NO: 3 - RNA, 831 nt, Artificial Sequence
  Nt-Luciferase
auggaagacgccaaaaacauaaagaaaggcccggcgccauucuauccgcuggaagauggaaccgcuggagagcaacugcauaaggcuaugaagagauacgcccugguuccuggaacaauugcuuuuacagaugcacauaucgagguggacaucacuuacgcugaguacuucgaaauguccguucgguuggcagaagcuaugaaacgauaugggcugaauacaaaucacagaaucgucguaugcagugaaaacucucuucaauucuuuaugccgguguugggcgcguuauuuaucggaguugcaguugcgcccgcgaacgacauuuauaaugaacgugaauugcucaacaguaugggcauuucgcagccuaccgugguguucguuuccaaaaagggguugcaaaaaauuuugaacgugcaaaaaaagcucccaaucauccaaaaaauuauuaucauggauucuaaaacggauuaccagggauuucagucgauguacacguucgucacaucucaucuaccucccgguuuuaaugaauacgauuuugugccagaguccuucgauagggacaagacaauugcacugaucaugaacuccucuggaucuacuggucugccuaaaggugucgcucugccucauagaacugccugcgugagauucucgcaugccagagauccuauuuuuggcaaucaaaucauuccggauacugcgauuuuaaguguuguuccauuccaucacgguuuuggaauguuuacuacacucggauauuugauauguggauuucgagucgucuuaauguauagauuugaagaagagcuguuucugaggagccuu

SEQ ID NO: 4 - RNA, 825 nt, Artificial Sequence
  Ct-Luciferase
caggauuacaagauucaaagugcgcugcuggugccaacccuauucuccuucuucgccaaaagcacucugauugacaaauacgauuuaucuaauuuacacgaaauugcuucugguggcgcuccccucucuaaggaagucggggaagcgguugccaagagguuccaucugccagguaucaggcaaggauaugggcucacugagacuacaucagcuauucugauuacacccgagggggaugauaaaccgggcgcggucgguaaaguuguuccauuuuuugaagcgaagguuguggaucuggauaccgggaaaacgcugggcguuaaucaaagaggcgaacugugugugagagguccuaugauuauguccgguuauguaaacaauccggaagcgaccaacgccuugauugacaaggauggauggcuacauucuggagacauagcuuacugggacgaagacgaacacuucuucaucguugaccgccugaagucucugauuaaguacaaaggcuaucagguggcucccgcugaauuggaauccaucuugcuccaacaccccaacaucuucgacgcaggugucgcaggucuucccgacgaugacgccggugaacuucccgccgccguuguuguuuuggagcacggaaagacgaugacggaaaaagagaucguggauuacgucgccagucaaguaacaaccgcgaaaaaguugcgcggaggaguuguguuuguggacgaaguaccgaaaggucuuaccggaaaacucgacgcaagaaaaaucagagagauccucauaaaggccaagaagggcggaaagaucgccguguaguaa

SEQ ID NO: 5 - DNA, 543 bp, Artificial Sequence
  N1L
atgggtcaggccaatacgccctggagcagtaaggcaaacgcggatgcctttataaattcattcatcagtgcagcatccaatactggttccttctctcaagaccaaatggaggacatgtcactcatcggcaatactctgatggctgccatggacaatatgggaggccgcataacaccatctaagttgcaggcgttggatatggccttcgcatcatcagtggccgagatcgcggctagtgagggcggcgacttgggagtcactaccaacgcgatcgcggatgccctcacttctgctttttatcaaacgaccggggttgtcaattcacgattcatatctgagatcaggagcctcataggaatgttcgcgcaggcttccgcaaatgacgtttatgcatctgctggctctggcagcgggggtggtgggtatggagccagctcagcatctgcggcttctgcaagtgctgctgccccgagtggcgtagcttatcaggctcctgctcaggctcaaatcagttttacgttgcgagggcaacaacctgtttcc

SEQ ID NO: 6 - DNA, 132 bp, Artificial Sequence
  AQ
ggtccttatggacccggtgctagcgctgcggcagcagccgctggcggttatggcccaggttcagggcaacaggggcctgggcaacaaggacctggccaacaaggtcctggtcagcagggtccagggcagcag

SEQ ID NO: 7 - DNA, 450 bp, Artificial Sequence
  NR3
ggcgctgcttccgctgcagtatcagtaggtggctatggacctcaatctagtagcgcccctgttgcctctgccgccgcatctcgactttcaagtcccgccgctagttccagggtcagttccgcggtatctagcttggtaagtagcggacccactaatcaagcggcactttcaaacacaatatcctcagtagtcagtcaagtaagcgcatcaaaccctggcttgtcagggtgtgacgttctggttcaggcacttctggaagttgtctcagcgttggtaagcatcctgggtagctcctccataggtcaaattaattatggcgcgagcgcccaatacacacaaatggtgggtcagagtgtggcgcaggcactcgcaggcgactacaaggatcatgacggagactataaggatcatgatatagattacaaggacgatgatgacaaggcctagtaa

SEQ ID NO: 8 - RNA, 453 nt, Artificial Sequence
  Nt-4xMTS
augaguguguugacgccguugcuucugcgagggcuuaccgggucugcuagaagacuuccgguccccagggccaagauacauagccucggagacccgaugucugugcucacuccucugcuuuugcgaggacugacuggguccgccagacgacucccggugccgagagcuaaaauccauagccugggaaaauuggcaacuaugucaguccugacgccgcuucuucuccggggucuuacagggucugcaagaaggcugccuguaccucgggcgaaaauucauagcuugggcgacccgaugaguguauugacgccccuguugcugagaggauugacugggucagcgcgccggcucccugucccccgagcuaagauucacucccuugguaagcugagaauccuccaaucaacgguuccgagagcaagagauccgccggucgccacgaggccucucgag

SEQ ID NO: 9 - RNA, 68 nt, Artificial Sequence
  HDV68
ggccggcauggucccagccuccucgcuggcgccggcugggcaacaugcuucggcauggcgaaugggac

SEQ ID NO: 10 - RNA, 67 nt, Artificial Sequence
  HDV67
gggucggcauggcaucuccaccuccucgcgguccgaccugggcuacuucgguaggcuaagggagaag

SEQ ID NO: 11 - RNA, 56 nt, Artificial Sequence
  HDV56
gagggauaguacagagccuccccguggcucccuuggauaaccaacugauacuguac

SEQ ID NO: 12 - RNA, 87 nt, Artificial Sequence
  Genomic HDV
ggccggcauggucccagccuccucgcuggcgccggcugggcaacauuccgaggggaccguccccucgguaauggcgaaugggaccca

SEQ ID NO: 13 - RNA, 91 nt, Artificial Sequence
  Antigenomic HDV
gggucggcauggcaucuccaccuccucgcgguccgaccugggcauccgaaggaggacgcacguccacucggauggcuaagggagagccacu

SEQ ID NO: 14 - RNA, 144 nt, Artificial Sequence
  VS Ribozyme
gcgguaguaagcagggaacucaccuccaauuucaguacugaaauugucguagcaguugacuacuguuaugugauugguagaggcuaagugacgguauuggcguaagucaguauugcagcacagcacaagcccgcuugcgagaau

SEQ ID NO: 15 - RNA, 21 nt, Artificial Sequence
  VS-S
gaagggcgucgucgccccgag

SEQ ID NO: 16 - RNA, 144 nt, Artificial Sequence; identical to SEQ ID NO: 14
  VS-Rz

SEQ ID NO: 17 - RNA, 291 nt, Artificial Sequence
  Nt-DTA
auggaccccgacgacgugguggacagcagcaagagcuucgugauggagaacuucagcagcuaccacggcaccaagcccggcuacguggacagcauccagaagggcauccagaagcccaagagcggcacccagggcaacuacgacgacgacuggaagggcuucuacagcaccgacaacaaguacgacgcugccggcuacagcguggacaacgagaacccccugagcggcaaggccggcggcguggugaaggugaccuaccccggccugaccaaggugcuggcccugaaggug

SEQ ID NO: 18 - RNA, 297 nt, Artificial Sequence
  Ct-DTA
gacaaugccgagaccaucaagaaggagcugggccugagccugaccgagccccugauggagcaggugggcaccgaggaguucaucaagagauucggcgacggcgccagcagaguggugcugagccugcccuucgccgagggcagcagcagcguggaguacaucaacaacugggagcaggccaaggcccugagcguggagcuggagaucaacuucgagaccagaggcaagagaggccaggacgccauguacgaguacauggcccaggcuugcgccggcaacagagugagaagauaguaa

SEQ ID NO: 19 - RNA, 717 nt, Artificial Sequence
  GFPcdn (no start ATG codon)
guuagcaagggcgaggagcucuucaccggggucguccccauccucgucgagcucgacggcgacguaaacggccacaaguucagcgucuccggcgagggcgagggcgaugccaccuacggcaagcucacccugaaguucaucugcaccaccggcaagcugcccgugcccuggcccacccucgugaccacccugaccuacggcgugcagugcuucagccgcuaccccgaccacaugaagcagcacgacuucuucaaguccgccaugcccgaaggcuacguccaggagcgcaccaucuucuucaaggacgacggcaacuacaagacccgcgccgaggugaaguucgagggcgacacccuggugaaccgcaucgagcugaagggcaucgacuucaaggaggacggcaacauccuggggcacaagcuggaguacaacuacaacagccacaacgucuauaucauggccgacaagcagaagaacggcaucaaggugaacuucaagauccgccacaacaucgaggacggcagcgugcagcucgccgaccacuaccagcagaacacccccaucggcgacggccccgugcugcugcccgacaaccacuaccugagcacccaguccgcccugagcaaagaccccaacgagaagcgcgaucacaugguccugcuggaguucgugaccgccgccgggaucacucucggcauggacgagcuguacaaguag

SEQ ID NO: 20 - RNA, 131 nt, Artificial Sequence
  F2-Myr
auggguuguuguuucagcaagacagcggcgaaaggugaagcagcagcagaaagaccaggcgaggcugcgguagcaucaagucccuccaaggcuaaugggcaggaaaacggacacgucaaaguuggaagcgu

SEQ ID NO: 21 - RNA, 685 nt, Artificial Sequence
  F2-RFP
agccaucaucaaggaguucaugcgcuucaaggugcacauggagggcuccgugaacggccacgaguucgagaucgagggcgagggcgagggccgccccuacgagggcacccagaccgccaagcugaaggugaccaaggguggcccccugcccuucgccugggacauccuguccccucaguucauguacggcuccaaggccuacgugaagcaccccgccgacauccccgacuacuugaagcuguccuuccccgagggcuucaagugggagcgcgugaugaacuucgaggacggcggcguggugaccgugacccaggacuccucccugcaggacggcgaguucaucuacaaggugaagcugcgcggcaccaacuuccccuccgacggccccguaaugcagaagaagaccaugggcugggaggccuccuccgagcggauguaccccgaggacggcgcccugaagggcgagaucaagcagaggcugaagcugaaggacggcggccacuacgacgcugaggucaagaccaccuacaaggccaagaagcccgugcagcugcccggcgccuacaacgucaacaucaaguuggacaucaccucccacaacgaggacuacaccaucguggaacaguacgaacgcgccgagggccgccacuccaccggcggcauggacgagcuguacaaguaguaa

SEQ ID NO: 22 - RNA, 2337 nt, Artificial Sequence
  Nt-uDys
augcuuuggugggaagaaguagaggacuguuaugaaagagaagauguucaaaagaaaacauucacaaaauggguaaaugcacaauuuucuaaguuugggaagcagcauauugagaaccucuucagugaccuacaggaugggaggcgccuccuagaccuccucgaaggccugacagggcaaaaacugccaaaagaaaaaggauccacaagaguucaugcccugaacaaugucaacaaggcacugcggguuuugcagaacaauaauguugauuuagugaauauuggaaguacugacaucguagauggaaaucauaaacugacucuugguuugauuuggaauauaauccuccacuggcaggucaaaaauguaaugaaaaauaucauggcuggauugcaacaaaccaacagugaaaagauucuccugagcuggguccgacaaucaacucguaauuauccacagguuaauguaaucaacuucaccaccagcuggucugauggccuggcuuugaaugcucucauccauagucauaggccagaccuauuugacuggaauagugugguuugccagcagucagccacacaacgacuggaacaugcauucaacaucgccagauaucaauuaggcauagagaaacuacucgauccugaagauguugauaccaccuauccagauaagaaguccaucuuaauguacaucacaucacucuuccaaguuuugccucaacaagugagcauugaagccauccaggaaguggaaauguugccaaggccaccuaaagugacuaaagaagaacauuuucaguuacaucaucaaaugcacuauucucaacagaucacggucagucuagcacagggauaugagagaacuucuuccccuaagccucgauucaagagcuaugccuacacacaggcugcuuaugucaccaccucugacccuacacggagcccauuuccuucacagcauuuggaagcuccugaagacaagucauuuggcaguucauugauggagagugaaguaaaccuggaccguuaucaaacagcuuuagaagaaguauuaucguggcuucuuucugcugaggacacauugcaagcacaaggagagauuucuaaugauguggaaguggugaaagaccaguuucauacucaugagggguacaugauggauuugacagcccaucagggccggguugguaauauucuacaauugggaaguaagcugauuggaacaggaaaauuaucagaagaugaagaaacugaaguacaagagcagaugaaucuccuaaauucaagaugggaaugccucaggguagcuagcauggaaaaacaaagcaauuuacauagaguuuuaauggaucuccagaaucagaaacugaaagaguugaaugacuggcuaacaaaaacagaagaaagaacaaggaaaauggaggaagagccucuuggaccugaucuugaagaccuaaaacgccaaguacaacaacauaaggugcuucaagaagaucuagaacaagaacaagucagggucaauucucucacucacaugguggugguaguugaugaaucuaguggagaucacgcaacugcugcuuuggaagaacaacuuaagguauugggagaucgaugggcaaacaucuguagauggacagaagaccgcuggguucuuuuacaagacauccuucucaaauggcaacgucuuacugaagaacagugccuuuuuagugcauggcuuucagaaaaagaagaugcagugaacaagauucacacaacuggcuuuaaagaucaaaaugaaauguuaucaagucuucaaaaacuggccguuuuaaaagcggaucuagaaaagaaaaagcaauccaugggcaaacuguauucacucaaacaagaucuucuuucaacacugaagaauaagucagugacccagaagacggaagcauggcuggauaacuuugcccgguguugggauaauuuaguccaaaaacuugaaaagaguacagcacagauuucacaggcugucaccaccacucagccaucacuaacacagacaacuguaauggaaacaguaacuacggugaccacaagggaacagauccugguaaagcaugcucaagaggaacuuccaccaccaccuccccaaaagaagaggcagauuacuguggaucuugaaagacuccaggaacuucaagaggccacggaugagcuggaccucaagcugcgccaagcugaggugaucaagggauccuggcagcccgugggcgaucuccucauugacucucuccaagaucaccucgagaaagucaaggcacuucgaggagaaauugcgccucugaaagagaacgugagccac

SEQ ID NO: 23 - RNA, 1974 nt, Artificial Sequence
  Ct-uDys-GFP
gucaaugaccuugcucgccagcuuaccacuuugggcauucagcucucaccguauaaccucagcacucuggaagaccugaacaccagauggaagcuucugcagguggccgucgaggaccgagucaggcagcugcaugaagcccacagggacuuugguccagcaucucagcacuuucuuuccacgucuguccagggucccugggagagagccaucucgccaaacaaagugcccuacuauaucaaccacgagacucaaacaacuugcugggaccaucccaaaaugacagagcucuaccagucuuuagcugaccugaauaaugucagauucucagcuuauaggacugccaugaaacuccgaagacugcagaaggcccuuugcuuggaucucuugagccugucagcugcaugugaugccuuggaccagcacaaccucaagcaaaaugaccagcccauggauauccugcagauuauuaauuguuugaccacuauuuaugaccgccuggagcaagagcacaacaauuuggucaacgucccucucugcguggauaugugucugaacuggcugcugaauguuuaugauacgggacgaacagggaggauccguguccugucuuuuaaaacuggcaucauuucccuguguaaagcacauuuggaagacaaguacagauaccuuuucaagcaaguggcaaguucaacaggauuuugugaccagcgcaggcugggccuccuucugcaugauucuauccaaauuccaagacaguugggugaaguugcauccuuugggggcaguaacauugagccaaguguccggagcugcuuccaauuugcuaauaauaagccagagaucgaagcggcccucuuccuagacuggaugagacuggaaccccaguccaugguguggcugcccguccugcacagaguggcugcugcagaaacugccaagcaucaggccaaauguaacaucugcaaagaguguccaaucauuggauucagguacaggagucuaaagcacuuuaauuaugacaucugccaaagcugcuuuuuuucuggucgaguugcaaaaggccauaaaaugcacuaucccaugguggaauauugcacuccgacuacaucaggagaagauguucgagacuuugccaagguacuaaaaaacaaauuucgaaccaaaagguauuuugcgaagcauccccgaaugggcuaccugccagugcagacugucuuagagggggacaacauggaaacugacacaauucuagaggugagcaagggcgaggagcuguucaccgggguggugcccauccuggucgagcuggacggcgacguaaacggccacaaguucagcguguccggcgagggcgagggcgaugccaccuacggcaagcugacccugaaguucaucugcaccaccggcaagcugcccgugcccuggcccacccucgugaccacccugaccuacggcgugcagugcuucagccgcuaccccgaccacaugaagcagcacgacuucuucaaguccgccaugcccgaaggcuacguccaggagcgcaccaucuucuucaaggacgacggcaacuacaagacccgcgccgaggugaaguucgagggcgacacccuggugaaccgcaucgagcugaagggcaucgacuucaaggaggacggcaacauccuggggcacaagcuggaguacaacuacaacagccacaacgucuauaucauggccgacaagcagaagaacggcaucaaggugaacuucaagauccgccacaacaucgaggacggcagcgugcagcucgccgaccacuaccagcagaacacccccaucggcgacggccccgugcugcugcccgacaaccacuaccugagcacccaguccgcccugagcaaagaccccaacgagaagcgcgaucacaugguccugcuggaguucgugaccgccgccgggaucacucucggcauggacgagcuguacaaguaa

SEQ ID NO: 24 - RNA, 68 nt, Artificial Sequence
  HDV68 catalytic mutant
ggccggcauggucccagccuccucgcuggcgccggcugggcaacaugcuucggcauggugaaugggac

SEQ ID NO: 25 - RNA, 56 nt, Artificial Sequence
  Hammerhead with stem 3 overhangs specific to Nt-Luc
gagccuuaccggauguguuuuccggucugaugaguccgguagcggacgaaaggcuc

SEQ ID NO: 26 - RNA, 54 nt, Artificial Sequence
  Twister with 5 nt P1 stem for Ct-Luc
agccuuaacacugccaaugccggucccaagcccggauaaaaguggagggaggcu

SEQ ID NO: 27 - RNA, 54 nt, Artificial Sequence
  Twister with 5 nt P1 stem for Ct-Luc and T6A mutation
agccuaaacacugccaaugccggucccaagcccggauaaaaguggagggaggcu

SEQ ID NO: 28 - RNA, 54 nt, Artificial Sequence
  Twister mutant with 5 nt P1 stem for Ct-Luc
agccuuaacucuuccaaugccggucccaagcccggauaaaaguggagggaggcu

SEQ ID NO: 29 - RNA, 54 nt, Artificial Sequence; identical to SEQ ID NO: 26
  Twister with 5 nt P1 stem for Ct-Luc

SEQ ID NO: 30 - RNA, 51 nt, Artificial Sequence
  Twister with 2 nt P1 stem for Ct-Luc
agccuuaacacugccaaugccggucccaagcccggauaaaaguggagggag

SEQ ID NO: 31 - RNA, 49 nt, Artificial Sequence
  Twister with 1 nt P1 stem for Ct-Luc
agccuuaacacugccaaugccggucccaagcccggauaaaaguggaggg

SEQ ID NO: 32 - RNA, 49 nt, Artificial Sequence; identical to SEQ ID NO: 31
  Twister with no P1 stem for Ct-Luc

SEQ ID NO: 33 - RNA, 53 nt, Artificial Sequence
  16HH stem 1 overhang specific to Ct-Luc
gaaucuuguaauccugcugaugaguccgugaggacgaaacgaguaagcucguc

SEQ ID NO: 34 - RNA, 51 nt, Artificial Sequence
  14HH stem 1 overhang specific to Ct-Luc
aucuuguaauccugcugaugaguccgugaggacgaaacgaguaagcucguc

SEQ ID NO: 35 - RNA, 49 nt, Artificial Sequence
  12HH stem 1 overhang specific to Ct-Luc
cuuguaauccugcugaugaguccgugaggacgaaacgaguaagcucguc

SEQ ID NO: 36 - RNA, 45 nt, Artificial Sequence
  8HH stem 1 overhang specific to Ct-Luc
uaauccugcugaugaguccgugaggacgaaacgaguaagcucguc

SEQ ID NO: 37 - RNA, 43 nt, Artificial Sequence
  6HH stem 1 overhang specific to Ct-Luc
auccugcugaugaguccgugaggacgaaacgaguaagcucguc

SEQ ID NO: 38 - RNA, 43 nt, Artificial Sequence
  6HH Mutant stem 1 overhang specific to Ct-Lu
auccugcugaugaguccgugaggacgagacgaguaagcucguc

SEQ ID NO: 39 - RNA, 41 nt, Artificial Sequence
  4HH stem 1 overhang specific to Ct-Luc
ccugcugaugaguccgugaggacgaaacgaguaagcucguc

SEQ ID NO: 40 - RNA, 55 nt, Artificial Sequence
  RzB stem1 overhang specific to Ct-Luc
uuguaauaauccugcugaugagucgcugggaugcgacgaaacgccuucgggcguc

SEQ ID NO: 41 - RNA, 52 nt, Artificial Sequence
  Splice Donor sequence for Nt vector
guaaguaucaagguuacaagacagguuuaaggagaccaauagaaacugggcu

SEQ ID NO: 42 - RNA, 81 nt, Artificial Sequence
  Splice Acceptor sequence for Ct vector
ugucgagacagagaagacucuugcguuucugauaggcaccuauuggucuuacugacauccacuuugccuuucucuccacag

SEQ ID NO: 43 - RNA, 560 nt, Artificial Sequence
  GCN4 5′ UTR uORFs
aaacaaaaacucacaacacagguuacucuccccccuaaauucaaauuuuuuuugcccaucaguuucacuagcgaauuauacaacucaccagccacacagcucacucaucuacuucgcaaucaaaacaaaauauuuuauuuuaguucaguuuauuaaguuauuaucaguaucguauuaaaaaauuaaagaucauugaaaaauggcuugcuaaaccgauuauauuuuguuuuuaaaguagauuauuauuagaaaauuauuaagagaauuauguguuaaauuuauugaaagagaaaauuuauuuucccuuauuaauuaaaguccuuuacuuuuuuugaaaacugucaguuuuuugaagaguuauuuguuuuguuaccaauugcuaucauguacccguagaauuuuauucaagauguuuccguaacgguuaccuuucugucaaauuauccagguuuacucgccaauaaaaauuucccuauacuaucauuaauuaaaucauuauuauuacuaaaguuuuguuuaccaauuugucugcucaagaaaauaaauuaaauacaaauaaa

SEQ ID NO: 44 - RNA, 148 nt, Artificial Sequence
  SRY 5′ UTR uORFs
guugaggggguguugagggcggagaaaugcaaguuucauuacaaaaguuaacguaacaaagaaucugguagaaaugaguuuuggauaguaaaauaaguuucgaacucuggcaccuuucaauuuugucgcacucuccuuguuuuugaca

SEQ ID NO: 45 - RNA, 343 nt, Artificial Sequence
  Hoxa9 TIE
gaaaaaacagaagagggaaggauaccagagcgguucauacagggcccagaaacuaggcgaggugaccccucagcaagacaaacaccucuugauguugacuggcgauuuuccccaucuccagucuggggagcgggacuaggcauacagaugauggagcuuagaacccgcuggcuagggaauaaaauucgcugggcaguuugugcucaaagaagugggccagggcgcuugugacacaaucagggcguuugugacacaaacccuugaggguuggcaguucucuccuuggcgguugcucugguugcucuguggggccuucccuguggagcaagggugaucuggccga

SEQ ID NO: 46 - RNA, 170 nt, Artificial Sequence
  Hoxa3 TIE
aggacaauucgucucuugggcugccgaagcgacagcugucagagaggcagaagcuucugggagccgcggucugaaggcuacgugugcugccuggucauucaaagugucaauuuuagguccagaaguguccaaaccacaaguucucaaaacucugaaaaauggcucccucc

SEQ ID NO: 47 - RNA, 38 nt, Artificial Sequence
  NRAS 5′UTR G-quadruplex
cgucccgugugggaggggcgggucugggugcggccugc

SEQ ID NO: 48 - RNA, 126 nt, Artificial Sequence
  Human IFNG 5′ UTR pseudoknot
cacauuguucugaucaucugaagaucagcuauuagaagagaaagaucaguuaaguccuuuggaccugaucagcuugauacaagaacuacugauuucaacuucuuuggcuuaauucucucggaaacg

SEQ ID NO: 49 - RNA, 132 nt, Artificial Sequence
  Rat ODC 5′UTR
ugucagucccugcagccgccgccgccggccgccuucagucagcagcucggcgccaccuccggucggcgacugcggcgggcucgacgaggcggcugacggggcggcggcgggaagacggccgggugcgccuug

SEQ ID NO: 50 - RNA, 49 nt, Artificial Sequence
  SIRLOIN RNA Nuclear Localization Signal
cgccucccggguucaagcgauucuccugccucagccucccgaguagcug

SEQ ID NO: 51 - RNA, 42 nt, Artificial Sequence
  BORG lncRNA NLS
accucagaaucuacaagucagccccaauuaaauguuguuuua

SEQ ID NO: 52 - protein, 108 aa, Artificial Sequence
  FKBP DD
MGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKPE

SEQ ID NO: 53 - protein, 40 aa, Artificial Sequence
  PEST (enhanced ODC PEST)
SHGFPPEVEEQAAGTLPMSCAQESGMDRHPAACASARINV

SEQ ID NO: 54 - protein, 40 aa, Artificial Sequence
  ODC PEST (yeast)
SHGFPPEVEEQDDGTLPMSCAQESGMDRHPAACASARINV

SEQ ID NO: 55 - protein, 40 aa, Artificial Sequence
  ODC PEST (human)
NPDFPPEVEEQDASTLPVSCAWESGMKRHRAACASASINV

SEQ ID NO: 56 - protein, 57 aa, Artificial Sequence
  CL1
ACKNWFSSLSHFVIHLNSHGFPPEVEEQAAGTLPMSCAQESGMDRHPAACASARINV

SEQ ID NO: 57 - protein, 57 aa, Artificial Sequence; identical to SEQ ID NO: 56
  CL1-PEST

SEQ ID NO: 58 - protein, 68 aa, Artificial Sequence
  E1A PEST
SRECNSSTDSCDSGPSNTPPEIHPVVPLCPIKPVAVRVGGRRQAVECIEDLLNEPGQPLDLSCKRPRP

SEQ ID NO: 59 - protein, 31 aa, Artificial Sequence
  C-myc PEST
LHEETPPTTSSDSEEEQEDEEEIDVVSVEKR

SEQ ID NO: 60 - protein, 25 aa, Artificial Sequence
  c-Fos PEST
AAHRKGSSSNEPSSDSLSSPTLLAL

SEQ ID NO: 61 - protein, 26 aa, Artificial Sequence
  v-Myb PEST
PSPPVDHGCLPEESASPARCMIVHQS

SEQ ID NO: 62 - protein, 59 aa, Artificial Sequence
  NPDC1 PEST
PPKELDTASSDEENEDGDFTVYECPGLAPTGEMEVRNPLFDHAALSAPLPAPSSPPALP

SEQ ID NO: 63 - protein, 37 aa, Artificial Sequence
  IkBa PEST
PESEDEESYDTESEFTEFTEDELPYDDCVFGGQRLTL

SEQ ID NO: 64 - protein, 41 aa, Artificial Sequence
  m.m. AZIN2 PEST
GQLLPAEEDQDAEGVCKPLSCGWEITDTLCVGPVFTPASIM

SEQ ID NO: 65 - protein, 43 aa, Artificial Sequence
  x.l. AZIN2 PEST
VQLLQRGLQQTEEKENVCTPMSCGWEISDSLCFTRTFAATSII

SEQ ID NO: 66 - protein, 12 aa, Artificial Sequence
  NS1
TSLYKKVGMGRK

SEQ ID NO: 67 - protein, 12 aa, Artificial Sequence
  NS6
SLYKKVGTMAAG

SEQ ID NO: 68 - protein, 12 aa, Artificial Sequence
  NS7
YKKVGTMRGRGL

SEQ ID NO: 69 - protein, 12 aa, Artificial Sequence
  NS12
ERAPTGRWGRRG

SEQ ID NO: 70 - protein, 12 aa, Artificial Sequence
  NS15
EGPLWHPRICGS

SEQ ID NO: 71 - protein, 12 aa, Artificial Sequence
  SELK
LRGPSPPPMAGG

SEQ ID NO: 72 - protein, 12 aa, Artificial Sequence
  SELS
WRPGRRGPSSGG

SEQ ID NO: 73 - protein, 5 aa, Artificial Sequence
  EMID1
RDERG

SEQ ID NO: 74 - protein, 5 aa, Artificial Sequence
  IRX6
GAEAG

SEQ ID NO: 75 - protein, 80 aa, Artificial Sequence
  UbVR
QIFVKTLTGKTITLEVEPSDTIENVKAKIQDKEGIPPDQQRLIFAGKQLEDGRTLSDYNIQKESTLHLVLRLRGVRASAS

SEQ ID NO: 76 - protein, 162 aa, Artificial Sequence
  2xUbVR
TSQIFVKTLTGKTITLEVEPSDTIENVKAKIQDKEGIPPDQQRLIFAGKQLEDGRTLSDYNIQKESTLHLVLRLRGVRASASQIFVKTLTGKTITLEVEPSDTIENVKAKIQDKEGIPPDQQRLIFAGKQLEDGRTLSDYNIQKESTLHLVLRLRGVRASAS

SEQ ID NO: 77 - DNA, 39 bp, Artificial Sequence
  12x poly K encoding tail sequence
aaaaaaaaaaaaaaaaaaaaaaaaaaaaaaaaaaaataa

SEQ ID NO: 78 - protein, 12 aa, Artificial Sequence
  Translation Product 12x poly K
KKKKKKKKKKKK

SEQ ID NO: 79 - DNA, 51 bp, Artificial Sequence
  16x poly K encoding tail sequence
aaaaaaaaaaaaaaaaaaaaaaaaaaaaaaaaaaaaaaaaaaaaaaaataa

SEQ ID NO: 80 - protein, 16 aa, Artificial Sequence
  Translation Product 16x poly K
KKKKKKKKKKKKKKKK

SEQ ID NO: 81 - protein, 505 aa, Artificial Sequence
  Human RtcB protein sequence
MSRSYNDELQFLEKINKNCWRIKKGFVPNMQVEGVFYVNDALEKLMFEELRNACRGGGVGGFLPAMKQIGNVAALPGIVHRSIGLPDVHSGYGFAIGNMAAFDMNDPEAVVSPGGVGFDINCGVRLLRTNLDESDVQPVKEQLAQAMFDHIPVGVGSKGVIPMNAKDLEEALEMGVDWSLREGYAWAEDKEHCEEYGRMLQADPNKVSARAKKRGLPQLGTLGAGNHYAEIQVVDEIFNEYAAKKMGIDHKGQVCVMIHSGSRGLGHQVATDALVAMEKAMKRDKIIVNDRQLACARIASPEGQDYLKGMAAAGNYAWVNRSSMTFLTRQAFAKVFNTTPDDLDLHVIYDVSHNIAKVEQHVVDGKERTLLVHRKGSTRAFPPHHPLIAVDYQLTGQPVLIGGTMGTCSYVLTGTEQGMTETFGTTCHGAGRALSRAKSRRNLDFQDVLDKLADMGIAIRVASPKLVMEEAPESYKNVTDVVNTCHDAGISKKAIKLRPIAVIKG

SEQ ID NO: 82 - DNA, 1518 bp, Artificial Sequence
  Human RtcB human codon optimized nucleic acid sequence
atgtcccggtcatataatgacgagctgcaattccttgagaagataaataagaattgctggcgcatcaagaaaggcttcgttcctaatatgcaagttgaaggtgtattttatgtaaatgacgctttggaaaagttgatgttcgaggaactgaggaacgcatgtcgcggtggaggtgtcgggggttttcttcccgctatgaagcagattggcaatgtggcggctctgcccggaattgtgcaccgctctataggattgcctgacgtacacagcggctacggattcgccattgggaatatggcggcgttcgatatgaacgaccctgaggcggttgttagccctggaggtgtcggcttcgatataaattgcggagtcagattgcttcggacaaatttggatgaatctgacgtacaaccagtgaaagagcaacttgcacaagcgatgttcgatcatattcccgtgggtgtggggtcaaagggagtaatcccaatgaacgcgaaagacctggaagaagcattggagatgggtgtagactggtcactgcgagaaggttatgcctgggctgaagacaaagagcactgcgaggagtacggtcgcatgttgcaagcagacccaaataaagtatccgcgagggccaagaaaagaggtttgccgcagctggggacattgggggccggtaaccactatgcagaaatacaagtagtggatgagattttcaatgagtacgctgcgaagaaaatggggatcgaccataaaggtcaagtgtgcgtaatgatacattctgggagtcgcggactcgggcaccaagttgcaacggacgcccttgtcgccatggaaaaagcgatgaagcgggataaaatcatcgtaaatgataggcaattggcttgcgctcgcattgcgagtccggaagggcaagactacttgaaagggatggctgctgccgggaattatgcatgggtcaaccggagcagtatgacattcttgacgcggcaggcttttgcaaaagtgtttaatacgactccggacgacctcgatctccatgttatatatgatgtatcacacaatatcgcaaaggttgagcaacacgttgtggatggtaaggaaaggactctgctggtacaccggaaaggcagtacacgggcattcccgcctcatcacccattgatcgcagtcgattatcaattgacaggtcagccagttctgatcggaggaacaatgggcacatgtagctacgtattgaccgggactgaacaggggatgaccgaaacttttggcacaacatgccatggcgcggggagggcactctcccgagctaaaagtaggaggaatcttgacttccaggatgtactggataagctggccgatatggggatagccatccgggtagcgtcacccaaattggtaatggaggaagctcctgaaagctataaaaatgtcactgacgttgtcaacacatgccatgacgcgggtatatccaagaaagctattaagctgcgcccaatagctgtaattaaaggatag

SEQ ID NO: 83 - protein, 408 aa, Artificial Sequence
  E. Coli RtcB protein sequence
MNYELLTTENAPVKMWTKGVPVEADARQQLINTAKMPFIFKHIAVMPDVHLGKGSTIGSVIPTKGAIIPAAVGVDIGCGMNALRTALTAEDLPENLAELRQAIETAVPHGRTTGRCKRDKGAWENPPVNVDAKWAELEAGYQWLTQKYPRFLNTNNYKHLGTLGTGNHFIEICLDESDQVWIMLHSGSRGIGNAIGTYFIDLAQKEMQETLETLPSRDLAYFMEGTEYFDDYLKAVAWAQLFASLNRDAMMENVVTALQSITQKTVRQPQTLAMEEINCHHNYVQKEQHFGEEIYVTRKGAVSARAGQYGIIPGSMGAKSFIVRGLGNEESFCSCSHGAGRVMSRTKAKKLFSVEDQIRATAHVECRKDAEVIDEIPMAYKDIDAVMAAQSDLVEVIYTLRQVVCVKG

SEQ ID NO: 84 - DNA, 1227 bp, Artificial Sequence
  E. Coli RtcB human codon optimized nucleic acid sequence
atgaattacgagcttcttaccactgagaatgcacctgtgaaaatgtggactaagggagtgcccgtggaagcggacgcaaggcagcagctcataaatacagctaagatgcctttcatcttcaaacacatcgcggttatgcccgacgtgcacctcggaaaaggctctactattggaagtgtgattccgacaaagggtgcgatcatacctgctgccgtcggggtggacataggctgtggaatgaatgccctgcgaacggctcttaccgcagaagatcttcctgagaatctggccgagctgcgacaggccattgaaacagcggttccgcatggtcggactaccggacggtgcaaaagggacaaaggtgcgtgggaaaaccctcccgttaacgtggatgcgaaatgggctgagttggaagcaggctatcaatggcttacccagaaatatccacggttcttgaacactaataactacaaacacctggggaccttggggacggggaatcatttcatcgaaatctgtcttgatgagtctgaccaagtgtggattatgcttcatagcggtagccgcggcattggtaacgcaattgggacatattttattgacctcgcgcagaaagagatgcaggaaacgcttgagacgctgccgtcccgagatcttgcgtattttatggaagggacggaatactttgacgattatctgaaggcggtagcatgggctcaactgtttgctagtctcaaccgagacgcgatgatggaaaatgtggtaacagcacttcaatcaatcacccaaaagacagtgcgacagccccaaactctcgctatggaagaaatcaattgccaccacaattacgttcagaaagagcaacatttcggagaagaaatttacgtgacaagaaaaggagctgttagcgcgagggccggacagtacggcatcattcctgggtcaatgggtgcgaaatcttttatagtacgcgggcttggtaatgaagaatccttctgcagctgttctcatggagccggaagggtaatgtccaggactaaggccaagaaactcttctctgtggaagatcaaattagagctacagcacatgttgaatgtagaaaggatgccgaagtcatagacgagatccctatggcttacaaagatatagatgctgtaatggctgcacagtcagacctcgtagaggttatctacacactccggcaagtcgtatgcgtaaaaggatag

SEQ ID NO: 85 - protein, 470 aa, Artificial Sequence
  Deinococcus radiodurans RtcB protein sequence
MNGKHITKLGFEGKAVGLALSAAGLREDAGVSRGDILDELRSVQNYPEQYQGGGVYADLATHLIEQQAAQQTRQSAKLRAAPLPYRTWGEDLIEPGAHRQMDVAMQLPISRAGALMPDAHVGYGLPIGGVLATENAVIPYGVGVDIGCSMMLSVFPVAATGLSVDEARSLLLKHTRFGAGVGFEKRDRLDHPVLAEATWDEQPLLRHLFDKAAGQIGSSGSGNHFVEFGTFTLAQADPQLEGLDPGEYLAVLSHSGSRGFGAQVAGHFTNLAQRLWPALDKEAQKLAWLPLDSEAGQAYWQAMNLAGRYALANHEQIHARLARALGEKPLLRAQNSHNLAWKQQVNGQELIVHRKGATPAEAGQLGLIPGSMADPGYLVRGRGNPEALASASHGAGRQLGRKAAERSLAKKDVQAYLKDRGVTLIGGGIDEAPQAYKRIEDVIARQRDLVDVLGEFRPRVVRMDTGSEDV

SEQ ID NO: 86 - DNA, 1413 bp, Artificial Sequence
  Deinococcus radiodurans RtcB human codon optimized nucleic acid sequence
atgaacggaaagcacatcacgaagttgggtttcgaagggaaggctgttggcctggcattgtctgcggctggtctcagggaagacgcaggcgtttcccgaggagatattctcgatgaacttaggtctgtccagaattatccggagcaatatcaagggggaggggtctatgccgacttggcgacacaccttattgagcaacaagctgctcagcagactaggcaatccgccaagctgcgagcagcaccacttccgtaccgaacgtggggtgaagacctgatcgagccaggcgcacacagacagatggatgtagcaatgcagctcccgatctcccgggcgggagcgctgatgccagatgcccacgtaggatacggacttcccattggaggcgtgctcgctaccgaaaacgccgtaatcccctatggagtgggcgttgacatcggttgctcaatgatgttgagtgttttcccggtggctgcaacaggtctgtcagtggatgaggcgcggtcactgcttctcaaacacacgcgcttcggtgcgggggtcggattcgagaaacgcgacaggctcgaccatcctgtcttggcggaggctacgtgggacgagcagcctttgctgagacacttgtttgataaagctgctggccagattgggtcttccggatcagggaaccacttcgtcgaatttggaactttcaccctcgcacaggccgatccgcagttggaaggtttggaccctggggaatacttggctgttctttcacactcagggagtagaggatttggagcccaggtggctgggcattttaccaacttggcgcagcgcttgtggcccgcacttgataaggaagctcaaaaactcgcatggctgccactggattctgaggctgggcaagcctactggcaagccatgaacttggcgggacgatatgcgttggctaaccatgagcaaattcacgcccgactggcccgcgcacttggtgagaagcctcttctgcgcgcccagaactcccacaatctggcctggaaacagcaggtgaatgggcaggaattgatagtccaccgcaaaggggctactcctgcggaagccgggcaacttggtctcatccctggctccatggccgacccgggatatttggtcaggggaaggggaaatccggaagcattggcctctgcgtcacacggagcaggtagacagctcggccggaaggcagcggaaaggtccctggcgaagaaagatgtgcaggcttaccttaaagatagaggagtaacccttatcgggggcgggattgacgaggctccccaggcgtataaaaggatcgaagacgtcatagcacgccagcgggaccttgtggatgtgttgggagaatttaggccacgagtagtgcggatggatacagggtctgaagatgtttag

SEQ ID NO: 87 - protein, 481 aa, Artificial Sequence
  Pyrococcus horikoshii RtcB protein sequence
MVVPLKRIDKIRWEIPKFDKRMRVPGRVYADEVLLEKMKNDRTLEQATNVAMLPGIYKYSIVMPDGHQGYGFPIGGVAAFDVKEGVISPGGIGYDINCGVRLIRTNLTEKEVRPRIKQLVDTLFKNVPSGVGSQGRIKLHWTQIDDVLVDGAKWAVDNGYGWERDLERLEEGGRMEGADPEAVSQRAKQRGAPQLGSLGSGNHFLEVQVVDKIFDPEVAKAYGLFEGQVVVMVHTGSRGLGHQVASDYLRIMERAIRKYRIPWPDRELVSVPFQSEEGQRYFSAMKAAANFAWANRQMITHWVRESFQEVFKQDPEGDLGMDIVYDVAHNIGKVEEHEVDGKRVKVIVHRKGATRAFPPGHEAVPRLYRDVGQPVLIPGSMGTASYILAGTEGAMKETFGSTCHGAGRVLSRKAATRQYRGDRIRQELLNRGIYVRAASMRVVAEEAPGAYKNVDNVVKVVSEAGIAKLVARMRPIGVAKG

SEQ ID NO: 88 - DNA, 1446 bp, Artificial Sequence
  Pyrococcus horikoshii RtcB human codon optimized nucleic acid sequence
atggtggttcccctgaagagaatagataaaattcgctgggagatccctaagttcgacaaaaggatgagagtaccaggacgggtgtatgcagatgaggtcttgctcgaaaaaatgaaaaatgaccgcacgcttgaacaggcaacgaacgtcgcaatgctgccaggcatttataaatacagtattgtgatgcccgatggccaccaggggtacggatttccaattggaggggtagccgctttcgatgttaaagagggcgtaatcagtcctggtgggatcgggtacgacatcaattgtggagtccgactgatcagaaccaatctcactgagaaagaagtaaggcccagaatcaagcaactggttgatactctgtttaaaaacgtcccttctggagtgggcagtcaagggcggattaaactgcattggactcaaatagacgatgtactcgtagacggggcaaaatgggctgtggacaacggatatggatgggagcgcgacctcgaacggttggaagaaggtggtcggatggagggggccgatccagaggcggtctcccaacgggcaaagcagaggggagcaccccagctcgggtccctggggtctggcaaccatttcctcgaagtacaggtcgtagataagatctttgatcctgaagtagcgaaagcgtatggcctcttcgaggggcaagtggttgtgatggttcacactggtagcagaggtcttgggcaccaagttgcatccgactacttgcgaatcatggagcgcgcaattaggaagtatagaatcccctggccggatagagagcttgtctcagtcccttttcaaagcgaggaaggacaaagatacttcagcgccatgaaagccgcggcaaactttgcatgggcaaatcggcagatgataactcattgggtacgagaatcattccaagaggtcttcaaacaagatccggaaggcgacctcggcatggacattgtgtacgatgtcgcccacaatataggcaaagtggaggagcacgaggtcgatggcaaacgggtgaaagttatagtccatcgaaagggagcaactcgcgcttttccaccaggtcacgaggctgtacctaggctgtatcgggatgtcggtcaacctgtactcatacccggatctatgggcacagcttcctatattctggctggcactgaaggagcaatgaaagagacgtttggatctacctgtcacggagctggtagggtactctcccggaaggccgcgacacgacaatatcgcggggacaggatcagacaagaacttttgaatagaggcatctacgtgcgcgccgctagtatgcgcgtcgtggccgaagaggcacctggggcttacaagaacgtggataacgtagttaaagtagtaagtgaagccggcatcgccaagctggtggcccggatgcgcccgattggcgtggcaaagggttag

SEQ ID NO: 89 - protein, 481 aa, Artificial Sequence
  Pyrococcus sp. ST04 RtcB protein sequence
MTVPLKRIDRIRWEIPKFDKRMRVPGRVYADEVLIEKMRSDRTLEQAANVAMLPGIYKYSIVMPDGHQGYGFPIGGVAAFDVKEGVISPGGIGYDINCGVRLIRTNLTEKEVRPKIKQLVDTLFKNVPSGVGSQGRIRLHWTQIDDVLVDGAKWAVDNGYGWERDLERLEEGGRMEGADPDAVSQRAKQRGAPQLGSLGSGNHFLEVQVVDKIYDEEVAKAYGLFEGQVVVMVHTGSRGLGHQVASDYLRIMERAIRKYRIPWPDRELVSVPFQSEEGQRYFSAMKAAANFAWANRQMITHWVRESFQEVFRQDPEGDLGMDIVYDVAHNIGKVEEHEVDGKKVTVIVHRKGATRAFPPGHEAIPRIYRDVGQPVLIPGSMGTASYVLAGTEGAMKETFGSTCHGAGRVLSRKAATRQYRGDRIRNELLQRGIYVRAASMRVVAEEAPGAYKNVDNVVKVVSEAGIAKLVARMRPIGVAKG

SEQ ID NO: 90 - DNA, 1446 bp, Artificial Sequence
  Pyrococcus sp. ST04 RtcB human codon optimized nucleic acid sequence
atgaccgttcccctgaagagaatagataggattcgctgggagatccctaagttcgacaaaaggatgagagtaccaggacgggtgtatgcagatgaggtcttgatcgagaaaatgagaagcgaccgcacgcttgaacaggcagccaacgtcgcaatgctgccaggcatttataaatacagtattgtgatgcccgatggccaccaggggtacggatttccaattggaggggtagccgctttcgatgttaaagagggcgtaatcagtcctggtgggatcgggtacgacatcaattgtggagtccgactgatcagaaccaatctcactgagaaagaagtaaggcccaaaatcaagcaactggttgatactctgtttaaaaacgtcccttctggagtgggcagtcaagggcggattagactgcattggactcaaatagacgatgtactcgtagacggggcaaaatgggctgtggacaacggatatggatgggagcgcgacctcgaacggttggaagaaggtggtcggatggagggggccgatccagacgcggtctcccaacgggcaaagcagaggggagcaccccagctcgggtccctggggtctggcaaccatttcctcgaagtacaggtcgtagataagatctacgatgaggaagtagcgaaagcgtatggcctcttcgaggggcaagtggttgtgatggttcacactggtagcagaggtcttgggcaccaagttgcatccgactacttgcgaatcatggagcgcgcaattaggaagtatagaatcccctggccggatagagagcttgtctcagtcccttttcaaagcgaggaaggacaaagatacttcagcgccatgaaagccgcggcaaactttgcatgggcaaatcggcagatgataactcattgggtacgagaatcattccaagaggtcttcagacaagatccggaaggcgacctcggcatggacattgtgtacgatgtcgcccacaatataggcaaagtggaggagcacgaggtcgatggcaagaaagtgaccgttatagtccatcgaaagggagcaactcgcgcttttccaccaggtcacgaggctatccctaggatctatcgggatgtcggtcaacctgtactcatacccggatctatgggcacagcttcctatgtgctggctggcactgaaggagcaatgaaagagacgtttggatctacctgtcacggagctggtagggtactctcccggaaggccgcgacacgacaatatcgcggggacaggatcagaaatgaacttttgcaaagaggcatctacgtgcgcgccgctagtatgcgcgtcgtggccgaagaggcacctggggcttacaagaacgtggataacgtagttaaagtagtaagtgaagccggcatcgccaagctggtggcccggatgcgcccgattggcgtggcaaagggttag

SEQ ID NO: 91 - protein, 480 aa, Artificial Sequence
  Thermococcus sp. EP1 RtcB protein sequence
MEIPLKRLDKIRWEIPKFNRRMRVPGRVYADDTLLQKMRQDKTLEQATNVAMLPGIYKYSIVMPDGHQGYGFPIGGVAAFDVKEGVISPGGVGYDINCGVRLIRTNLVEKEVRPKIKQLIDTLFKNVPSGLGSKGRIRLHWTQLDDVLADGAKWAVDNGYGWKDDLEHLEEGGRMEGANPNAVSQKAKQRGAPQLGSLGSGNHFLEIQVVDKVFNEEIAKAYGLFEGQIVVMVHTGSRGLGHQVASDYLRIMEKANRKYNVPWPDRELVSVPFQTEEGQRYFSAMKAAANFAWANRQMITHWVRESFEEVFKQKAEDLGMHIVYDVAHNIAKVEEHEVNGRKIKVVVHRKGATRAFPAGHEAIPKAYRDVGQPVLIPGSMGTASYVLAGAEGSMRETFGSTCHGAGRVLSRHAATRQFRGDRLRNELMQRGIYIRAASMRVVAEEAPGAYKNVDNVVRVVHEAGIANLVARMRPIGVAKG

SEQ ID NO: 92 - DNA, 1446 bp, Artificial Sequence
  Thermococcus sp. EP1 RtcB human codon optimized nucleic acid sequence
atggagataccactcaaacgacttgacaagatccgatgggagattcccaaatttaacagacgaatgagagttccgggaagagtttacgcagatgatacattgctccaaaagatgcgacaagataagacgctcgaacaagccaccaacgtggccatgctcccaggcatttataagtatagtatagtcatgcctgacggacaccagggttatggattcccgattggcggtgtagcagccttcgacgtaaaagagggagtaattagtcctggcggtgttggttatgatattaactgtggcgtgaggcttatcaggacgaatcttgtagagaaggaagtgcgaccaaaaatcaaacaacttatagatactttgttcaaaaatgtcccgtctgggctcggatcaaagggtcggataaggctccactggactcaactggatgatgttctggctgatggggcaaaatgggctgttgacaatgggtacgggtggaaggatgatctcgaacatttggaggagggcggacggatggagggcgcaaaccccaatgccgtttcacagaaagcgaagcaaaggggagcgccacagcttgggtcccttggctcaggcaatcatttcctcgaaattcaggtcgtcgataaggtttttaacgaagagatagcaaaggcttacggactctttgaaggtcagatagtggtaatggtccatacgggctctcggggactgggacatcaagtcgcaagtgactacctgaggatcatggagaaagccaatcgcaagtacaatgtgccctggcctgaccgggagcttgttagcgtgcccttccagacggaagagggtcaacgatactttagcgctatgaaggcggcagctaatttcgcttgggcaaacagacagatgataacacattgggttagagagtccttcgaggaggtctttaaacaaaaagctgaggaccttggaatgcatattgtctatgatgttgcccataacatagcaaaagtagaggaacatgaggtgaacgggcggaaaattaaggtcgtagtacacagaaaaggcgctaccagagcattccccgcaggacacgaggccatacccaaagcatatagagatgtcggccagccagtgctcataccgggatctatgggtacggcgtcctatgtcttggcgggtgctgaaggatcaatgagggagacgttcggctcaacctgtcatggggcaggtcgggtcttgtctcggcatgctgcaactcggcagttccgcggggatcgactcaggaatgaactcatgcagagaggcatttacatacgcgctgcctccatgcgcgttgtcgccgaggaagctcccggcgcctataagaacgtagacaatgtcgtcagggtggtgcatgaagcgggaattgcgaacttggtagccaggatgcgcccaataggggttgccaagggatagtaa

SEQ ID NO: 93 - protein, 167 aa, Artificial Sequence
  Human Archease protein sequence
MAQEEEDVRDYNLTEEQKAIKAKYPPVNRKYEYLDHTADVQLHAWGDTLEEAFEQCAMAMFGYMTDTGTVEPLQTVEVETQGDDLQSLLFHFLDEWLYKFSADEFFIPREVKVLSIDQRNFKLRSIGWGEEFSLSKHPQGTEVKAITYSAMQVYNEENPEVFVIIDI

SEQ ID NO: 94 - DNA, 461 bp, Artificial Sequence
  Human Archease human codon optimized nucleic acid sequence
aggaacaaaaggccatcaaagcgaaatatccgcctgtaaaccgaaagtatgagtacctggatcacactgcggacgtccagttgcatgcctggggcgacactctggaggaggcattcgaacaatgtgcaatggcaatgtttggctacatgactgatacaggcacagtggagccccttcaaacggtagaggtagaaactcagggagatgatcttcagagcttgctcttccattttctcgacgaatggttgtataagttcagtgccgacgagttcttcattccacgcgaagtgaaagtgctgagtattgatcagagaaactttaaacttaggtctattgggtggggtgaagagttctctttgtctaaacaccctcaaggaactgaggtaaaggcgataacttactcagccatgcaggtatataacgaggagaatcctgaggttttcgtaatcattgatatatag

SEQ ID NO: 95 - protein, 142 aa, Artificial Sequence
  Pyrococcus horikoshii Archease protein sequence
MKKWEHYEHTADIGIRGYGDSLEEAFEAVAIALFDVMVNVNKVEKKEVREIEVEAEDLEALLYSFLEELLVIHDIEGLVFRDFEVKIERVNGKYRLRAKAYGEKLDLKKHEPKEEVKAITYHDMKIERLPNGKWMAQLVPDI

SEQ ID NO: 96 - DNA, 429 bp, Artificial Sequence
  Pyrococcus horikoshii Archease human codon optimized nucleic acid sequence
atgaagaaatgggagcactatgagcatactgccgacattggtattcggggatatggggatagccttgaggaggcattcgaagcagtagccatcgcgctctttgatgtaatggtgaacgtgaataaagtcgagaagaaggaagtccgagaaattgaagtggaggcagaagatttggaggccctcctttattcattcctggaagaactgttggttattcatgatatagagggactggttttcagggactttgaagttaagatagagagagtaaatggcaaataccgacttcgagcgaaagcctacggtgagaagctcgacctcaagaagcacgaaccgaaagaggaagtaaaggcgataacctaccatgatatgaaaattgaacggttgcccaatggaaagtggatggctcaactcgttccagatatttag

SEQ ID NO: 97 - protein, 301 aa, Artificial Sequence
  T4 Polynucleotide Kinase (T4 PNK) protein sequence
MKKIILTIGCPGSGKSTWAREFIAKNPGFYNINRDDYRQSIMAHEERDEYKYTKKKEGIVTGMQFDTAKSILYGGDSVKGVIISDTNLNPERRLAWETFAKEYGWKVEHKVFDVPWTELVKRNSKRGTKAVPIDVLRSMYKSMREYLGLPVYNGTPGKPKAVIFDVDGTLAKMNGRGPYDLEKCDTDVINPMVVELSKMYALMGYQIVVVSGRESGTKEDPTKYYRMTRKWVEDIAGVPLVMQCQREQGDTRKDDVVKEEIFWKHIAPHFDVKLAIDDRTQVVEMWRRIGVECWQVASGDF

SEQ ID NO: 98 - DNA, 906 bp, Artificial Sequence
  T4 PNK human codon optimized nucleic acid sequence
atgaagaaaattatacttacaatcggatgccctggtagtggtaagagcacttgggcgagggaatttattgcgaagaaccctggattttataatatcaatcgagacgactaccggcagtctattatggcccacgaggaacgagacgaatacaagtataccaagaagaaagaagggattgtcacgggtatgcaatttgacaccgccaaatcaatactgtacggaggtgattcagtcaaaggcgttatcatatcagacactaacctcaatcctgaacgccgattggcatgggaaacatttgcgaaggaatacggttggaaggttgaacacaaggtgttcgatgtcccgtggaccgaactggtaaaacgcaattctaaacgaggcactaaagctgtgcccattgacgtacttcgaagtatgtacaagtccatgagagagtacctggggcttcccgtctataacggtacgccgggcaaaccgaaggcggtgatctttgacgtagatgggactctggcgaagatgaatggtcgcggaccatacgatttggaaaaatgtgacacagatgtaatcaacccaatggtagtagagcttagcaagatgtacgcattgatgggctaccaaattgtcgtggtgtccgggcgggagtcaggcacaaaagaagatccgacgaagtattatcgcatgacacggaaatgggtcgaagatatagccggggtgcctctcgttatgcaatgtcaacgagaacagggcgacacacggaaggatgacgtagtgaaggaggaaattttctggaagcatatagcgccacactttgacgttaagctcgccatcgacgaccgaactcaggtggtcgagatgtggcgacgaattggcgtagagtgttggcaagttgcatctggagatttttag

SEQ ID NO: 99 - protein, 176 aa, Artificial Sequence
  E. Coli thpR protein sequence
MSEPQRLFFAIDLPAEIREQIIHWRATHFPPEAGRPVAADNLHLTLAFLGEVSAEKEKALSLLAGRIRQPGFTLTLDDAGQWLRSRVVWLGMRQPPRGLIQLANMLRSQAARSGCFQSNRPFHPHITLLRDASEAVTIPPPGFNWSYAVTEFTLYASSFARGRTRYTPLKRWALTQ

SEQ ID NO: 100 - DNA, 531 bp, Artificial Sequence
  E. Coli thpR human codon optimized nucleic acid sequence
atgagtgagcctcaacgattgttctttgccatagatttgcctgctgaaattagagagcaaattatccattggagagccacccatttccccccagaagctggacgaccagtcgcagcggacaacctccaccttacactggcgttcttgggtgaagtgagcgccgagaaagagaaagctctctcacttctggctgggaggattcggcagccgggctttacccttactctggatgatgccggccagtggctgaggtccagggttgtctggctcggaatgaggcaaccacctagggggctcatccagctcgccaatatgctgagatcccaggccgcaaggtctggctgcttccaatcaaacaggccattccacccgcatattaccttgctcagagatgcctccgaggcagtaactattccacctcccggctttaactggagttacgccgtcacagaatttactctgtacgcctccagcttcgcccgagggagaaccaggtacacgcctttgaagcggtgggccttgacccagtag

SEQ ID NO: 101 - protein, 521 aa, Artificial Sequence
  Human PNKP protein sequence
MGEVEAPGRLWLESPPGGAPPIFLPSDGQALVLGRGPLTQVTDRKCSRTQVELVADPETRTVAVKQLGVNPSTTGTQELKPGLEGSLGVGDTLYLVNGLHPLTLRWEETRTPESQPDTPPGTPLVSQDEKRDAELPKKRMRKSNPGWENLEKLLVFTAAGVKPQGKVAGFDLDGTLITTRSGKVFPTGPSDWRILYPEIPRKLRELEAEGYKLVIFTNQMSIGRGKLPAEEFKAKVEAVVEKLGVPFQVLVATHAGLYRKPVTGMWDHLQEQANDGTPISIGDSIFVGDAAGRPANWAPGRKKKDFSCADRLFALNLGLPFATPEEFFLKWPAAGFELPAFDPRTVSRSGPLCLPESRALLSASPEVVVAVGFPGAGKSTFLKKHLVSAGYVHVNRDTLGSWQRCVTTCETALKQGKRVAIDNTNPDAASRARYVQCARAAGVPCRCFLFTATLEQARHNNRFREMTDSSHIPVSDMVMYGYRKQFEAPTLAEGFSAILEIPFRLWVEPRLGRLYCQFSEG

SEQ ID NO: 102 - DNA, 1566 bp, Artificial Sequence
  Human PNKP human codon optimized nucleic acid sequence
atgggcgaggtggaggccccgggccgcttgtggctcgagagcccccctgggggagcgccccccatcttcctgccctcggacgggcaagccctggtcctgggcaggggacccctgacccaggttacggaccggaagtgctccagaactcaagtggagctggtcgcagatcctgagacccggacagtggcagtgaaacagctgggagttaacccctcaactaccgggacccaggagttgaagccggggttggagggctctctgggggtgggggacacactgtatttggtcaatggcctccacccactgaccctgcgctgggaagagacccgcacaccagaatcccagccagatactccgcctggcacccctctggtgtcccaagatgagaagagagatgctgagctgccgaagaagcgtatgcggaagtcaaaccccggctgggagaacttggagaagttgctagtgttcaccgcagctggggtgaaaccccagggcaaggtggctggctttgatctggacgggacgctcatcaccacacgctctgggaaggtctttcccactggccccagtgactggaggatcttgtacccagagattccccgtaagctccgagagctggaagccgagggctacaagctggtgatcttcaccaaccagatgagcatcgggcgcgggaagctgccagccgaggagttcaaggccaaggtggaggctgtggtggagaagctgggggtccccttccaggtgctggtggccacgcacgcaggcttgtaccggaagccggtgacgggcatgtgggaccatctgcaggagcaggccaacgacggcacgcccatatccatcggggacagcatctttgtgggagacgcagccggacgcccggccaactgggccccggggcggaagaagaaagacttctcctgcgccgatcgcctgtttgccctcaaccttggcctgcccttcgccacgcctgaggagttctttctcaagtggccagcagccggcttcgagctcccagcctttgatccgaggactgtctcccgctcagggcctctctgcctccccgagtccagggccctcctgagcgccagcccggaggtggttgtcgcagtgggattccctggggccgggaagtccacctttctcaagaagcacctcgtgtcggccggatatgtccacgtgaacagggacacgctaggctcctggcagcgctgtgtgaccacgtgtgagacagccctgaagcaagggaaacgggtcgccatcgacaacacaaacccagacgccgcgagccgcgccaggtacgtccagtgtgcccgagccgcgggcgtcccctgccgctgcttcctcttcaccgccactctggagcaggcgcgccacaacaaccggtttcgagagatgacggactcctctcatatccccgtgtcagacatggtcatgtatggctacaggaagcagttcgaggccccaacgctggctgaaggcttctctgccatcctggagatcccgttccggctatgggtggagccgaggctggggcggctgtactgccagttctccgagggctag

SEQ ID NO: 103 - RNA, 857 nt, Artificial Sequence
  NtGFP-HDV-HH-CtGFP syntethic intron
auggugagcaagggcgaggagcuguucaccgggguggugcccauccuggucgagcuggacggcgacguaaacggccacaaguucagcguguccggcgagggcgagggcgaugccaccuacggcaagcugacccugaaguucaucugcaccaccggcaagcugcccgugcccuggcccacccucgugaccacccugaccuacggcgugcagugcuucagccgcuaccccgaccacaugaagcagcacgacuucuucaaguccgccaugcccgaaggcuacguccaggagcgcaccaucuucuuggccggcauggucccagccuccucgcuggcgccggcugggcaacaugcuucggcauggcgaaugggaccccgggacauaacuaguuaaaccaaauccuugcugaugaguccgugaggacgaaacgaguaagcucguccaaggacgacggcaacuacaagacccgcgccgaggugaaguucgagggcgacacccuggugaaccgcaucgagcugaagggcaucgacuucaaggaggacggcaacauccuggggcacaagcuggaguacaacuacaacagccacaacgucuauaucauggccgacaagcagaagaacggcaucaaggugaacuucaagauccgccacaacaucgaggacggcagcgugcagcucgccgaccacuaccagcagaacacccccaucggcgacggccccgugcugcugcccgacaaccacuaccugagcacccaguccgcccugagcaaagaccccaacgagaagcgcgaucacaugguccugcuggaguucgugaccgccgccgggaucacucucggcauggacgagcuguacaaguag

SEQ ID NO: 104 - RNA, 248 nt, Artificial Sequence
  sGCN4 5′ UTR uORFs
uuaaagaucauugaaaaauggcuugcuaaaccgauuauauuuuguuuuuaaaguagauuauuauuagaaaauuauuaagagaauuauguguuaaauuuauugaaagagaaaauuuauuuucccuuauuaauuaaaguccuuuacuuuuuuugaaaacugucaguuuuuugaagaguuauuuguuuuguuaccaauugcuaucauguacccguagaauuuuauucaagauguuuccguaacgguuaccu

SEQ ID NO: 105 - RNA, 56 nt, Artificial Sequence
  Hammerhead (HH) for 3′
  misc_feature (1)..(4): n is a, c, g, or u
  misc_feature (53)..(56): n is a, c, g, or u
nnnndwhaccggauguguuuuccggucugaugaguccgguagcggacgaawhnnnn

SEQ ID NO: 106 - RNA, 54 nt, Artificial Sequence
  Twister WT with 5 nt P1 stem
  misc_feature (1)..(5): n is a, c, g, or u
  misc_feature (50)..(54): n is a, c, g, or u
nnnnnuaacacugccaaugccggucccaagcccggauaaaaguggagggnnnnn

SEQ ID NO: 107 - RNA, 54 nt, Artificial Sequence
  Twister Mutant with 5 nt P1 stem
  misc_feature (1)..(5): n is a, c, g, or u
  misc_feature (50)..(54): n is a, c, g, or u
nnnnnuaacucuuccaaugccggucccaagcccggauaaaaguggagggnnnnn

SEQ ID NO: 108 - RNA, 54 nt, Artificial Sequence
  Twister with 5 nt P1 stem with U1A mutation
  misc_feature (1)..(5): n is a, c, g, or u
  misc_feature (50)..(54): n is a, c, g, or u
nnnnnaaacacugccaaugccggucccaagcccggauaaaaguggagggnnnnn

SEQ ID NO: 109 - RNA, 54 nt, Artificial Sequence
  Twister with 5 nt P1 stem with U1C mutation
  misc_feature (1)..(5): n is a, c, g, or u
  misc_feature (50)..(54): n is a, c, g, or u
nnnnncaacacugccaaugccggucccaagcccggauaaaaguggagggnnnnn

SEQ ID NO: 110 - RNA, 54 nt, Artificial Sequence
  Twister with 5 nt P1 stem with U1G mutation
  misc_feature (1)..(5): n is a, c, g, or u
  misc_feature (50)..(54): n is a, c, g, or u
nnnnngaacacugccaaugccggucccaagcccggauaaaaguggagggnnnnn

SEQ ID NO: 111 - RNA, 41 nt, Artificial Sequence
  Hammerhead 4 nt overhang for 5′
  misc_feature (1)..(4): n is a, c, g, or u
nnnncugaugaguccgugaggacgaaacgaguaagcucguc

SEQ ID NO: 112 - RNA, 43 nt, Artificial Sequence
  Hammerhead 6 nt overhang for 5′
  misc_feature (1)..(6): n is a, c, g, or u
nnnnnncugaugaguccgugaggacgaaacgaguaagcucguc

SEQ ID NO: 113 - RNA, 45 nt, Artificial Sequence
  Hammerhead 8 nt overhang for 5′
  misc_feature (1)..(8): n is a, c, g, or u
nnnnnnnncugaugaguccgugaggacgaaacgaguaagcucguc

SEQ ID NO: 114 - RNA, 47 nt, Artificial Sequence
  Hammerhead 10 nt overhang for 5′
  misc_feature (1)..(10): n is a, c, g, or u
nnnnnnnnnncugaugaguccgugaggacgaaacgaguaagcucguc

SEQ ID NO: 115 - RNA, 49 nt, Artificial Sequence
  Hammerhead 12 nt overhang for 5′
  misc_feature (1)..(12): n is a, c, g, or u
nnnnnnnnnnnncugaugaguccgugaggacgaaacgaguaagcucguc

SEQ ID NO: 116 - RNA, 51 nt, Artificial Sequence
  Hammerhead 14 nt overhang for 5′
  misc_feature (1)..(14): n is a, c, g, or u
nnnnnnnnnnnnnncugaugaguccgugaggacgaaacgaguaagcucguc

SEQ ID NO: 117 - RNA, 53 nt, Artificial Sequence
  Hammerhead 16 nt overhang for 5′
  misc_feature (1)..(16): n is a, c, g, or u
nnnnnnnnnnnnnnnncugaugaguccgugaggacgaaacgaguaagcucguc

SEQ ID NO: 118 - RNA, 45 nt, Artificial Sequence
  TX2 Hammerhead 4 nt overhang for 5′
  misc_feature (1)..(4): n is a, c, g, or u
nnnncugaugaguccgguagcggacgaaacgcgcuucggugcguc

SEQ ID NO: 119 - RNA, 47 nt, Artificial Sequence
  TX2 Hammerhead 6 nt overhang for 5′
  misc_feature (1)..(6): n is a, c, g, or u
nnnnnncugaugaguccgguagcggacgaaacgcgcuucggugcguc

SEQ ID NO: 120 - RNA, 49 nt, Artificial Sequence
  TX2 Hammerhead 8 nt overhang for 5′
  misc_feature (1)..(8): n is a, c, g, or u
nnnnnnnncugaugaguccgguagcggacgaaacgcgcuucggugcguc

SEQ ID NO: 121 - RNA, 51 nt, Artificial Sequence
  TX2 Hammerhead 10 nt overhang for 5′
  misc_feature (1)..(10): n is a, c, g, or u
nnnnnnnnnncugaugaguccgguagcggacgaaacgcgcuucggugcguc

SEQ ID NO: 122 - RNA, 53 nt, Artificial Sequence
  TX2 Hammerhead 12 nt overhang for 5′
  misc_feature (1)..(12): n is a, c, g, or u
nnnnnnnnnnnncugaugaguccgguagcggacgaaacgcgcuucggugcguc

SEQ ID NO: 123 - RNA, 55 nt, Artificial Sequence
  TX2 Hammerhead 14 nt overhang for 5′
  misc_feature (1)..(14): n is a, c, g, or u
nnnnnnnnnnnnnncugaugaguccgguagcggacgaaacgcgcuucggugcguc

SEQ ID NO: 124 - RNA, 57 nt, Artificial Sequence
  TX2 Hammerhead 16 nt overhang for 5′
  misc_feature (1)..(16): n is a, c, g, or u
nnnnnnnnnnnnnnnncugaugaguccgguagcggacgaaacgcgcuucggugcguc

SEQ ID NO: 125 - RNA, 55 nt, Artificial Sequence
  RzB Hammerhead for 5′
  misc_feature (1)..(6): n is a, c, g, or u
  misc_feature (10)..(14): n is a, c, g, or u
nnnnnnuaannnnncugaugagucgcugggaugcgacgaaacgccuucgggcguc

SEQ ID NO: 126 - RNA, 832 nt, Artificial Sequence
  NtGFP-HDV-CARGO-HH-CtGFP
  misc_feature (371)..(371): n is a, c, g, or u
auggugagcaagggcgaggagcuguucaccgggguggugcccauccuggucgagcuggacggcgacguaaacggccacaaguucagcguguccggcgagggcgagggcgaugccaccuacggcaagcugacccugaaguucaucugcaccaccggcaagcugcccgugcccuggcccacccucgugaccacccugaccuacggcgugcagugcuucagccgcuaccccgaccacaugaagcagcacgacuucuucaaguccgccaugcccgaaggcuacguccaggagcgcaccaucuucuuggccggcauggucccagccuccucgcuggcgccggcugggcaacaugcuucggcauggcgaaugggacnuccuugcugaugaguccgugaggacgaaacgaguaagcucguccaaggacgacggcaacuacaagacccgcgccgaggugaaguucgagggcgacacccuggugaaccgcaucgagcugaagggcaucgacuucaaggaggacggcaacauccuggggcacaagcuggaguacaacuacaacagccacaacgucuauaucauggccgacaagcagaagaacggcaucaaggugaacuucaagauccgccacaacaucgaggacggcagcgugcagcucgccgaccacuaccagcagaacacccccaucggcgacggccccgugcugcugcccgacaaccacuaccugagcacccaguccgcccugagcaaagaccccaacgagaagcgcgaucacaugguccugcuggaguucgugaccgccgccgggaucacucucggcauggacgagcuguacaaguag

SEQ ID NO: 127 - RNA, 370 nt, Artificial Sequence
  NtGFP-HDV
auggugagcaagggcgaggagcuguucaccgggguggugcccauccuggucgagcuggacggcgacguaaacggccacaaguucagcguguccggcgagggcgagggcgaugccaccuacggcaagcugacccugaaguucaucugcaccaccggcaagcugcccgugcccuggcccacccucgugaccacccugaccuacggcgugcagugcuucagccgcuaccccgaccacaugaagcagcacgacuucuucaaguccgccaugcccgaaggcuacguccaggagcgcaccaucuucuuggccggcauggucccagccuccucgcuggcgccggcugggcaacaugcuucggcauggcgaaugggac

SEQ ID NO: 128 - RNA, 461 nt, Artificial Sequence
  HH-CtGFP
uccuugcugaugaguccgugaggacgaaacgaguaagcucguccaaggacgacggcaacuacaagacccgcgccgaggugaaguucgagggcgacacccuggugaaccgcaucgagcugaagggcaucgacuucaaggaggacggcaacauccuggggcacaagcuggaguacaacuacaacagccacaacgucuauaucauggccgacaagcagaagaacggcaucaaggugaacuucaagauccgccacaacaucgaggacggcagcgugcagcucgccgaccacuaccagcagaacacccccaucggcgacggccccgugcugcugcccgacaaccacuaccugagcacccaguccgcccugagcaaagaccccaacgagaagcgcgaucacaugguccugcuggaguucgugaccgccgccgggaucacucucggcauggacgagcuguacaaguag

SEQ ID NO: 129 - RNA, 3724 nt, Artificial Sequence
  Nt-miniDys (deltaH2-R15)
augcuuuggugggaagaaguagaggacuguuaugaaagagaagauguucaaaagaaaacauucacaaaauggguaaaugcacaauuuucuaaguuugggaagcagcauauugagaaccucuucagugaccuacaggaugggaggcgccuccuagaccuccucgaaggccugacagggcaaaaacugccaaaagaaaaaggauccacaagaguucaugcccugaacaaugucaacaaggcacugcggguuuugcagaacaauaauguugauuuagugaauauuggaaguacugacaucguagauggaaaucauaaacugacucuugguuugauuuggaauauaauccuccacuggcaggucaaaaauguaaugaaaaauaucauggcuggauugcaacaaaccaacagugaaaagauucuccugagcuggguccgacaaucaacucguaauuauccacagguuaauguaaucaacuucaccaccagcuggucugauggccuggcuuugaaugcucucauccauagucauaggccagaccuauuugacuggaauagugugguuugccagcagucagccacacaacgacuggaacaugcauucaacaucgccagauaucaauuaggcauagagaaacuacucgauccugaagauguugauaccaccuauccagauaagaaguccaucuuaauguacaucacaucacucuuccaaguuuugccucaacaagugagcauugaagccauccaggaaguggaaauguugccaaggccaccuaaagugacuaaagaagaacauuuucaguuacaucaucaaaugcacuauucucaacagaucacggucagucuagcacagggauaugagagaacuucuuccccuaagccucgauucaagagcuaugccuacacacaggcugcuuaugucaccaccucugacccuacacggagcccauuuccuucacagcauuuggaagcuccugaagacaagucauuuggcaguucauugauggagagugaaguaaaccuggaccguuaucaaacagcuuuagaagaaguauuaucguggcuucuuucugcugaggacacauugcaagcacaaggagagauuucuaaugauguggaaguggugaaagaccaguuucauacucaugagggguacaugauggauuugacagcccaucagggccggguugguaauauucuacaauugggaaguaagcugauuggaacaggaaaauuaucagaagaugaagaaacugaaguacaagagcagaugaaucuccuaaauucaagaugggaaugccucaggguagcuagcauggaaaaacaaagcaauuuacauagaguuuuaauggaucuccagaaucagaaacugaaagaguugaaugacuggcuaacaaaaacagaagaaagaacaaggaaaauggaggaagagccucuuggaccugaucuugaagaccuaaaacgccaaguacaacaacauaaggugcuucaagaagaucuagaacaagaacaagucagggucaauucucucacucacaugguggugguaguugaugaaucuaguggagaucacgcaacugcugcuuuggaagaacaacuuaagguauugggagaucgaugggcaaacaucuguagauggacagaagaccgcuggguucuuuuacaagacauccuucucaaauggcaacgucuuacugaagaacagugccuuuuuagugcauggcuuucagaaaaagaagaugcagugaacaagauucacacaacuggcuuuaaagaucaaaaugaaauguuaucaagucuucaaaaacuggccguuuuaaaagcggaucuagaaaagaaaaagcaauccaugggcaaacuguauucacucaaacaagaucuucuuucaacacugaagaauaagucagugacccagaagacggaagcauggcuggauaacuuugcccgguguugggauaauuuaguccaaaaacuugaaaagaguacagcacagauuucacaggaaauuucuuaugugccuucuacuuauuugacugaaaucacucaugucucacaagcccuauuagaaguggaacaacuucucaaugcuccugaccucugugcuaaggacuuugaagaccucuuuaagcaagaggagucucugaagaauauaaaagauagucuacaacaaagcucaggucggauugacauuauucauagcaagaagacagcagcauugcaaagugcaacgccuguggaaagggugaagcuacaggaagcucucucccagcuugauuuccaaugggaaaaaguuaacaaaauguacaaggaccgacaagggcgauuugacagauccguugagaaauggcggcguuuucauuaugauauaaagauauuuaaucaguggcuaacagaagcugaacaguuucucagaaagacacaaauuccugagaauugggaacaugcuaaauacaaaugguaucuuaaggaacuccaggauggcauugggcagcggcaaacuguugucagaacauugaaugcaacuggggaagaaauaauucagcaauccucaaaaacagaugccaguauucuacaggaaaaauugggaagccugaaucugcgguggcaggaggucugcaaacagcugucagacagaaaaaagaggcuagaagaacaaaagaauaucuugucagaauuucaaagagauuuaaaugaauuuguuuuaugguuggaggaagcagauaacauugcuaguaucccacuugaaccuggaaaagagcagcaacuaaaagaaaagcuugagcaagucaaguuacugguggaagaguugccccugcgccagggaauccucaaacaauuaaaugaaacuggaggacccgugcuuguaagugcucccauaagcccagaagagcaagauaaacuugaaaauaagcucaagcagacaaaucuccaguggauaaagguuuccagagcuuuaccugagaaacaaggagaaauugaagcucaaauaaaagaccuugggcagcuugaaaaaaagcuugaagaccuugaagagcaguuaaaucaucugcugcugugguuaucuccuauuaggaaucaguuggaaauuuauaaccaaccaaaccaagaaggaccauuugacguuaaggaaacugaaauagcaguucaagcuaaacaaccggauguggaagagauuuugucuaaagggcagcauuuguacaaggaaaaaccagccacucagccagugaagaggaaguuagaagaccuguccucugaguggaaggcgguaaaccguuuacuucaagagcugagggcaaagcagccugaccuagcuccuggacugaccacuauuggagccucuccuacucagacuguuacucuggugacacaaccugugguuacuaaggaaacugccaucuccaaacuagaaaugccaucuuccuugauguuggagguaccugcucuggcagauuucaaccgggcuuggacagaacuuaccgacuggcuuucucugcuugaucaaguuauaaaaucacaacgcgugauggugggcgaccuugaggauaucaacgagaugaucaucaagcagaaggcaacaaugcaggauuuggaacagaggcguccccaguuggaagaacucauuaccgcugcccaaaauuugaaaaacaagaccagcaaucaagaggcuagaacaaucauuacggaucgaauugaaagaauucagaaucagugggaugaaguacaag

SEQ ID NO: 130 - RNA, 3362 nt, Artificial Sequence
  Ct-miniDys (deltaH2-R15)
aacaccuucagaaccggaggcaacaguugaaugaaauguuaaaggauucaacacaauggcuggaagcuaaggaagaagcugagcaggucuuaggacaggccagagccaagcuggagucauggaaggagggucccuauacaguagaugcaauccaaaagaaaaucacagaaaccaagcaguuggccaaagaccuccgccaguggcagacaaauguagauguggcaaaugacuuggcccugaaacuucuccgggauuauucugcagaugauaccagaaaaguccacaugauaacagagaauaucaaugccucuuggagaagcauucauaaaagggugagugagcgagaggcugcuuuggaagaaacucauagauuacugcaacaguucccccuggaccuggaaaaguuucuugccuggcuuacagaagcugaaacaacugccaauguccuacaggaugcuacccguaaggaaaggcuccuagaagacuccaagggaguaaaagagcugaugaaacaauggcaagaccuccaaggugaaauugaagcucacacagauguuuaucacaaccuggaugaaaacagccaaaaaauccugagaucccuggaagguuccgaugaugcaguccuguuacaaagacguuuggauaacaugaacuucaaguggagugaacuucggaaaaagucucucaacauuaggucccauuuggaagccaguucugaccaguggaagcgucugcaccuuucucugcaggaacuucugguguggcuacagcugaaagaugaugaauuaagccggcaggcaccuauuggaggcgacuuuccagcaguucagaagcagaacgaugugcauagggccuucaagagggaauugaaaacuaaagaaccuguaaucaugaguacucuugagacuguacgaauauuucugacagagcagccuuuggaaggacuagagaaacucuaccaggagcccagagagcugccuccugaggagagagcccagaaugucacucggcuucuacgaaagcaggcugaggaggucaauacugagugggaaaaauugaaccugcacuccgcugacuggcagagaaaaauagaugagacccuugaaagacuccgggaacuucaagaggccacggaugagcuggaccucaagcugcgccaagcugaggugaucaagggauccuggcagcccgugggcgaucuccucauugacucucuccaagaucaccuggagaaagucaaggcacuucgaggagaaauugcgccucugaaagagaacgugagccacgucaaugaccuugcucgccagcuuaccacuuugggcauucagcucucaccguauaaccucagcacucuggaagaccugaacaccagauggaagcuucugcagguggccgucgaggaccgagucaggcagcugcaugaagcccacagggacuuugguccagcaucucagcacuuucuuuccacgucuguccagggucccugggagagagccaucucgccaaacaaagugcccuacuauaucaaccacgagacucaaacaacuugcugggaccaucccaaaaugacagagcucuaccagucuuuagcugaccugaauaaugucagauucucagcuuauaggacugccaugaaacuccgaagacugcagaaggcccuuugcuuggaucucuugagccugucagcugcaugugaugccuuggaccagcacaaccucaagcaaaaugaccagcccauggauauccugcagauuauuaauuguuugaccacuauuuaugaccgccuggagcaagagcacaacaauuuggucaacgucccucucugcguggauaugugucugaacuggcugcugaauguuuaugauacgggacgaacagggaggauccguguccugucuuuuaaaacuggcaucauuucccuguguaaagcacauuuggaagacaaguacagauaccuuuucaagcaaguggcaaguucaacaggauuuugugaccagcgcaggcugggccuccuucugcaugauucuauccaaauuccaagacaguugggugaaguugcauccuuugggggcaguaacauugagccaaguguccggagcugcuuccaauuugcuaauaauaagccagagaucgaagcggcccucuuccuagacuggaugagacuggaaccccaguccaugguguggcugcccguccugcacagaguggcugcugcagaaacugccaagcaucaggccaaauguaacaucugcaaagaguguccaaucauuggauucagguacaggagucuaaagcacuuuaauuaugacaucugccaaagcugcuuuuuuucuggucgaguugcaaaaggccauaaaaugcacuaucccaugguggaauauugcacuccgacuacaucaggagaagauguucgagacuuugccaagguacuaaaaaacaaauuucgaaccaaaagguauuuugcgaagcauccccgaaugggcuaccugccagugcagacugucuuagagggggacaacauggaaacucccguuacucugaucaacuucuggccaguagauucugcgccugccucguccccucagcuuucacacgaugauacucauucacgcauugaacauuaugcuagcaggcuagcagaaauggaaaacagcaauggaucuuaucuaaaugauagcaucucuccuaaugagagcauagaugaugaacauuuguuaauccagcauuacugccaaaguuugaaccaggacuccccccugagccagccucguaguccugcccagaucuugauuuccuuagagagugaggaaagaggggagcuagagagaauccuagcagaucuugaggaagaaaacaggaaucugcaagcagaauaugaccgucuaaagcagcagcacgaacauaaaggccuguccccacugccguccccuccugaaaugaugcccaccucuccccagaguccccgggaugcugagcucauugcugaggccaagcuacugcgucaacacaaaggccgccuggaagccaggaugcaaauccuggaagaccacaauaaacagcuggagucacaguuacacaggcuaaggcagcugcuggagcaaccccaggcagaggccaaagugaauggcacaacgguguccucuccuucuaccucucuacagagguccgacagcagucagccuaugcugcuccgagugguuggcagucaaacuucggacuccaugggugaggaagaucuucucaguccuccccaggacacaagcacaggguuagaggaggugauggagcaacucaacaacuccuucccuaguucaagaggaagaaauaccccuggaaagccaaugagagaggacacaauguaa